(19) United States
(12) Patent Application Publication
(10) Pub. No.: US 2025/0205280 A1
(43) Pub. Date: Jun. 26, 2025

(54) Methods and Compositions Comprising V beta 17 Bispecific T Cell Engagers and Bioengineered Virus Specific Lymphocytes

(71) Applicant: Janssen Biotech, Inc., Horsham, PA (US)

(72) Inventors: Rajkumar Ganesan, Raritan, NJ (US); Iqbal s. Grewal, Raritan, NJ (US); Michael Riis Hansen, Raritan, NJ (US)

(21) Appl. No.: 18/837,262

(22) PCT Filed: Feb. 7, 2023
(86) PCT No.: PCT/IB2023/051080

§ 371 Date: Aug. 9, 2024

Related U.S. Application Data
(60) Provisional application No. 63/308,423, filed on Feb. 9, 2022.
(60) Provisional application No. 63/308,425, filed on Feb. 9, 2022.
(60) Provisional application No. 63/308,426, filed on Feb. 9, 2022.
(60) Provisional application No. 63/308,427, filed on Feb. 9, 2022.
(60) Provisional application No. 63/308,428, filed on Feb. 9, 2022.

(51) Int. Cl. A61K 35/17; A61K 39/00; A61K 40/11; A61K 40/42; A61K 40/46; A61P 35/00; C07K 16/28; C07K 16/30; C12N 5/00; C12N 5/0783

(52) U.S. Cl. CPC A61K 35/17; A61K 40/11; A61K 40/421; A61K 40/46; A61P 35/00; C07K 16/2809; C07K 16/2827; C07K 16/2866; C07K 16/3069; C12N 5/0018; C12N 5/0636; A61K 2039/505; C07K 2317/31; C07K 2317/73; C12N 2501/2302; C12N 2501/998

(57) ABSTRACT

Provided is a pharmaceutical composition or combination comprising: (i) an isolated population of cells comprising Vβ17+CD8+ T cells, (ii) one or more T cell engagers; and (iii) a pharmaceutically acceptable excipient. Also provided are methods of using the isolated population of cells comprising Vβ17+CD8+ T cells and the T cell engagers for redirecting a T cell to a target cell, inhibiting the growth or proliferation of a target cell, eliminating a target cell, or treating a disease or disorder.

### CROSS REFERENCE TO RELATED APPLICATION

This application claims the benefit of U.S. Provisional Patent Application No. 63/308,423, filed Feb. 9, 2022, U.S. Provisional Patent Application No. 63/308,425, filed Feb. 9, 2022, U.S. Provisional Patent Application No. 63/308,426, filed Feb. 9, 2022, U.S. Provisional Patent Application No. 63/308,427, filed Feb. 9, 2022, U.S. Provisional Patent Application No. 63/308,428, filed Feb. 9, 2022, the disclosures of which are incorporated by reference herein in their entireties.

### REFERENCE TO SEQUENCE LISTING SUBMITTED ELECTRONICALLY

This application contains a sequence listing, which is submitted electronically. The content of the electronic sequence listing (065768-137WO1_Sequence Listing.xml; size: 108,233 bytes; and date of creation: Jan. 13, 2023) is herein incorporated by reference in its entirety.

### 1. FIELD

Provided herein, in some embodiments, are bioengineered lymphocytes, including influenza specific Vβ17⁺CD8⁺ T cells, for use in combination with multispecific T cell engagers for treating a disease or disorder.

### 2. BACKGROUND

Considerable progress has been made in the development of immunotherapeutics that are designed to harness the power of tumor-killing capacity of T cells (see, e.g., Ribas, A. & Wolchok, J. D., Science, 359(6382): 1350-1355 (2018)). One of the key strategies to realize the full potential of T cells is to mobilize T cells using bi-specific T cell engagers, which enable efficient activation of polyclonal T cells and recruitment of pan T cells through binding to CD3 expressed on T cells and a tumor specific antigen expressed on tumor cells (see Velasquez, M. P. et al, Blood, 131(1): 30-38 (2018)).

Although redirecting the cytotoxicity of T cells by CD3 bispecific T cell engagers has resulted in remarkable clinical activity, this approach is often accompanied by immune-related adverse events (IRAEs). Prime reason for IRAEs is the pan activation of T cells via rapid CD3 signaling, which leads to severe cytokine storm. IRAEs limit the dose of the drug and result in a narrow therapeutic index. In addition, there is limited efficacy of the current generation of CD3 bispecific T cell engagers in solid tumors, which may be due to inefficient recruitment of T cells or lack of penetration of recruited T cells to epithelial tissues. Therefore, there is a need for improved redirection technology for T cell therapies.

### 3. SUMMARY

In one aspect, provided herein is a pharmaceutical composition comprising: (i) an isolated population of cells comprising Vβ17+CD8+ T cells, and (ii) one or more T cell engagers. In some embodiments, the composition provided herein further comprises a pharmaceutically acceptable excipient.

In some embodiments, the isolated population of cells comprising Vβ17+CD8+ T cells has been activated or enriched via contacting a M1 peptide derived from human influenza A virus (M158-66) with a population of cells comprising T cells. In some embodiments, the M1 peptide comprises the amino acid sequence of GILGFVFTL (SEQ ID NO:1).

In some embodiments, the isolated population of cells comprising Vβ17+CD8+ T cells has been activated or enriched via further contacting IL-2 with the population of cells comprising T cells.

In some embodiments, the population of cells comprising T cells has been cultured ex vivo in a medium comprising the M1 peptide and/or IL-2.

In some embodiments, the ex vivo culturing comprises: (i) culturing the population of cells comprising T cells ex vivo in a medium comprising the M1 peptide and IL-2; (ii) culturing the population of cells comprising T cells ex vivo in a medium comprising the M1 peptide, and then culturing the population of cells comprising T cells ex vivo in a medium comprising IL-2; or (iii) culturing the population of cells comprising T cells ex vivo in a medium comprising the M1 peptide, then culturing the population of cells comprising T cells ex vivo in a medium comprising the M1 peptide and IL-2; and then culturing the population of cells comprising T cells ex vivo in a medium comprising IL-2.

In some embodiments, the population of cells comprising T cells has been cultured ex vivo for at least 1, 2, 3, 4, 5, 6, 7, 8, 9, 10, 11, 12, 13, or 14 days.

In some embodiments, the population of cells comprising T cells is a population of whole peripheral blood mononuclear cells (PBMCs). In some embodiments, the population of the whole PBMCs is from a healthy donor. In some embodiments, the population of the whole PBMCs is from an unhealthy donor.

In some embodiments, the Vβ17+CD8+ T cells have been isolated from the population of cells comprising T cells after contacting the population of cells comprising T cells with the M1 peptide and/or IL-2.

In some embodiments, the percent of Vβ17+CD8+ T cells in the isolated population of cells is more than 6%, 10%, 15%, 20%, 25%, 30%, 35%, 40%, 45%, 50%, 55%, 60%, 65%, 70%, 75%, 80%, 85%, or 90%.

In some embodiments, at least part of the Vβ17+CD8+ T cells express a cell surface receptor capable of binding to the M1 peptide.

In some embodiments, each of the T cell engagers is a multi-specific antibody. In some embodiments, each of the T cell engagers comprises: (1) a first binding domain that binds to an antigen expressed on a Vβ17+CD8+ T cell; and (2) a second binding domain that binds to an antigen expressed on an unhealthy cell.

In some embodiments, the antigen expressed on the Vβ17+CD8+ T cell is T Cell Receptor Beta Variable 19 (TRBV19) or Vβ17.

In some embodiments, the unhealthy cell is a cancer cell. In some embodiments, the cancer cell is a blood cancer cell or a solid tumor cancer cell. In some embodiments, the antigen expressed on the unhealthy cell is a tumor-associated antigen (TAA). In some embodiments, the TAA is CD123, ILIRAP, PSMA, or B7H3.

In some embodiments, each of the T cell engagers is a TRBV19xTAA or Vβ17xTAA bi-specific antibody.

In another aspect, provided herein is a method for redirecting a T cell to a target cell, comprising contacting the target cell with one or more T cell engagers in the presence of an isolated population of cells comprising Vβ17+CD8+ T cells, wherein the contacting directs the Vβ17+CD8+ T cell to the target cell.

In another aspect, provided herein is a method for inhibiting the growth or proliferation of a target cell, comprising contacting the target cell with one or more T cell engagers in the presence of an isolated population of cells comprising Vβ17+CD8+ T cells, wherein the contacting results in the inhibition of the growth or proliferation of the target cell.

In yet another aspect, provided herein is a method for eliminating a target cell, comprising contacting the target cell with one or more T cell engagers in the presence of an isolated population of cells comprising Vβ17+CD8+ T cells, wherein the contacting results in the elimination of the target cell.

In yet another aspect, provided herein is a method for treating a disease or disorder in a subject comprising administering to the subject: (i) a therapeutically effective amount of an isolated population of cells comprising Vβ17+CD8+ T cells, and (ii) a therapeutically effective amount of one or more T cell engagers. In some embodiments, the disease or disorder is cancer. In some embodiments, the cancer is a blood cancer. In some embodiments, the cancer is a solid tumor cancer. In some embodiments, the subject is a human subject in need thereof.

### 4. BRIEF DESCRIPTION OF THE FIGURES

FIG. 1: illustrates the mechanism of action for redirecting the Vβ17⁺αβT cells by a bispecific antibody for specific killing of tumor cells.

FIG. 2A illustrates a molecular model (using crystal structure PBD: 5HHM) of TCR Vβ17 and residues in the epitope are highlighted in the sphere representation. FIG. 2B shows binding kinetics of mouse anti-human anti-TCR-Vβ17 mAb to recombinant Vβ17-Fc antigen by SPR at 25° C. Different concentrations of antigen (100 nM, from top to bottom in the plot) were flowed through anti-Vβ17 mAb that was captured on the surface. Experimental data (black line) and 1:1 Langmuir binding fitting (red line) is shown. The association phase between (first ˜250 sec) is follow by the dissociation phase. Global fitting to a 1:1 simple Langmuir binding model resulted a K_{D}=5.05 nM. FIG. 2C shows humanization of murine anti-Vβ17 antibody. Humanization of murine clone B17B1 was performed following the process outlined by Singh et al. Based on sequence homology, IGHV4-59*01 and IGKV1-110*01 were chosen for framework adaption. FIG. 2D shows the epitope and paratope mapping for the mouse anti-human anti-TCR-Vβ17 mAb and Vβ17 fused to human Fc. The top panel shows that the peptide region, ⁵⁶SQIVNDFQKGDIAEG⁷⁵, was protected by mAb B17B1; the middle panel shows a molecular model of the Fab with residues in the paratope highlighted; the lower panel shows HDX paratope mapping on the anti-Vβ17 mAb.

FIGS. 3A-3D show the frequency of Vβ17+ T cells among the CD3 pan T cell population. FIG. 3A shows the frequency of Vβ17⁺ cells among CD8⁺ T cells (upper) and the distribution of Vβ17⁺ T cells among CD8⁺ T cell from healthy donors (HD), Lung cancer patients, AML patients and NHL patients (lower). FIG. 3B summarizes the frequency of Vβ17⁺ cells among CD8⁺ T cells from HLA-A2⁻ and HLA-A2⁺ healthy individuals. FIG. 3C shows the frequency of Vβ17⁺ cells among CD4⁺ T cells (upper) and the distribution of Vβ17⁺ T cells among CD4⁺ T cell from HLA-A2⁺ and HLA-A2⁻ donors (lower). FIG. 3D shows the distribution of Vβ17⁺ and Vβ17⁻ cells among CD4 T cell from healthy donors (HD), Lung cancer patients, AML patients and NHL patients. Each symbol represent data from a healthy donor or cancer patient.

FIGS. 4A-4B show that Vβ17⁺CD8⁺ T cells can be expanded ex-vivo. FIG. 4A shows the frequency of Vβ17⁺ cells among CD8⁺ and CD4⁺ T cells on day 0 (top row) and day 14 (middle row) of M1 peptide+IL-2 stimulation and IL-2 stimulation alone (bottom row). Numbers below gates represent the frequency of gated population among CD8⁺ and CD4⁺ T cells. FIG. 4B shows the frequency of Vβ17⁺ cells among CD8⁺ T cells of whole PBMCs from healthy individuals on day 0 and 14 of the M1 peptide stimulation. Each dot represents the data from a healthy donor. Statistical significance (p values) was calculated with two-tailed paired t-test (* indicates p<0.05, **p<0.01, ***p<0.001, ****p<0.0001 and ns suggests p>0.05). FIG. 4C shows Vβ17⁺CD8⁺ T cells, not Vβ17⁺CD4⁺ T cells, can be activated using Influenza M1 peptide: Numbers in histogram overlays refer to the frequency of Vβ17⁺CD8⁺ and Vβ17⁺CD4⁺ cells that were positive for CD25, CD71, CD69, CD107a surface expression, Granzyme B and perforin intracellular expression. FIG. 4D summarizes the frequency of Vβ17⁺CD8⁺ cells positive for CD25, CD69, CD71, CD107a, Granzyme B, perforin stimulated with 11-2 alone or M1 peptide+IL-2 on day 0 and day 8 of PBMCs culture. Representative data of n=7 donors from 3 independent experiments are shown.

FIGS. 5A-5C show Vβ17 bispecific binding to Vβ17⁺CD8⁺ T cells and TAA expressing tumor cells. FIG. 5A show binding of bispecific antibodies to Tumor Associated Antigen (TAA) expressing target cells and enriched CD8⁺ T cells from PBMCs after being stimulated with M1 peptide for fourteen days. Green and red lines reflect the indicated Vβ17 bispecific antibody and its corresponding Vβ17 null arm bispecific control antibodies respectively. FIG. 5B (upper panels) shows the depletion efficacy of Vβ17⁺ cells among total CD8⁺ T cells, where Vβ17⁺ cells were FACS-sort depleted from enriched CD8⁺ T cells of whole PBMCs from healthy individuals. Numbers in quadrants represent the frequency of the respective population. FIG. 5B (lower panels) reflects the binding of Vβ17/CD123 bispecific antibodies and Vβ17/NULL antibodies respectively at indicated concentrations to total CD8⁺ T cells (green and red lines) and CD8⁺ T cells depleted of Vβ17⁺ cells (Magenta and blue lines). FIG. 5C (upper panels) shows the staining of anti-CD123 mAb, isotype control Ab and FMO control respectively on CD123 TAA expressing Kasumi-3 (upper left) and non-expressing K-562 (upper right) cell lines. FIG. 5C (lower panels) shows the binding of Vβ17/CD123 and Vβ17/NULL bispecific antibodies respectively to Kasumi-3 (lower left) and K-562 (lower right) cell lines at indicated concentrations.

FIGS. 6A-6B show Vβ17 bispecific mediated redirection of T cells effectively eliminate liquid and solid tumors. FIG. 6A represent the frequency of specific target cell lysis at the indicated of concentration of Vβ17 bispecific antibodies and their respective Vβ17/NULL arm controls. FIG. 6B shows the frequency (mean±SEM) of CFSE labelled target (kasumi-3) cell lysis upon co-culture with enriched Pan-T cells or Pan-T cells depleted of Vβ17⁺ cells respectively in the absence or presence of 50 ng/mL Vβ17/CD123 and CD3/CD123 bispecific antibodies respectively at indicated ET ratios. Representative data of n=3 donors from 2 independent experiments is shown.

FIGS. 7A-7C show that Vβ17/CD123 bispecific antibody potently mediates activation, differentiation, proliferation and effector functions of Vβ17⁺CD8⁺ T cells among whole PBMCs. FIG. 7A: shows the frequency of Vβ17⁻CD8⁺ and Vβ17⁺CD8⁺ T cells among CD3⁺ T cells (left panel) and CD123⁺ cells among CD3⁻ T cells (right panel). FIG. 7B show the mean (±SEM) frequency of Vβ17⁺ and Vβ17⁻ cells among CD8 T cells that are positive for their surface expression of CD25, CD71 (activation markers), intracellular Granzyme B expression (differentiation markers), CFSE dilution (proliferation profile) among whole PBMCs (effector profile) upon culture in the absence or presence of indicated bispecific antibodies. FIG. 7C shows the elimination of CD123⁺ target cells among whole PBMCs after day one (left panel) and five (right panel) of the culture in the absence or presence of Vβ17/CD123, and CD3/CD123 bispecific antibodies. Each dot represent data from a healthy donor. Representative data of n=7 donors from 3 independent experiments for FIG. 7A and FIG. 7B and n=4 donors from two independent experiments for FIG. 7C are shown. Dotted line in graphs represents the mean (±SEM) frequency value from no bispecific antibody (NBS) control well.

FIGS. 8A-8D show Vβ17/CD123 bispecific selective recruits, activates Vβ17⁺ T cells and mediates their cytotoxicity.

FIG. 9 shows that Vβ17+ T cell selective redirection do not elicit cytokine storm compared to Pan− T cell re-direction. Representative data of n=3 donors from one independent experiment is shown. Background gray bars in the left panel, corresponds to right x-axis, refer to the frequency of Vβ17⁺ T cells among total lives on indicated days of the culture period.

FIGS. 10A-10B show Vβ17⁺ T cell redirection effectively eliminates tumors in xenograft model. FIG. 10A (upper panels) shows the experiment schedule, where six to eight weeks old NSG mice were subcutaneously injected with KG-1 cells as described in detail in methods section; seven days post s.c. injection, mice were randomized based on the tumor size (˜50-70 mm³) and segregated into five groups of five mice each; after the randomization (Day 1), twenty million enriched Pan-T cells were intravenously administered to each mouse, followed by an i.v. injection of 10 μg of bispecific antibody; bispecific antibody treatment was repeated on day 3, 5, 8 & 10 of the experiment. As a control group, mice were either injected tumor alone or mice were injected with tumor plus pan-T cells without bispecific injection. FIG. 10A (lower panels) shows mean (±SEM) tumor volume of mice from all groups (n=5/group) either untreated or treated with indicated bispecifics on day 1, 3, 5, 8 and 10 at specified time points. Statistical analysis carried out by Two-way ANOVA followed by Bonferroni post tests using Graph Pad Prism. ***p<0.001 when respective test groups were compared with PBS group. FIG. 10B (upper panels) shows the experiment schedule, where established KG-1 tumor (around 500-600 mm³; 25 days post KG-1 cells injection) bearing NSG mice were intravenously injected with DIL dye labelled enriched CD8⁺Vβ17⁺ T cells (around 6 million cells/mouse), followed by an i.v. injection of 10 μg of Vβ17/CD123 bispecific antibody; post 24 and 48 hours injection of bispecific antibody, mice were euthanized and organs were ex-vivo imaged. FIG. 10B (middle panels) shows fluorescence intensity in representative images, referring to the abundance of adoptively transferred Dil dye-labelled Vβ17⁺CD8⁺ T cells in various organs of mice. FIG. 10B (lower panels) shows the mean (±SEM) signal intensity of Dil dye labeled cells in stated organs post 24 and 48 hours of injection. N=3 mice per group.

FIGS. 11A-11C show Vβ17/CD123 bispecific antibody selectively activates and mediates metabolic modulation of Vβ17⁺ cells. FIG. 11A shows the mean frequency (±SEM) of Vβ17⁺ and Vβ17⁻CD8 T cells that were positive for CD25, CD69 and CD71 (activation) surface expression in the absence or presence of Vβ17/CD123, Vβ17/Null and CD3/CD123, CD3/Null bispecific antibodies. FIG. 11B shows the mean frequency (±SEM) of Vβ17⁺ and Vβ17⁻CD8 T cells dependence on Mitochondria or Glycolysis in the presence of indicated bispecific antibodies or their respective Null arm controls. n=4 healthy donors for bispecific antibodies and 2 donors for Null arm controls from 2 independent experiments. FIG. 11C shows the mean frequency (±SEM) of Vβ17⁺ and Vβ17⁻CD4 T cells that were positive for CD25, CD69 and CD71 (activation) surface expression and CFSE dilution (proliferation) in the presence or absence of bispecific antibodies.

FIG. 12 illustrates low donor heterogeneity in Vβ17/CD123 bispecific mediated Pan-T cell cytotoxicity. FIG. 12 (upper panels) shows the percentage of specific target cell lysis (% 7-AAD⁺ cells) in the presence of Vβ17/CD123 (left) or CD3/CD123 (right) bispecific antibodies and their respective null arm controls. n=5 donors from a single experiment. FIG. 12 (lower panels) shows EC50 values for bispecific antibodies and their respective Null arm controls. ND: Not Determined.

FIGS. 13A-13C show Vβ17⁺ T cell redirection exhibit durable cytotoxicity with minimal cellular senescence/exhaustion. FIG. 13A shows the frequency (mean±SEM) of target cell killing (% 7-AAD⁺ among CFSE/CTV labelled target cells) on indicated time points (days) in the presence of Vβ17/CD123 and CD3/CD123 bispecific antibody respectively. Dotted line refers to target alone cell death (green bars). Arrows (red) mirror the target (Kasumi-3) cell re-challenge on indicated time points. Arrows (violet) point to IL-2 addition to the culture wells on indicated time points. n=4 donors from 2-3 independent experiments. FIG. 13B depicts the frequency (mean±SEM) of Vβ17⁺ cells among CD8 T cells on indicated days of co-culture period from FIG. 13A (CD8 T cells co-culture with target cells in the presence of Vβ17/CD123 bispecific antibody). FIG. 13C shows the mean (±SEM) gMFI (for PD1, Lag3) or frequency (for TIGIT and CD57) of Vβ17+CD8+ T cells or Vβ17−CD8+ cells. f. n=2 donors from one experiment for FIG. 13B and FIG. 13C.

FIGS. 14A-14B show Vβ17⁺ T cells retain effector and proliferative abilities, albeit less, under hypoxia conditions. FIG. 14A shows the frequency (mean±SEM) of specific target cell lysis (cytotoxicity) under normoxia/hypoxia at indicated ET ratios with the indicated bispecific antibodies. n=5 donors from two independent experiments. FIG. 14B shows the frequency of Vβ17⁺ cells among CD8 T cells on day 5 of co-culture under normoxia or hypoxia with indicated bispecific antibody/Control at an ET ratio of 1:1. n=3 donors for a single experiment.

FIG. 15A shows that activation and proliferation of CD8+ (Vβ17+ and Vβ17− T cells) are not regulated by suppressive cytokines like IL-10 and TGFβ. FIG. 15B shows that activation and proliferation of CD8+ (Vβ17+ and Vβ17− T cells) are not regulated by M2 macrophages.

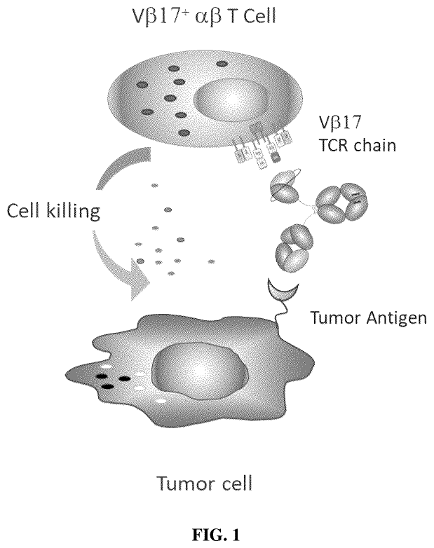

### 5. DETAILED DESCRIPTION

The present disclosure is based, in part, on novel methods or processes for redirecting bioengineered lymphocytes, including influenza specific Vβ17⁺CD8⁺ T cells, using multispecific T cell engagers. Such methods or processes can be used for making cellular therapies for treating a disease or disorder.

### 5.1. Definitions

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2d ed. 2010). Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments thereof (e.g., domain antibodies), as described below. An antibody can be human, humanized, chimeric and/or affinity matured, as well as an antibody from other species, for example, mouse, rabbit, llama, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). Antibodies also include, but are not limited to, synthetic antibodies, recombinantly produced antibodies, single domain antibodies including from Camelidae species (e.g., llama or alpaca) or their humanized variants, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Plückthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies. Antibodies may be neither agonistic nor antagonistic.

An “antigen” is a structure to which an antibody can selectively bind. A target antigen may be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell.

An “intact” antibody is one comprising an antigen-binding site as well as a CL and at least heavy chain constant regions, CH1, CH2 and CH3. The constant regions may include human constant regions or amino acid sequence variants thereof. In certain embodiments, an intact antibody has one or more effector functions.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (koff) to association rate (kon) of a binding molecule (e.g., an antibody) to a monovalent antigen (koff/kon) is the dissociation constant KD, which is inversely related to affinity. The lower the KD value, the higher the affinity of the antibody. The value of KD varies for different complexes of antibody and antigen and depends on both kon and koff. The dissociation constant KD for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

In connection with the binding molecules described herein terms such as “bind to,” “that specifically bind to,” and analogous terms are also used interchangeably herein and refer to binding molecules of antigen binding domains that specifically bind to an antigen, such as a polypeptide. A binding molecule or antigen binding domain that binds to or specifically binds to an antigen can be identified, for example, by immunoassays, Octet®, Biacore®, or other techniques known to those of skill in the art. In some embodiments, a binding molecule or antigen binding domain binds to or specifically binds to an antigen when it binds to an antigen with higher affinity than to any cross-reactive antigen as determined using experimental techniques, such as radioimmunoassay (RIA) and enzyme linked immunosorbent assay (ELISA). Typically, a specific or selective reaction will be at least twice background signal or noise and may be more than 10 times background. See, e.g., Fundamental Immunology 332-36 (Paul ed., 2d ed. 1989) for a discussion regarding binding specificity. In certain embodiments, the extent of binding of a binding molecule or antigen binding domain to a “non-target” protein is less than about 10% of the binding of the binding molecule or antigen binding domain to its particular target antigen, for example, as determined by FACS analysis or RIA. A binding molecule or antigen binding domain that binds to an antigen includes one that is capable of binding the antigen with sufficient affinity such that the binding molecule is useful, for example, as a therapeutic and/or diagnostic agent in targeting the antigen. In certain embodiments, a binding molecule or antigen binding domain that binds to an antigen has a dissociation constant (KD) of less than or equal to 1 μM, 800 nM, 600 nM, 550 nM, 500 nM, 300 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, a binding molecule or antigen binding domain binds to an epitope of an antigen that is conserved among the antigen from different species.

In certain embodiments, the binding molecules or antigen binding domains can comprise “chimeric” sequences in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., 1984, Proc. Natl. Acad. Sci. USA 81:6851-55). Chimeric sequences may include humanized sequences.

In certain embodiments, the binding molecules or antigen binding domains can comprise portions of “humanized” forms of nonhuman (e.g., camelid, murine, non-human primate) antibodies that include sequences from human immunoglobulins (e.g., recipient antibody) in which the native CDR residues are replaced by residues from the corresponding CDR of a nonhuman species (e.g., donor antibody) such as camelid, mouse, rat, rabbit, or nonhuman primate having the desired specificity, affinity, and capacity. In some instances, one or more FR region residues of the human immunoglobulin sequences are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., Nature 321:522-25 (1986); Riechmann et al., Nature 332:323-29 (1988); Presta, Curr. Op. Struct. Biol. 2:593-96 (1992); Carter et al., Proc. Natl. Acad. Sci. USA 89:4285-89 (1992); U.S. Pat. Nos. 6,800,738; 6,719,971; 6,639,055; 6,407,213; and 6,054,297.

In certain embodiments, the binding molecules or antigen binding domains can comprise portions of a “fully human antibody” or “human antibody,” wherein the terms are used interchangeably herein and refer to an antibody that comprises a human variable region and, for example, a human constant region. The binding molecules may comprise a single domain antibody sequence. In specific embodiments, the terms refer to an antibody that comprises a variable region and constant region of human origin. “Fully human” antibodies, in certain embodiments, can also encompass antibodies which bind polypeptides and are encoded by nucleic acid sequences which are naturally occurring somatic variants of human germline immunoglobulin nucleic acid sequence. The term “fully human antibody” includes antibodies having variable and constant regions corresponding to human germline immunoglobulin sequences as described by Kabat et al. (See Kabat et al. (1991) Sequences of Proteins of Immunological Interest, Fifth Edition, U.S. Department of Health and Human Services, NIH Publication No. 91-3242). A “human antibody” is one that possesses an amino acid sequence which corresponds to that of an antibody produced by a human and/or has been made using any of the techniques for making human antibodies. This definition of a human antibody specifically excludes a humanized antibody comprising non-human antigen-binding residues. Human antibodies can be produced using various techniques known in the art, including phage-display libraries (Hoogenboom and Winter, J. Mol. Biol. 227:381 (1991); Marks et al., J. Mol. Biol. 222:581 (1991)) and yeast display libraries (Chao et al., Nature Protocols 1: 755-68 (2006)). Also available for the preparation of human monoclonal antibodies are methods described in Cole et al., Monoclonal Antibodies and Cancer Therapy 77 (1985); Boerner et al., J. Immunol. 147(1):86-95 (1991); and van Dijk and van de Winkel, Curr. Opin. Pharmacol. 5: 368-74 (2001). Human antibodies can be prepared by administering the antigen to a transgenic animal that has been modified to produce such antibodies in response to antigenic challenge, but whose endogenous loci have been disabled, e.g., mice (see, e.g., Jakobovits, Curr. Opin. Biotechnol. 6(5):561-66 (1995); Brüggemann and Taussing, Curr. Opin. Biotechnol. 8(4):455-58 (1997); and U.S. Pat. Nos. 6,075,181 and 6,150,584 regarding XENOMOUSE™ technology). See also, for example, Li et al., Proc. Natl. Acad. Sci. USA 103:3557-62 (2006) regarding human antibodies generated via a human B-cell hybridoma technology.

In certain embodiments, the binding molecules or antigen binding domains can comprise portions of a “recombinant human antibody,” wherein the phrase includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see, e.g., Taylor, L. D. et al., Nucl. Acids Res. 20:6287-6295 (1992)) or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat, E. A. et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In certain embodiments, the binding molecules or antigen binding domains can comprise a portion of a “monoclonal antibody,” wherein the term as used herein refers to an antibody obtained from a population of substantially homogeneous antibodies, e.g., the individual antibodies comprising the population are identical except for possible naturally occurring mutations that may be present in minor amounts or well-known post-translational modifications such as amino acid isomerization or deamidation, methionine oxidation or asparagine or glutamine deamidation, each monoclonal antibody will typically recognize a single epitope on the antigen. In specific embodiments, a “monoclonal antibody,” as used herein, is an antibody produced by a single hybridoma or other cell. The term “monoclonal” is not limited to any particular method for making the antibody. For example, the monoclonal antibodies useful in the present disclosure may be prepared by the hybridoma methodology first described by Kohler et al., Nature 256:495 (1975), or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson et al., Nature 352:624-28 (1991) and Marks et al., J. Mol. Biol. 222:581-97 (1991), for example. Other methods for the preparation of clonal cell lines and of monoclonal antibodies expressed thereby are well known in the art. See, e.g., Short Protocols in Molecular Biology (Ausubel et al. eds., 5th ed. 2002).

A typical 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the a and 7 chains and four CH domains for and F isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites et al. eds., 8th ed. 1994); and Immunobiology (Janeway et al. eds., 5th ed. 2001).

The term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability according to the present disclosure. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders as described in more detail the sections below.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a R sheet configuration, connected by three hypervariable regions, which form loops connecting, and in some cases form part of, the R sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering according to Kabat” or “amino acid position numbering as in Kabat”, and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia, Contact, IMGT, and AHon.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: α, δ, and γ contain approximately 450 amino acids, while μ and ε contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains.

As used herein, the terms “hypervariable region,” “HVR,” “Complementarity Determining Region,” and “CDR” are used interchangeably. A “CDR” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. Accordingly, CDRs are variable region sequences interspersed within the framework region sequences.

CDR regions are well known to those skilled in the art and have been defined by well-known numbering systems. For example, the Kabat Complementarity Determining Regions (CDRs) are based on sequence variability and are the most commonly used (see, e.g., Kabat et al., supra). Chothia refers instead to the location of the structural loops (see, e.g., Chothia and Lesk, J. Mol. Biol. 196:901-17 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dübel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., Dev. Comp. Immunol. 27(1):55-77 (2003)). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plückthun, J. Mol. Biol. 309: 657-70 (2001). Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra). The residues from each of these hypervariable regions or CDRs are exemplified in Table 1 below.

TABLE 1
Exemplary CDRs According to Various Numbering Systems

| Loop | Kabat | AbM | Chothia | Contact | IMGT |
|---|---|---|---|---|---|
| CDR L1 | L24--L34 | L24--L34 | L26--L32 or | L30--L36 | L27--L38 |
| | | | L24--L34 | | |
| CDR L2 | L50--L56 | L50--L56 | L50--L52 or | L46--L55 | L56--L65 |
| | | | L50--L56 | | |
| CDR L3 | L89--L97 | L89--L97 | L91--L96 or | L89--L96 | L105-L117 |
| | | | L89--L97 | | |
| CDR H1 | H31--H35B | H26--H35B | H26--H32 . . . 34 | H30--H35B | H27--H38 |
| | (Kabat | | | | |
| | Numbering) | | | | |
| CDR H1 | H31--H35 | H26--H35 | H26--H32 | H30--H35 | |
| | (Chothia | | | | |
| | Numbering) | | | | |
| CDR H2 | H50--H65 | H50--H58 | H53--H55 or | H47--H58 | H56--H65 |
| | | | H52--H56 | | |
| CDR H3 | H95--H102 | H95--H102 | H96--H101 | H93--H101 | H105-H117 |
| | | | or | | |
| | | | H95--H102 | | |

The boundaries of a given CDR may vary depending on the scheme used for identification. Thus, unless otherwise specified, the terms “CDR” and “complementary determining region” of a given antibody or region thereof, such as a variable region, as well as individual CDRs (e.g., CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the IMGT, Kabat, Chothia, or Contact method. In other cases, the particular amino acid sequence of a CDR is given. It should be noted CDR regions may also be defined by a combination of various numbering systems, e.g., a combination of Kabat and Chothia numbering systems, or a combination of Kabat and IMGT numbering systems. Therefore, the term such as “a CDR as set forth in a specific VH” includes any CDR1 as defined by the exemplary CDR numbering systems described above, but is not limited thereby. Once a variable region (e.g., a VH or VL) is given, those skilled in the art would understand that CDRs within the region can be defined by different numbering systems or combinations thereof.

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule having a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “framework” or “FR” refers to those variable region residues flanking the CDRs. FR residues are present, for example, in chimeric, humanized, human, domain antibodies (e.g., single domain antibodies), diabodies, linear antibodies, and bispecific antibodies. FR residues are those variable domain residues other than the hypervariable region residues or CDR residues.

The term “Fe region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

As used herein, an “epitope” is a term in the art and refers to a localized region of an antigen to which a binding molecule (e.g., an antibody comprising a single domain antibody sequence) can specifically bind. An epitope can be a linear epitope or a conformational, non-linear, or discontinuous epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure. For example, in some embodiments, a binding molecule binds to a group of amino acids regardless of whether they are folded in a natural three dimensional protein structure. In other embodiments, a binding molecule requires amino acid residues making up the epitope to exhibit a particular conformation (e.g., bend, twist, turn or fold) in order to recognize and bind the epitope.

The term “specificity” refers to selective recognition of an antigen binding protein (such as an antibody) for a particular epitope of an antigen. Natural antibodies, for example, are monospecific. The term “multispecific” as used herein denotes that an antigen binding protein (such as an antibody) has two or more antigen-binding sites of which at least two bind different antigens. The term “bispecific” as used herein denotes that an antigen binding protein has two different antigen-binding specificities.

As used herein, the term “multispecific antibody” refers to an antibody that comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes do not overlap or do not substantially overlap. In an embodiment, the first and second epitopes are on different antigens, e.g., the different proteins (or different subunits of a multimeric protein). In an embodiment, a multispecific antibody comprises a third, fourth, or fifth immunoglobulin variable domain. In an embodiment, a multispecific antibody is a bispecific antibody molecule, a trispecific antibody molecule, or a tetraspecific antibody molecule.

As used herein, the term “bispecific antibody” refers to a multispecific antibody that binds no more than two epitopes or two antigens. A bispecific antibody is characterized by a first immunoglobulin variable domain sequence which has binding specificity for a first epitope (e.g., an epitope on a Vβ17 or TRBV19 antigen) and a second immunoglobulin variable domain sequence that has binding specificity for a second epitope. In an embodiment, the first and second epitopes are on different antigens, e.g., the different proteins (or different subunits of a multimeric protein). In an embodiment, a bispecific antibody comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody comprises a half antibody, or fragment thereof, having binding specificity for a first epitope and a half antibody, or fragment thereof, having binding specificity for a second epitope. In an embodiment, a bispecific antibody comprises a scFv, or fragment thereof, having binding specificity for a first epitope, and a scFv, or fragment thereof, having binding specificity for a second epitope. In an embodiment, the first epitope is located on Vβ17 and the second epitope is located on CD123. In an embodiment, the first epitope is located on Vβ17 and the second epitope is located on PD-1, PD-L1, CTLA-4, EGFR, HER-2, CD19, CD20, CD3 and/or other tumor associated immune suppressors or surface antigens. In an embodiment, the first epitope is located on Vβ17 and the second epitope is located on BCMA. In an embodiment, the first epitope is located on Vβ17 and the second epitope is located on DLL3. In an embodiment, the first epitope is located on Vβ17 and the second epitope is located on PSMA. In an embodiment, the first epitope is located on Vβ17 and the second epitope is located on KLK2. In an embodiment, the first epitope is located on TRBV19 and the second epitope is located on CD123. In an embodiment, the first epitope is located on TRBV19 and the second epitope is located on PD-1, PD-L1, CTLA-4, EGFR, HER-2, CD19, CD20, CD3 and/or other tumor associated immune suppressors or surface antigens. In an embodiment, the first epitope is located on TRBV19 and the second epitope is located on BCMA. In an embodiment, the first epitope is located on TRBV19 and the second epitope is located on DLL3. In an embodiment, the first epitope is located on TRBV19 and the second epitope is located on PSMA. In an embodiment, the first epitope is located on TRBV19 and the second epitope is located on KLK2.

As used herein, the term “Vβ17” refers to a T cell receptor, which is expressed in response to an immune response on a cytotoxic T cell. Vβ17-expressing CD8+ T cells are commonly produced in response to influenza A virus exposure in a subject. Vβ17-expressing CD8+ T cells provide great recall in response to influenza exposure in the subject. The term “Vβ17” includes any Vβ17 variant, isoform, and species homolog, which is naturally expressed by cells (including T cells) or can be expressed on cells transfected with genes or cDNA encoding the polypeptide. Unless noted, preferably the Vβ17 is a human Vβ17. An exemplary human Vβ17 amino acid sequence is provided by GenBank Accession Number AAB49730.1.

As used herein, the term “T Cell Receptor Beta Variable 19 (TRBV19)” refers to V region of the variable domain of T cell receptor (TR) beta chain that participates in the antigen recognition. The term “TRBV19” includes any TRBV19 variant, isoform, and species homolog, which is naturally expressed by cells (including T cells) or can be expressed on cells transfected with genes or cDNA encoding the polypeptide. Unless noted, preferably the TRBV19 is a human TRBV19.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies or other members of the immunoglobulin superfamily, in certain embodiments, a “polypeptide” can occur as a single chain or as two or more associated chains.

“Polynucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. A cell that produces a binding molecule of the present disclosure may include a parent hybridoma cell, as well as bacterial and eukaryotic host cells into which nucleic acids encoding the antibodies have been introduced. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

An “isolated nucleic acid” is a nucleic acid, for example, an RNA, DNA, or a mixed nucleic acids, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, one or more nucleic acid molecules encoding a single domain antibody or an antibody as described herein are isolated or purified. The term embraces nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

As used herein, the term “operatively linked,” and similar phrases (e.g., genetically fused), when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

The term “vector” refers to a substance that is used to carry or include a nucleic acid sequence, including for example, a nucleic acid sequence encoding a binding molecule (e.g., an antibody) as described herein, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed (e.g., both an antibody heavy and light chain or an antibody VH and VL), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product and it is further understood that expression levels can be optimized to obtain sufficient expression using methods well known in the art.

The term “host” as used herein refers to an animal, such as a mammal (e.g., a human).

The term “host cell” as used herein refers to a particular subject cell that may be transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

As used herein, the term “autologous” is meant to refer to any material derived from the same individual to whom it is later to be re-introduced into the individual.

As used herein, the term “allogeneic” refers to a graft derived from a different individual of the same species.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

As used herein, the term “isolation” or “isolating” refers to a process of increasing the percentage of a certain substance in a composition. For example, isolating a type of cells from a population of cells refers to a process of creating a population of cells in which the percentage of this type of cells increases as compared to the percentage of this type of cells in the original population of cells. Therefore, the term “isolated” when used in the context of a type of cells does not mean that the isolated population of cells comprises 100% of this type of cells, rather it means the percentage of this type of cells increases in a population of cells after the isolation process.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

“Excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete) or vehicle.

In some embodiments, excipients are pharmaceutically acceptable excipients. Examples of pharmaceutically acceptable excipients include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. Other examples of pharmaceutically acceptable excipients are described in Remington and Gennaro, Remington's Pharmaceutical Sciences (18th ed. 1990).

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

In some embodiments, excipients are sterile liquids, such as water and oils, including those of petroleum, animal, vegetable, or synthetic origin, such as peanut oil, soybean oil, mineral oil, sesame oil, and the like. Water is an exemplary excipient when a composition (e.g., a pharmaceutical composition) is administered intravenously. Saline solutions and aqueous dextrose and glycerol solutions can also be employed as liquid excipients, particularly for injectable solutions. An excipient can also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard excipients such as pharmaceutical grades of mannitol, lactose, starch, magnesium stearate, sodium saccharine, cellulose, magnesium carbonate, etc.

Compositions, including pharmaceutical compounds, may contain a binding molecule (e.g., an antibody), for example, in isolated or purified form, together with a suitable amount of excipients.

The term “effective amount” or “therapeutically effective amount” as used herein refers to the amount of a single domain antibody or a therapeutic molecule comprising an agent and the single domain antibody or pharmaceutical composition provided herein which is sufficient to result in the desired outcome.

The terms “subject” and “patient” may be used interchangeably. As used herein, in certain embodiments, a subject is a mammal, such as a non-primate or a primate (e.g., human). In specific embodiments, the subject is a human. In one embodiment, the subject is a mammal, e.g., a human, diagnosed with a disease or disorder. In another embodiment, the subject is a mammal, e.g., a human, at risk of developing a disease or disorder.

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease.

The terms “prevent,” “preventing,” and “prevention” refer to reducing the likelihood of the onset (or recurrence) of a disease, disorder, condition, or associated symptom(s) (e.g., diabetes or a cancer).

The terms “about” and “approximately” mean within 20%, within 15%, within 10%, within 9%, within 8%, within 7%, within 6%, within 5%, within 4%, within 3%, within 2%, within 1%, or less of a given value or range.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### 5.2. Methods for Producing Influenza A Virus Specific Vβ17⁺CD8⁺ T Cells

Provided herein, in one aspect, is a method for activating or enriching Vβ17⁺CD8⁺ T cells that are specific to influenza A virus. In some embodiments, the methods provided herein comprises contacting a population of cells comprising T cells with a peptide derived from human influenza A virus.

### 5.2.1. Obtaining a Population of Cells Comprising T Cells

Exemplary T cells include CD8⁺ T cells, CD4⁺ T cells, regulatory T cells, cytotoxic T cells, and tumor infiltrating lymphocytes. T cells can be obtained from a number of sources. In some embodiments, the population of cells comprising T cells is collected, isolated, purified or induced from a body fluid, a tissue or an organ including but not limited to peripheral blood, umbilical cord blood, bone marrow, lymph node, the thymus, spleen, or other tissues or fluids of a mammal. In other embodiments, the population of cells comprising T cells is obtained from a cultured T-cell line. In specific embodiments, the population of cells comprising T cells is peripheral blood lymphocytes, precursor cells of T cells (such as hematopoietic stem cells, lymphocyte precursor cells etc.) or a cell population containing them. In a specific embodiment, the population of cells comprising T cells is PBMCs. In some embodiments, the PBMCs are from a healthy donor. In some embodiments, the PBMCs are from an unhealthy donor. Various methods of collecting and preparing PBMCs are known in the art.

In some embodiments, T cells can be obtained from a unit of blood collected from a subject using any number of techniques known to the skilled artisan, such as Ficoll™ separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In some embodiments, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or other saline solution with or without buffer. Alternatively, the undesirable components of the apheresis sample may be removed and the cells directly resuspended in culture media.

In some embodiments, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3+, CD28+, CD4+, CD8+, CD45RA+, and CD45RO+ T cells, can be further isolated by positive or negative selection techniques.

For example, in some embodiments, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In some embodiments, the time period is at least 0.5, 1, 2, 3, 4, 5, or 6 hours. In some embodiments, the time period ranges from 0.5 to 36 hours or longer and all integer values there between. In some embodiments, the time period is 1 to 36 hours. In some embodiments, the time period is 2 to 36 hours. In some embodiments, the time period is 3 to 36 hours. In some embodiments, the time period is 4 to 36 hours. In some embodiments, the time period is 5 to 36 hours. In some embodiments, the time period is 6 to 24 hours. In some embodiments, the time period is 7 to 24 hours. In some embodiments, the time period is 8 to 24 hours. In some embodiments, the time period is 9 to 24 hours. In some embodiments, the time period is 10 to 24 hours. In some embodiments, the incubation time period is 0.5 hours. In some embodiments, the incubation time period is 1 hours. In some embodiments, the incubation time period is 2 hours. In some embodiments, the incubation time period is 3 hours. In some embodiments, the incubation time period is 4 hours. In some embodiments, the incubation time period is 5 hours. In some embodiments, the incubation time period is 6 hours. In some embodiments, the incubation time period is 7 hours. In some embodiments, the incubation time period is 8 hours. In some embodiments, the incubation time period is 9 hours. In some embodiments, the incubation time period is 10 hours. In some embodiments, the incubation time period is 11 hours. In some embodiments, the incubation time period is 12 hours. In some embodiments, the incubation time period is 13 hours. In some embodiments, the incubation time period is 14 hours. In some embodiments, the incubation time period is 15 hours. In some embodiments, the incubation time period is 16 hours. In some embodiments, the incubation time period is 17 hours. In some embodiments, the incubation time period is 18 hours. In some embodiments, the incubation time period is 19 hours. In some embodiments, the incubation time period is 20 hours. In some embodiments, the incubation time period is 21 hours. In some embodiments, the incubation time period is 22 hours. In some embodiments, the incubation time period is 23 hours. In some embodiments, the incubation time period is 24 hours.

Longer incubation times may be used to isolate T cells in any situation where there are few T cells as compared to other cell types. Further, use of longer incubation times can increase the efficiency of capture of CD8+ T cells. Thus, in some embodiments, by simply shortening or lengthening the time T cells are allowed to bind to the CD3/CD28 beads and/or by increasing or decreasing the ratio of beads to T cells, subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used. In some embodiments, it may be desirable to perform the selection procedure and use the “unselected” cells in the present methods or processes. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. In some embodiments, the concentration of cells ranges from 10 million cells/ml to 5 billion cells/ml. In some embodiments, the concentration of cells ranges from 15 million cells/ml to 4 billion cells/ml. In some embodiments, the concentration of cells ranges from 20 million cells/ml to 3 billion cells/ml. In some embodiments, the concentration of cells ranges from 25 million cells/ml to 2 billion cells/ml. In some embodiments, the concentration of cells ranges from 30 million cells/ml to 2 billion cells/ml. In some embodiments, the concentration of cells ranges from 35 million cells/ml to 2 billion cells/ml. In some embodiments, the concentration of cells ranges from 40 million cells/ml to 2 billion cells/ml. In some embodiments, the concentration of cells ranges from 45 million cells/ml to 2 billion cells/ml. In some embodiments, the concentration of cells ranges from 50 million cells/ml to 2 billion cells/ml. In some embodiments, a concentration of cells of 10, 15, 20, 25, 30, 35, 40, 45, or 50 million cells/ml is used. In some embodiments, a concentration of cells of 55, 60, 65, 70, 75, 80, 85, 90, 95, or 100 million cells/ml is used. In some embodiments, greater than 100 million cells/ml is used. In some embodiments, concentrations of 125 or 150 million cells/ml can be used. In some embodiments, a concentration of 1 billion cells/ml is used. In some embodiments, a concentration of 2 billion cells/ml is used. Using high concentrations may result in increased cell yield, cell activation, and cell expansion. Further, use of high cell concentrations may allow more efficient capture of cells that may weakly express target antigens of interest. In some embodiments, using high concentration of cells allows more efficient selection of CD8+ T cells that normally have weaker CD28 expression.

In some embodiments, the cells may be incubated on a rotator for varying lengths of time at varying speeds at either 2-10° C., or at room temperature. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% dextran 40 and 5% dextrose, 20% human serum albumin and 7.5% DMSO, or 31.25% plasmalyte-A, 31.25% dextrose 5%, 0.45% NaCl, 10% dextran 40 and 5% dextrose, 20% human serum albumin, and 7.5% DMSO or other suitable cell freezing media containing for example, Hespan and PlasmaLyte A. The cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In some embodiments, cryopreserved cells are thawed and washed as described herein and allowed to rest for one hour at room temperature prior to activation.

In certain embodiments, provided in the present disclosure is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one embodiment, a blood sample or an apheresis is taken from a generally healthy subject. In certain embodiments, the T cells may be expanded, frozen, and used at a later time.

### 5.2.2. M1 Peptide Stimulation

Provided herein, in one aspect, is a method comprising activating or enriching Vβ17⁺CD8⁺ T cells by contacting a peptide from matrix protein derived from human influenza A virus with a population of cells comprising T cells provided herein.

In some embodiments, the peptide from matrix protein derived from human influenza A virus is a M1 peptide derived from human influenza A virus (M1₅₈₋₆₆) comprising the amino acid sequence of GILGFVFTL (SEQ ID NO:1).

In other embodiments, the peptide from matrix protein derived from human influenza A virus is peptide 57-68 (KGILGFVFTLTV (SEQ ID NO:2)). In other embodiments, the peptide from matrix protein derived from human influenza A virus is peptide 57-67 (KGILGFVFTLT (SEQ ID NO:3)). In other embodiments, the peptide from matrix protein derived from human influenza A virus is peptide 57-66 (KGILGFVFTL (SEQ ID NO:4)). In other embodiments, the peptide from matrix protein derived from human influenza A virus is peptide 58-68 (GILGFVFTLTV (SEQ ID NO:5)). In other embodiments, the peptide from matrix protein derived from human influenza A virus is peptide 58-67 (GILGFVFTLT (SEQ ID NO:6)).

In some embodiments, the method provided herein comprises contacting the M1 peptide derived from human influenza A virus (M1₅₈₋₆₆) with the population of cells comprising T cells with a certain M1 peptide to cell ratio, for example, 1 to 10 μg/ml of M1 peptide with 10³ to 10¹¹ cells. In one embodiment, the M1 peptide to cell ratio is 1 μg/ml of M1 peptide with about 2.5×10³ cells. In one embodiment, the M1 peptide to cell ratio is 1 μg/ml of M1 peptide with 2.5×10⁴ cells. In one embodiment, the M1 peptide to cell ratio is 1 μg/ml of M1 peptide with 2.5×10⁵ cells. In one embodiment, the M1 peptide to cell ratio is 1 μg/ml of M1 peptide with 2.5×10⁶ cells. In one embodiment, the M1 peptide to cell ratio is 1 μg/ml of M1 peptide with 2.5×10⁷ cells. In one embodiment, the M1 peptide to cell ratio is 1 μg/ml of M1 peptide with 2.5×10⁸ cells. In one embodiment, the M1 peptide to cell ratio is 1 μg/ml of M1 peptide with 2.5×10⁹ cells. In one embodiment, the M1 peptide to cell ratio is 1 μg/ml of M1 peptide with 2.5×10¹⁰ cells.

In some embodiments, the method provided herein comprises contacting the M1 peptide derived from human influenza A virus (M1₅₈₋₆₆) with the population of cells comprising T cells for certain amount of time. In some embodiments, the M1 peptide contacts the cells for 1 to 20 days. In some embodiments, the M1 peptide contacts the cells for 2 to 18 days. In some embodiments, the M1 peptide contacts the cells for 3 to 16 days. In some embodiments, the M1 peptide contacts the cells for 4 to 14 days. In some embodiments, the M1 peptide contacts the cells for 5 to 14 days. In some embodiments, the M1 peptide contacts the cells for 6 to 14 days. In some embodiments, the M1 peptide contacts the cells for 7 to 14 days. In some embodiments, the M1 peptide contacts the cells for 8 to 14 days. In some embodiments, the M1 peptide contacts the cells for 9 to 14 days. In some embodiments, the M1 peptide contacts the cells for 10 to 14 days. In some embodiments, the M1 peptide contacts the cells for 11 to 14 days. In some embodiments, the M1 peptide contacts the cells for 12 to 14 days. In one embodiment, the M1 peptide contacts the cells for 1 day. In one embodiment, the M1 peptide contacts the cells for 2 days. In one embodiment, the M1 peptide contacts the cells for 3 days. In one embodiment, the M1 peptide contacts the cells for 4 days. In one embodiment, the M1 peptide contacts the cells for 5 days. In one embodiment, the M1 peptide contacts the cells for 6 days. In one embodiment, the M1 peptide contacts the cells for 7 days. In one embodiment, the M1 peptide contacts the cells for 8 days. In one embodiment, the M1 peptide contacts the cells for 9 days. In one embodiment, the M1 peptide contacts the cells for 10 days. In one embodiment, the M1 peptide contacts the cells for 11 days. In one embodiment, the M1 peptide contacts the cells for 12 days. In one embodiment, the M1 peptide contacts the cells for 13 days. In one embodiment, the M1 peptide contacts the cells for 14 days.

In some embodiments, the method provided herein further comprises contacting an agent of immunostimulation or immunoregulation with the population of the cells comprising the T cells that have been contacted with the M1 peptide. In some specific embodiments, the agent of immunostimulation or immunoregulation is able to stimulate T cells. In a preferred embodiment, the agent of immunostimulation or immunoregulation is IL-2.

In some embodiments, the method provided herein further comprises contacting IL-2 with the population of the cells comprising the T cells with a certain IL-2 to cell ratio, e.g., 100 to 250 IU of IL-2 with 10³ to 10¹¹ cells. In one embodiment, the IL-2 to cell ratio is 210 IU of IL-2 with 2.5×10³ cells. In one embodiment, the IL-2 to cell ratio is 210 IU of IL-2 with 2.5×10⁴ cells. In one embodiment, the IL-2 to cell ratio is 210 IU of IL-2 with 2.5×10⁵ cells. In one embodiment, the IL-2 to cell ratio is 210 IU of IL-2 with 2.5×10⁶ cells. In one embodiment, the IL-2 to cell ratio is 210 IU of IL-2 with 2.5×10⁷ cells. In one embodiment, the IL-2 to cell ratio is 210 IU of IL-2 with 2.5×10⁸ cells. In one embodiment, the IL-2 to cell ratio is 210 IU of IL-2 with 2.5×10⁹ cells. In one embodiment, the IL-2 to cell ratio is 210 IU of IL-2 with 2.5×10¹⁰ cells.

In some embodiments, the method provided herein further comprises contacting IL-2 with the population of the cells comprising the T cells for certain amount of time. In some embodiments, the cells are contacted with IL-2 for 1 to 20 days. In some embodiments, the cells are contacted with IL-2 for 2 to 18 days. In some embodiments, the cells are contacted with IL-2 for 3 to 16 days. In some embodiments, the cells are contacted with IL-2 for 4 to 14 days. In some embodiments, the cells are contacted with IL-2 for 5 to 14 days. In some embodiments, the cells are contacted with IL-2 for 6 to 14 days. In some embodiments, the cells are contacted with IL-2 for 7 to 14 days. In some embodiments, the cells are contacted with IL-2 for 8 to 14 days. In some embodiments, the cells are contacted with IL-2 for 9 to 14 days. In some embodiments, the cells are contacted with IL-2 for 10 to 14 days. In some embodiments, the cells are contacted with IL-2 for 11 to 14 days. In some embodiments, the cells are contacted with IL-2 for 12 to 14 days. In one embodiment, the cells are contacted with IL-2 for 1 day. In one embodiment, the cells are contacted with IL-2 for 2 days. In one embodiment, the cells are contacted with IL-2 for 3 days. In one embodiment, the cells are contacted with IL-2 for 4 days. In one embodiment, the cells are contacted with IL-2 for 5 days. In one embodiment, the cells are contacted with IL-2 for 6 days. In one embodiment, the cells are contacted with IL-2 for 7 days. In one embodiment, the cells are contacted with IL-2 for 8 days. In one embodiment, the cells are contacted with IL-2 for 9 days. In one embodiment, the cells are contacted with IL-2 for 10 days. In one embodiment, the cells are contacted with IL-2 for 11 days. In one embodiment, the cells are contacted with IL-2 for 12 days. In one embodiment, the cells are contacted with IL-2 for 13 days. In one embodiment, the cells are contacted with IL-2 for 14 days.

In certain embodiments, the population of the cells comprising T cells provided herein are contacted with the M1 peptide and/or IL-2 ex vivo, e.g., in a culture medium. In some embodiments, the method comprises culturing the population of the cells ex vivo in a medium comprising the M1 peptide and IL-2 for a period of time, e.g., for 5 to 20 days.

In other embodiments, the method comprises culturing the population of the cells ex vivo in a medium comprising the M1 peptide for a period of time, e.g., for 5 to 10 days, and then culturing the population of the cells ex vivo in a medium comprising IL-2 for a period of time, e.g., for another 5 to 10 days.

In yet other embodiments, the method comprises culturing the population of the cells ex vivo in a medium comprising the M1 peptide, then culturing the population of the cells ex vivo in a medium comprising the M1 peptide and IL-2; and then culturing the population of the cells ex vivo in a medium comprising IL-2. For example, the population of the cells is first cultured ex vivo in a medium comprising the M1 peptide for a period of time, and then IL-2 is added into the medium for a period of time. Then the cells are washed and cultured again in a medium comprising IL-2. In a specific embodiment, the population of the cells is first cultured ex vivo in a medium comprising the M1 peptide for 1 to 3 days before IL-2 is added into the medium; the population of the cells is then cultured ex vivo in the medium comprising both M1 peptide and IL-2 for 1 to 7 days; and then the cells are washed and cultured in a medium comprising IL-2 for 1 to 10 days. In another specific embodiment, the population of the cells comprising the T cells are cultured using a method as illustrated in Section 7 below.

In some embodiments, the amount of a specific type of cells is measured by methods well known to those skilled in the art. In some embodiments, the amount of a specific type of cells is measured by flow cytometry analysis.

In some embodiments, the initial percent of the Vβ17⁺CD8⁺ T cells in the CD8+ cells from the population of the cells is 2-10% of the CD8+ cells. In some embodiments, the initial percent of the Vβ17⁺CD8⁺ T cells in the CD8+ cells from the population of the cells is 3-8% of the CD8+ cells. In some embodiments, the initial percent of the Vβ17⁺CD8⁺ T cells in the CD8+ cells from the population of the cells is 4-6% of the CD8+ cells. In some embodiments, the initial percent of the Vβ17⁺CD8⁺ T cells in the CD8+ cells from the population of the cells is 4-5% of the CD8+ cells. In some embodiments, the initial percent of the Vβ17⁺CD8⁺ T cells in the CD8+ cells from the population of the cells is 5-6% of the CD8+ cells. In some embodiments, the initial percent of the Vβ17⁺CD8⁺ T cells in the CD8+ cells from the population of the cells is 5.5% of the CD8+ cells.

In some embodiments, the method provided herein increases the percentage of the Vβ17⁺CD8⁺ T cells in the CD8+ cells from the population of the cells by at least 10% of the initial percent of the Vβ17⁺CD8⁺ T cells in the CD8+ cells. In other embodiments, the method provided herein increases the percentage of the Vβ17⁺CD8⁺ T cells in the CD8+ cells from the population of the cells by at least 20% of the initial percent of the Vβ17⁺CD8⁺ T cells in the CD8+ cells. In other embodiments, the method provided herein increases the percentage of the Vβ17⁺CD8⁺ T cells in the CD8+ cells from the population of the cells by at least 30% of the initial percent of the Vβ17⁺CD8⁺ T cells in the CD8+ cells. In other embodiments, the method provided herein increases the percentage of the Vβ17⁺CD8⁺ T cells in the CD8+ cells from the population of the cells by at least 40% of the initial percent of the Vβ17⁺CD8⁺ T cells in the CD8+ cells. In other embodiments, the method provided herein increases the percentage of the Vβ17⁺CD8⁺ T cells in the CD8+ cells from the population of the cells by at least 50% of the initial percent of the Vβ17⁺CD8⁺ T cells in the CD8+ cells. In other embodiments, the method provided herein increases the percentage of the Vβ17⁺CD8⁺ T cells in the CD8+ cells from the population of the cells by at least 60% of the initial percent of the Vβ17⁺CD8⁺ T cells in the CD8+ cells. In other embodiments, the method provided herein increases the percentage of the Vβ17⁺CD8⁺ T cells in the CD8+ cells from the population of the cells by at least 70% of the initial percent of the Vβ17⁺CD8⁺ T cells in the CD8+ cells. In other embodiments, the method provided herein increases the percentage of the Vβ17⁺CD8⁺ T cells in the CD8+ cells from the population of the cells by at least 80% of the initial percent of the Vβ17⁺CD8⁺ T cells in the CD8+ cells. In other embodiments, the method provided herein increases the percentage of the Vβ17⁺CD8⁺ T cells in the CD8+ cells from the population of the cells by at least 90% of the initial percent of the Vβ17⁺CD8⁺ T cells in the CD8+ cells. In other embodiments, the method provided herein increases the percentage of the Vβ17⁺CD8⁺ T cells in the CD8+ cells from the population of the cells by at least 2 fold of the initial percent of the Vβ17⁺CD8⁺ T cells in the CD8+ cells. In other embodiments, the method provided herein increases the percentage of the Vβ17⁺CD8⁺ T cells in the CD8+ cells from the population of the cells by at least 3 fold of the initial percent of the Vβ17⁺CD8⁺ T cells in the CD8+ cells. In other embodiments, the method provided herein increases the percentage of the Vβ17⁺CD8⁺ T cells in the CD8+ cells from the population of the cells by at least 4 fold of the initial percent of the Vβ17⁺CD8⁺ T cells in the CD8+ cells. In other embodiments, the method provided herein increases the percentage of the Vβ17⁺CD8⁺ T cells in the CD8+ cells from the population of the cells by at least 5 fold of the initial percent of the Vβ17⁺CD8⁺ T cells in the CD8+ cells. In other embodiments, the method provided herein increases the percentage of the Vβ17⁺CD8⁺ T cells in the CD8+ cells from the population of the cells by at least 6 fold of the initial percent of the Vβ17⁺CD8⁺ T cells in the CD8+ cells. In other embodiments, the method provided herein increases the percentage of the Vβ17⁺CD8⁺ T cells in the CD8+ cells from the population of the cells by at least 7 fold of the initial percent of the Vβ17⁺CD8⁺ T cells in the CD8+ cells. In other embodiments, the method provided herein increases the percentage of the Vβ17⁺CD8⁺ T cells in the CD8+ cells from the population of the cells by at least 8 fold of the initial percent of the Vβ17⁺CD8⁺ T cells in the CD8+ cells. In other embodiments, the method provided herein increases the percentage of the Vβ17⁺CD8⁺ T cells in the CD8+ cells from the population of the cells by at least 9 fold of the initial percent of the Vβ17⁺CD8⁺ T cells in the CD8+ cells. In other embodiments, the method provided herein increases the percentage of the Vβ17⁺CD8⁺ T cells in the CD8+ cells from the population of the cells by at least 10 fold of the initial percent of the Vβ17⁺CD8⁺ T cells in the CD8+ cells. In other embodiments, the method provided herein increases the percentage of the Vβ17⁺CD8⁺ T cells in the CD8⁺ cells from the population of the cells by at least 11 fold of the initial percent of the Vβ17⁺CD8⁺ T cells in the CD8+ cells. In other embodiments, the method provided herein increases the percentage of the Vβ17⁺CD8⁺ T cells in the CD8+ cells from the population of the cells by at least 12 fold of the initial percent of the Vβ17⁺CD8⁺ T cells in the CD8+ cells. In other embodiments, the method provided herein increases the percentage of the Vβ17⁺CD8⁺ T cells in the CD8+ cells from the population of the cells by at least 13 fold of the initial percent of the Vβ17⁺CD8⁺ T cells in the CD8+ cells. In other embodiments, the method provided herein increases the percentage of the Vβ17⁺CD8⁺ T cells in the CD8+ cells from the population of the cells by at least 14 fold of the initial percent of the Vβ17⁺CD8⁺ T cells in the CD8+ cells. In other embodiments, the method provided herein increases the percentage of the Vβ17⁺CD8⁺ T cells in the CD8+ cells from the population of the cells by at least 15 fold of the initial percent of the Vβ17⁺CD8⁺ T cells in the CD8+ cells.

In some embodiments, the method provided herein increases the percentage of the Vβ17⁺CD8⁺ T cells in the CD8+ cells from the population of the cells by 10% to 20 fold of the initial percent of the Vβ17⁺CD8⁺ T cells in the CD8+ cells. In some embodiments, the method provided herein increases the percentage of the Vβ17⁺CD8⁺ T cells in the CD8+ cells from the population of the cells by 20% to 20 fold of the initial percent of the Vβ17⁺CD8⁺ T cells in the CD8+ cells. In some embodiments, the method provided herein increases the percentage of the Vβ17⁺CD8⁺ T cells in the CD8+ cells from the population of the cells by 30% to 20 fold of the initial percent of the Vβ17⁺CD8⁺ T cells in the CD8+ cells. In some embodiments, the method provided herein increases the percentage of the Vβ17⁺CD8⁺ T cells in the CD8+ cells from the population of the cells by 40% to 20 fold of the initial percent of the Vβ17⁺CD8⁺ T cells in the CD8+ cells. In some embodiments, the method provided herein increases the percentage of the Vβ17⁺CD8⁺ T cells in the CD8+ cells from the population of the cells by 50% to 20 fold of the initial percent of the Vβ17⁺CD8⁺ T cells in the CD8+ cells. In some embodiments, the method provided herein increases the percentage of the Vβ17⁺CD8⁺ T cells in the CD8+ cells from the population of the cells by 60% to 20 fold of the initial percent of the Vβ17⁺CD8⁺ T cells in the CD8+ cells. In some embodiments, the method provided herein increases the percentage of the Vβ17⁺CD8⁺ T cells in the CD8+ cells from the population of the cells by 70% to 20 fold of the initial percent of the Vβ17⁺CD8⁺ T cells in the CD8+ cells. In some embodiments, the method provided herein increases the percentage of the Vβ17⁺CD8⁺ T cells in the CD8+ cells from the population of the cells by 80% to 20 fold of the initial percent of the Vβ17⁺CD8⁺ T cells in the CD8+ cells. In some embodiments, the method provided herein increases the percentage of the Vβ17⁺CD8⁺ T cells in the CD8+ cells from the population of the cells by 90% to 20 fold of the initial percent of the Vβ17⁺CD8⁺ T cells in the CD8+ cells. In some embodiments, the method provided herein increases the percentage of the Vβ17⁺CD8⁺ T cells in the CD8+ cells from the population of the cells by 2 to 20 fold of the initial percent of the Vβ17⁺CD8⁺ T cells in the CD8+ cells. In some embodiments, the method provided herein increases the percentage of the Vβ17⁺CD8⁺ T cells in the CD8+ cells from the population of the cells by 3 to 20 fold of the initial percent of the Vβ17⁺CD8⁺ T cells in the CD8+ cells. In some embodiments, the method provided herein increases the percentage of the Vβ17⁺CD8⁺ T cells in the CD8+ cells from the population of the cells by 4 to 20 fold of the initial percent of the Vβ17⁺CD8⁺ T cells in the CD8+ cells. In some embodiments, the method provided herein increases the percentage of the Vβ17⁺CD8⁺ T cells in the CD8+ cells from the population of the cells by 5 to 20 fold of the initial percent of the Vβ17⁺CD8⁺ T cells in the CD8+ cells. In some embodiments, the method provided herein increases the percentage of the Vβ17⁺CD8⁺ T cells in the CD8+ cells from the population of the cells by 6 to 20 fold of the initial percent of the Vβ17⁺CD8⁺ T cells in the CD8+ cells. In some embodiments, the method provided herein increases the percentage of the Vβ17⁺CD8⁺ T cells in the CD8+ cells from the population of the cells by 7 to 20 fold of the initial percent of the Vβ17⁺CD8⁺ T cells in the CD8+ cells. In some embodiments, the method provided herein increases the percentage of the Vβ17⁺CD8⁺ T cells in the CD8+ cells from the population of the cells by 8 to 20 fold of the initial percent of the Vβ17⁺CD8⁺ T cells in the CD8+ cells. In some embodiments, the method provided herein increases the percentage of the Vβ17⁺CD8⁺ T cells in the CD8+ cells from the population of the cells by 9 to 20 fold of the initial percent of the Vβ17⁺CD8⁺ T cells in the CD8+ cells. In some embodiments, the method provided herein increases the percentage of the Vβ17⁺CD8⁺ T cells in the CD8+ cells from the population of the cells by 10 to 20 fold of the initial percent of the Vβ17⁺CD8⁺ T cells in the CD8+ cells. In some embodiments, the method provided herein increases the percentage of the Vβ17⁺CD8⁺ T cells in the CD8+ cells from the population of the cells by 11 to 20 fold of the initial percent of the Vβ17⁺CD8⁺ T cells in the CD8+ cells. In some embodiments, the method provided herein increases the percentage of the Vβ17⁺CD8⁺ T cells in the CD8+ cells from the population of the cells by 12 to 20 fold of the initial percent of the Vβ17⁺CD8⁺ T cells in the CD8+ cells. In some embodiments, the method provided herein increases the percentage of the Vβ17⁺CD8⁺ T cells in the CD8+ cells from the population of the cells by 13 to 20 fold of the initial percent of the Vβ17⁺CD8⁺ T cells in the CD8+ cells. In some embodiments, the method provided herein increases the percentage of the Vβ17⁺CD8⁺ T cells in the CD8+ cells from the population of the cells by 14 to 20 fold of the initial percent of the Vβ17⁺CD8⁺ T cells in the CD8+ cells. In some embodiments, the method provided herein increases the percentage of the Vβ17⁺CD8⁺ T cells in the CD8+ cells from the population of the cells by 15 to 20 fold of the initial percent of the Vβ17⁺CD8⁺ T cells in the CD8+ cells.

The baseline percentage of Vβ17⁺CD8⁺ T cells among CD8⁺ T cells among whole PBMCs of healthy individuals is about 5.5% of the CD8⁺ cells. In some embodiments, after contacting the population of cells comprising T cells with the M1 peptide derived from human influenza A virus (M1₅₈₋₆₆) and/or IL-2, the percentage of Vβ17⁺CD8⁺ T cells among CD8⁺ T cells is increased.

In some embodiments, the percentage of Vβ17⁺CD8⁺ T cells among CD8⁺ T cells after being contacted with the M1 peptide and/or IL-2 is at least 6% of the CD8⁺ cells. In some embodiments, the percentage of Vβ17⁺CD8⁺ T cells among CD8⁺ T cells after being contacted with the M1 peptide and/or IL-2 is at least 10% of the CD8⁺ cells. In some embodiments, the percentage of Vβ17⁺CD8⁺ T cells among CD8⁺ T cells after being contacted with the M1 peptide and/or IL-2 is at least 15% of the CD8⁺ cells. In some embodiments, the percentage of Vβ17⁺CD8⁺ T cells among CD8⁺ T cells after being contacted with the M1 peptide and/or IL-2 is at least 20% of the CD8⁺ cells. In some embodiments, the percentage of Vβ17⁺CD8⁺ T cells among CD8⁺ T cells after being contacted with the M1 peptide and/or IL-2 is at least 25% of the CD8⁺ cells. In some embodiments, the percentage of Vβ17⁺CD8⁺ T cells among CD8⁺ T cells after being contacted with the M1 peptide and/or IL-2 is at least 30% of the CD8⁺ cells. In some embodiments, the percentage of Vβ17⁺CD8⁺ T cells among CD8⁺ T cells after being contacted with the M1 peptide and/or IL-2 is at least 35% of the CD8⁺ cells. In some embodiments, the percentage of Vβ17⁺CD8⁺ T cells among CD8⁺ T cells after being contacted with the M1 peptide and/or IL-2 is at least 40% of the CD8⁺ cells. In some embodiments, the percentage of Vβ17⁺CD8⁺ T cells among CD8⁺ T cells after being contacted with the M1 peptide and/or IL-2 is at least 45% of the CD8⁺ cells. In some embodiments, the percentage of Vβ17⁺CD8⁺ T cells among CD8⁺ T cells after being contacted with the M1 peptide and/or IL-2 is at least 50% of the CD8⁺ cells. In some embodiments, the percentage of Vβ17⁺CD8⁺ T cells among CD8⁺ T cells after being contacted with the M1 peptide and/or IL-2 is at least 55% of the CD8⁺ cells. In some embodiments, the percentage of Vβ17⁺CD8⁺ T cells among CD8⁺ T cells after being contacted with the M1 peptide and/or IL-2 is at least 60% of the CD8⁺ cells. In some embodiments, the percentage of Vβ17⁺CD8⁺ T cells among CD8⁺ T cells after being contacted with the M1 peptide and/or IL-2 is at least 65% of the CD8⁺ cells. In some embodiments, the percentage of Vβ17⁺CD8⁺ T cells among CD8⁺ T cells after being contacted with the M1 peptide and/or IL-2 is at least 70% of the CD8⁺ cells. In some embodiments, the percentage of Vβ17⁺CD8⁺ T cells among CD8⁺ T cells after being contacted with the M1 peptide and/or IL-2 is at least 75% of the CD8⁺ cells. In some embodiments, the percentage of Vβ17⁺CD8⁺ T cells among CD8⁺ T cells after being contacted with the M1 peptide and/or IL-2 is at least 80% of the CD8⁺ cells. In some embodiments, the percentage of Vβ17⁺CD8⁺ T cells among CD8⁺ T cells after being contacted with the M1 peptide and/or IL-2 is at least 85% of the CD8⁺ cells. In some embodiments, the percentage of Vβ17⁺CD8⁺ T cells among CD8⁺ T cells after being contacted with the M1 peptide and/or IL-2 is at least 90% of the CD8⁺ cells. In some embodiments, the percentage of Vβ17⁺CD8⁺ T cells among CD8⁺ T cells is about 6% to 99% of the CD8⁺ cells after being contacted with the M1 peptide. In some embodiments, the percentage of Vβ17⁺CD8⁺ T cells among CD8⁺ T cells is about 6% to 99% of the CD8⁺ cells after being contacted with IL-2. In some embodiments, the percentage of Vβ17⁺CD8⁺ T cells among CD8⁺ T cells is about 6% to 99% of the CD8⁺ cells after being contacted with the M1 peptide and IL-2.

In some embodiments, the percentage of Vβ17⁺CD8⁺ T cells among CD8⁺ T cells after being contacted with the M1 peptide and/or IL-2 is 6% to 99% of the CD8⁺ cells. In some embodiments, the percentage of Vβ17⁺CD8⁺ T cells among CD8⁺ T cells after being contacted with the M1 peptide and/or IL-2 is 10% to 99% of the CD8⁺ cells. In some embodiments, the percentage of Vβ17⁺CD8⁺ T cells among CD8⁺ T cells after being contacted with the M1 peptide and/or IL-2 is 15% to 99% of the CD8⁺ cells. In some embodiments, the percentage of Vβ17⁺CD8⁺ T cells among CD8⁺ T cells after being contacted with the M1 peptide and/or IL-2 is 20% to 99% of the CD8⁺ cells. In some embodiments, the percentage of Vβ17⁺CD8⁺ T cells among CD8⁺ T cells after being contacted with the M1 peptide and/or IL-2 is 25% to 99% of the CD8⁺ cells. In some embodiments, the percentage of Vβ17⁺CD8⁺ T cells among CD8⁺ T cells after being contacted with the M1 peptide and/or IL-2 is 30% to 99% of the CD8⁺ cells. In some embodiments, the percentage of Vβ17⁺CD8⁺ T cells among CD8⁺ T cells after being contacted with the M1 peptide and/or IL-2 is 35% to 99% of the CD8+ cells. In some embodiments, the percentage of Vβ17⁺CD8⁺ T cells among CD8⁺ T cells after being contacted with the M1 peptide and/or IL-2 is 40% to 99% of the CD8+ cells. In some embodiments, the percentage of Vβ17⁺CD8⁺ T cells among CD8⁺ T cells after being contacted with the M1 peptide and/or IL-2 is 45% to 99% of the CD8+ cells. In some embodiments, the percentage of Vβ17⁺CD8⁺ T cells among CD8⁺ T cells after being contacted with the M1 peptide and/or IL-2 is 50% to 99% of the CD8+ cells. In some embodiments, the percentage of Vβ17⁺CD8⁺ T cells among CD8⁺ T cells after being contacted with the M1 peptide and/or IL-2 is 55% to 99% of the CD8⁺ cells. In some embodiments, the percentage of Vβ17⁺CD8⁺ T cells among CD8⁺ T cells after being contacted with the M1 peptide and/or IL-2 is 60% to 99% of the CD8+ cells. In some embodiments, the percentage of Vβ17⁺CD8⁺ T cells among CD8⁺ T cells after being contacted with the M1 peptide and/or IL-2 is 65% to 99% of the CD8+ cells. In some embodiments, the percentage of Vβ17⁺CD8⁺ T cells among CD8⁺ T cells after being contacted with the M1 peptide and/or IL-2 is 70% to 99% of the CD8+ cells. In some embodiments, the percentage of Vβ17⁺CD8⁺ T cells among CD8⁺ T cells after being contacted with the M1 peptide and/or IL-2 is 75% to 99% of the CD8+ cells. In some embodiments, the percentage of Vβ17⁺CD8⁺ T cells among CD8⁺ T cells after being contacted with the M1 peptide and/or IL-2 is 80% to 99% of the CD8+ cells. In some embodiments, the percentage of Vβ17⁺CD8⁺ T cells among CD8⁺ T cells after being contacted with the M1 peptide and/or IL-2 is 85% to 99% of the CD8+ cells. In some embodiments, the percentage of Vβ17⁺CD8⁺ T cells among CD8⁺ T cells after being contacted with the M1 peptide and/or IL-2 is 90% to 99% of the CD8+ cells. In some embodiments, the percentage of Vβ17⁺CD8⁺ T cells among CD8⁺ T cells is 6% to 99% of the CD8⁺ cells after being contacted with the M1 peptide. In some embodiments, the percentage of Vβ17⁺CD8⁺ T cells among CD8⁺ T cells is 6% to 99% of the CD8⁺ cells after being contacted with IL-2. In some embodiments, the percentage of Vβ17⁺CD8⁺ T cells among CD8⁺ T cells is 6% to 99% of the CD8⁺ cells after being contacted with the M1 peptide and IL-2.

### 5.2.3. Enrichment of CD8 Positive Cells

In some embodiments, the methods or processes provided here comprises isolating or enriching Vβ17⁺CD8⁺ T cells after M1 peptide stimulation.

In a specific embodiments, the method comprises isolating CD8+ cells first and then isolating Vβ17 positive cells. In a specific embodiments, the method comprises isolating Vβ17 positive cells first and then isolating CD8+ cells. In another specific embodiments, the method comprises isolating CD8+ cells and Vβ17 positive cells simultaneously.

CD8 is a transmembrane glycoprotein expressed on the surface of cytotoxic T-cells. CD8 forms a homo- or heterodimer comprised of either CD8 alpha and/or CD8 beta chains. CD8 interacts with class I MHC receptors during antigen-specific activation, and participates in T-cell receptor-mediated activation.

The method of isolating and enriching CD8+ cells are known to those of skill in the art. In some embodiments, the method of enriching CD8+ cells comprises positive or negative selection.

In some embodiments, the method of enriching CD8+ cells comprises using magnetic beads that are directly coated with anti-CD8 antibody or using anti-CD8 antibodies to coat cells and then the beads with a secondary reagent to bind antibodies. In some embodiments, the method of enriching CD8+ cells comprises using magnetic microparticles that are directly coated with anti-CD8 antibody or using anti-CD8 antibodies to coat cells and then the magnetic microparticles with a secondary reagent to bind antibodies. In some embodiments, the method of enriching CD8+ cells comprises using magnetic nanoparticle that are directly coated with anti-CD8 antibody or using anti-CD8 antibodies to coat cells and then the magnetic nanoparticle with a secondary reagent to bind antibodies. In some embodiments, the method of enriching CD8+ cells comprises density gradient centrifugation, for example but not limiting to using albumin, dextran, Ficoll, metrizamid, Percoll and the like, to remove undesired cells.

In other embodiments, the method of enriching CD8+ cells comprises a negative selection by incubating the cell mixture with reagents that bind to undesired cells. In some embodiments, the method of enriching CD8+ cells comprises a positive selection by selecting out cells with surface expression of CD8. In some specific embodiments, the method of enriching CD8+ cells comprises FACS sorting of CD8+ cells. In some other specific embodiments, the method of enriching CD8+ cells comprises using an anti-CD8 antibody which targets any of CD8 chains. In some specific embodiments, the method of enriching CD8+ cells comprises an affinity column immobilized with a binding agent to CD8. In some embodiments, the method of enriching CD8+ cells comprises a combination of two or more of the above methods.

In some more specific embodiments, the method for enriching or isolating CD8+ cells is as described in Section 7 below.

### 5.2.4. Enrichment of Vβ17 Positive Cells

In some embodiments, the method, provided herein, of enriching Vβ17 positive cells comprises selecting out cells with surface expression of Vβ17. In some other specific embodiments, the method of enriching Vβ17 positive cells comprises using an anti-Vβ17 antibody. In some embodiments, the method of enriching Vβ17 positive cells comprises using magnetic beads that are directly coated with anti-Vβ17 antibody or using anti-Vβ17 antibodies to coat cells and then the beads with a secondary reagent to bind antibodies. In some embodiments, the method of enriching Vβ17 positive cells comprises using magnetic microparticles that are directly coated with anti-Vβ17 antibody or using anti-Vβ17 antibodies to coat cells and then the magnetic microparticles with a secondary reagent to bind antibodies. In some embodiments, the method of enriching Vβ17 positive cells comprises using magnetic nanoparticle that are directly coated with anti-Vβ17 antibody or using anti-Vβ17 antibodies to coat cells and then the magnetic nanoparticle with a secondary reagent to bind antibodies. In some specific embodiments, the method of enriching Vβ17 positive cells comprises FACS sorting of Vβ17 positive cells. In some specific embodiments, the method of enriching Vβ17 positive cells comprises an affinity column immobilized with a binding agent to Vβ17. In some embodiments, the method of enriching Vβ17 positive cells comprises a combination of two or more of the above methods.

In some more specific embodiments, the method for enriching or isolating Vβ17 positive cells is as described in Section 7 below.

5.2.5. Activation of the Enriched Vβ17⁺CD8⁺ T Cells

The method provided herein further comprises a step of activation and/or expansion of Vβ17⁺CD8⁺ T cells.

In some embodiments, the step of activation and/or expansion of Vβ17⁺CD8⁺ T cells comprises adding cytokines to the cells, and the cytokines include but are not limited to lectin, hepatic growth factor, prostaglandin, fibroblast growth factor, prolactin, placental lactogen, OB protein, tumor necrosis factor-α, tumor necrosis factor-β, mullerian-inhibiting substance, mouse gonadotropin-associated peptide, inhibin, activin, vascular endothelial growth factor, integrin, thrombopoietin (TPO), a nerve growth factor (NGF), platelet-growth factor, TGF-α, TGF-β, insulin-like growth factor-I, insulin-like growth factor-II, erythropoietin (EPO), an osteoinductive factor, interferon-α, interferon-β, interferon-λ, macrophage-CSF (M-CSF), granulocyte-macrophage-CSF (GM-CSF), granulocyte-CSF (G-CSF), interleukin-1 (IL-1), IL-1a, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, LIF, kit-ligand, FLT-3, angiostatin, thrombospondin, endostatin, tumor necrosis factor and LT (lymphotoxin). In some embodiments, the step of activation and/or expansion of Vβ17⁺CD8⁺ T cells comprises co-culturing the cells with feeder cells. In some embodiments, the step of activation and/or expansion of Vβ17⁺CD8⁺ T cells comprises adding an agent that inhibit immunosuppressive signals. In specific embodiments, the step of activation and/or expansion of Vβ17⁺CD8⁺ T cells comprises immune checkpoint inhibitors. In specific embodiments, the step of activation and/or expansion of Vβ17⁺CD8⁺ T cells comprises anti-PD-1 antibody or antigen-binding fragment thereof. In a specific embodiment, the step of activation and/or expansion of Vβ17⁺CD8⁺ T cells comprises anti-CD3/CD28 beads.

### 5.3. Vβ17⁺CD8⁺ T Cells

In another aspect, provided herein is a T cell, wherein the T cell is a Vβ17⁺CD8⁺ T cell, and wherein the T cell express a cell surface receptor capable of binding the M1 peptide comprising the amino acid sequence of SEQ ID NO:1.

In another aspect, provided herein is an isolated population of Vβ17⁺CD8⁺ T cells produced by the method provided herein. In some embodiments, the Vβ17⁺CD8⁺ T cells comprises a cell surface receptor (e.g., TCR) capable of binding M1 peptide comprising the amino acid sequence of SEQ ID NO:1.

In yet another aspect, provided herein is an isolated population of cells, wherein the percentage of Vβ17⁺CD8⁺ T cells in the isolated population of the cells is more than 6%, 10%, 15%, 20%, 25%, 30%, 35%, 40%, 45%, 50%, 55%, 60%, 65%, 70%, 75%, 80%, 85%, or 90% of the isolated population of the cells. In some embodiments, provided herein is an isolated population of cells, wherein the percentage of Vβ17⁺CD8⁺ T cells in the isolated population of the cells is more than 10% of the isolated population of the cells. In some embodiments, provided herein is an isolated population of cells, wherein the percentage of Vβ17⁺CD8⁺ T cells in the isolated population of the cells is more than 15% of the isolated population of the cells. In some embodiments, provided herein is an isolated population of cells, wherein the percentage of Vβ17⁺CD8⁺ T cells in the isolated population of the cells is more than 20% of the isolated population of the cells. In some embodiments, provided herein is an isolated population of cells, wherein the percentage of Vβ17⁺CD8⁺ T cells in the isolated population of the cells is more than 25% of the isolated population of the cells. In some embodiments, provided herein is an isolated population of cells, wherein the percentage of Vβ17⁺CD8⁺ T cells in the isolated population of the cells is more than 30% of the isolated population of the cells. In some embodiments, provided herein is an isolated population of cells, wherein the percentage of Vβ17⁺CD8⁺ T cells in the isolated population of the cells is more than 35% of the isolated population of the cells. In some embodiments, provided herein is an isolated population of cells, wherein the percentage of Vβ17⁺CD8⁺ T cells in the isolated population of the cells is more than 40% of the isolated population of the cells. In some embodiments, provided herein is an isolated population of cells, wherein the percentage of Vβ17⁺CD8⁺ T cells in the isolated population of the cells is more than 45% of the isolated population of the cells. In some embodiments, provided herein is an isolated population of cells, wherein the percentage of Vβ17⁺CD8⁺ T cells in the isolated population of the cells is more than 50% of the isolated population of the cells. In some embodiments, provided herein is an isolated population of cells, wherein the percentage of Vβ17⁺CD8⁺ T cells in the isolated population of the cells is more than 55% of the isolated population of the cells. In some embodiments, provided herein is an isolated population of cells, wherein the percentage of Vβ17⁺CD8⁺ T cells in the isolated population of the cells is more than 60% of the isolated population of the cells. In some embodiments, provided herein is an isolated population of cells, wherein the percentage of Vβ17⁺CD8⁺ T cells in the isolated population of the cells is more than 65% of the isolated population of the cells. In some embodiments, provided herein is an isolated population of cells, wherein the percentage of Vβ17⁺CD8⁺ T cells in the isolated population of the cells is more than 70% of the isolated population of the cells. In some embodiments, provided herein is an isolated population of cells, wherein the percentage of Vβ17⁺CD8⁺ T cells in the isolated population of the cells is more than 75% of the isolated population of the cells. In some embodiments, provided herein is an isolated population of cells, wherein the percentage of Vβ17⁺CD8⁺ T cells in the isolated population of the cells is more than 80% of the isolated population of the cells. In some embodiments, provided herein is an isolated population of cells, wherein the percentage of Vβ17⁺CD8⁺ T cells in the isolated population of the cells is more than 85% of the isolated population of the cells. In some embodiments, provided herein is an isolated population of cells, wherein the percentage of Vβ17⁺CD8⁺ T cells in the isolated population of the cells is more than 90% of the isolated population of the cells. In some embodiments, provided herein is an isolated population of cells, wherein the percentage of Vβ17⁺CD8⁺ T cells in the isolated population of the cells is more than 95% of the isolated population of the cells.

In some embodiments, provided herein is an isolated population of cells, wherein the percentage of Vβ17⁺CD8⁺ T cells in the isolated population of the cells is 6% to 99% of the isolated population of the cells. In some embodiments, provided herein is an isolated population of cells, wherein the percentage of Vβ17⁺CD8⁺ T cells in the isolated population of the cells is 10% to 99% of the isolated population of the cells. In some embodiments, provided herein is an isolated population of cells, wherein the percentage of Vβ17⁺CD8⁺ T cells in the isolated population of the cells is 15% to 99% of the isolated population of the cells. In some embodiments, provided herein is an isolated population of cells, wherein the percentage of Vβ17⁺CD8⁺ T cells in the isolated population of the cells is 20% to 99% of the isolated population of the cells. In some embodiments, provided herein is an isolated population of cells, wherein the percentage of Vβ17⁺CD8⁺ T cells in the isolated population of the cells is 25% to 99% of the isolated population of the cells. In some embodiments, provided herein is an isolated population of cells, wherein the percentage of Vβ17⁺CD8⁺ T cells in the isolated population of the cells is 30% to 99% of the isolated population of the cells. In some embodiments, provided herein is an isolated population of cells, wherein the percentage of Vβ17⁺CD8⁺ T cells in the isolated population of the cells is 35% to 99% of the isolated population of the cells. In some embodiments, provided herein is an isolated population of cells, wherein the percentage of Vβ17⁺CD8⁺ T cells in the isolated population of the cells is 40% to 99% of the isolated population of the cells. In some embodiments, provided herein is an isolated population of cells, wherein the percentage of Vβ17⁺CD8⁺ T cells in the isolated population of the cells is 45% to 99% of the isolated population of the cells. In some embodiments, provided herein is an isolated population of cells, wherein the percentage of Vβ17⁺CD8⁺ T cells in the isolated population of the cells is 50% to 99% of the isolated population of the cells. In some embodiments, provided herein is an isolated population of cells, wherein the percentage of Vβ17⁺CD8⁺ T cells in the isolated population of the cells is 55% to 99% of the isolated population of the cells. In some embodiments, provided herein is an isolated population of cells, wherein the percentage of Vβ17⁺CD8⁺ T cells in the isolated population of the cells is 60% to 99% of the isolated population of the cells. In some embodiments, provided herein is an isolated population of cells, wherein the percentage of Vβ17⁺CD8⁺ T cells in the isolated population of the cells is 65% to 99% of the isolated population of the cells. In some embodiments, provided herein is an isolated population of cells, wherein the percentage of Vβ17⁺CD8⁺ T cells in the isolated population of the cells is 70% to 99% of the isolated population of the cells. In some embodiments, provided herein is an isolated population of cells, wherein the percentage of Vβ17⁺CD8⁺ T cells in the isolated population of the cells is 75% to 99% of the isolated population of the cells. In some embodiments, provided herein is an isolated population of cells, wherein the percentage of Vβ17⁺CD8⁺ T cells in the isolated population of the cells is 80% to 99% of the isolated population of the cells. In some embodiments, provided herein is an isolated population of cells, wherein the percentage of Vβ17⁺CD8⁺ T cells in the isolated population of the cells is 85% to 99% of the isolated population of the cells. In some embodiments, provided herein is an isolated population of cells, wherein the percentage of Vβ17⁺CD8⁺ T cells in the isolated population of the cells is 90% to 99% of the isolated population of the cells.

In some embodiments, at least part of the Vβ17⁺CD8⁺ T cells express a cell surface receptor (e.g., TCR) capable of binding to the M1 peptide.

In yet another aspect, provided herein are methods for using the Vβ17⁺CD8⁺ T cells provided herein, for example, herein in combination with T cell engagers as described in more detail below in Section 5.4 to Section 5.7 below.

### 5.4. T Cell Engagers

In another aspect, provided herein are methods for using the Vβ17⁺CD8⁺ T cells provided herein in combination with T cell engagers so that the Vβ17⁺CD8⁺ T cells are directed to target cells, thereby for example inhibiting the growth or proliferation of target cells or eliminating target cells.

In some embodiments, each of the T cell engagers is a multi-specific antibody. In some embodiments, the multispecific antibodies are trispecific. In some embodiments, the multispecific antibodies are bispecific. In some embodiments, the antibody is a humanized antibody. In certain embodiments, the antibody is an IgG antibody. In other embodiments, the IgG antibody is an IgG1, IgG2, IgG3, or IgG4 antibody. In some embodiments, the multispecific antibody comprises a first binding domain that binds to an antigen expressed on a Vβ17⁺CD8⁺ T cell and a second binding domain that binds to an antigen expressed on an unhealthy cell.

Antigens expressed on Vβ17⁺CD8⁺ T cells are well known in the art. In some embodiments, the antigen expressed on the Vβ17⁺CD8⁺ T cell is T Cell Receptor Beta Variable 19. In some embodiments, the antigen expressed on the Vβ17⁺CD8⁺ T cell is Vβ17.

In an embodiment of the bispecific antibodies provided herein, the first epitope is located on TRBV19 and the second epitope is located on the surface of a cancer cell. In some embodiments, the second epitope is located on a cancer cell antigen. In an embodiment of the bispecific antibodies provided herein, the first epitope is located on TRBV19 and the second epitope is located on a tumor. In an embodiment of the bispecific antibodies provided herein, the first epitope is located on TRBV19 and the second epitope is located on a tumor-specific antigen. In an embodiment of the bispecific antibodies provided herein, the first epitope is located on TRBV19 and the second epitope is located on a tumor associated antigen. In an embodiment of the bispecific antibodies provided herein, the first epitope is located on TRBV19 and the second epitope is located on a neoantigen.

In an embodiment of the bispecific antibodies provided herein, the first epitope is located on Vβ17 and the second epitope is located on the surface of a cancer cell. In some embodiments, the second epitope is located on a cancer cell antigen. In an embodiment of the bispecific antibodies provided herein, the first epitope is located on Vβ17 and the second epitope is located on a tumor. In an embodiment of the bispecific antibodies provided herein, the first epitope is located on Vβ17 and the second epitope is located on a tumor-specific antigen. In an embodiment of the bispecific antibodies provided herein, the first epitope is located on Vβ17 and the second epitope is located on a tumor associated antigen. In an embodiment of the bispecific antibodies provided herein, the first epitope is located on Vβ17 and the second epitope is located on a neoantigen.

In some embodiments, the unhealthy cell is from a subject has an autoimmune and inflammatory disease. In some embodiments, the unhealthy cell is from a subject has a neurological disease. In some embodiments, the unhealthy cell is a cancer cell. In certain embodiments, the cancer cell is a blood cancer cell or a solid tumor cancer cell. In some embodiments, the cancer cell is a cell of an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma, neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer endometrial cancer, vaginal cancer, or vulvar cancer. In some embodiments, the cancer is an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma, neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer endometrial cancer, vaginal cancer, or vulvar cancer. In some embodiments, the cancer is a adrenal cancer. In some embodiments, the cancer is an anal cancer. In some embodiments, the cancer is an appendix cancer. In some embodiments, the cancer is a bile duct cancer. In some embodiments, the cancer is a bladder cancer. In some embodiments, the cancer is a bone cancer. In some embodiments, the cancer is a brain cancer. In some embodiments, the cancer is a breast cancer. In some embodiments, the cancer is a cervical cancer. In some embodiments, the cancer is a colorectal cancer. In some embodiments, the cancer is an esophageal cancer. In some embodiments, the cancer is a gallbladder cancer. In some embodiments, the cancer is a gestational trophoblastic. In some embodiments, the cancer is a head and neck cancer. In some embodiments, the cancer is a Hodgkin lymphoma. In some embodiments, the cancer is an intestinal cancer. In some embodiments, the cancer is a kidney cancer. In some embodiments, the cancer is a leukemia. In some embodiments, the cancer is a liver cancer. In some embodiments, the cancer is a lung cancer. In some embodiments, the cancer is a melanoma. In some embodiments, the cancer is a mesothelioma. In some embodiments, the cancer is a multiple myeloma. In some embodiments, the cancer is a neuroendocrine tumor. In some embodiments, the cancer is a non-Hodgkin lymphoma. In some embodiments, the cancer is an oral cancer. In some embodiments, the cancer is an ovarian cancer. In some embodiments, the cancer is a pancreatic cancer. In some embodiments, the cancer is a prostate cancer. In some embodiments, the cancer is a sinus cancer. In some embodiments, the cancer is a skin cancer. In some embodiments, the cancer is a soft tissue sarcoma spinal cancer. In some embodiments, the cancer is a stomach cancer. In some embodiments, the cancer is a testicular cancer. In some embodiments, the cancer is a throat cancer. In some embodiments, the cancer is a thyroid cancer. In some embodiments, the cancer is a uterine cancer endometrial cancer. In some embodiments, the cancer is a vaginal cancer. In some embodiments, the cancer is a vulvar cancer.

In some embodiments, the adrenal cancer is an adrenocortical carcinoma (ACC), adrenal cortex cancer, pheochromocytoma, or neuroblastoma. In some embodiments, the anal cancer is a squamous cell carcinoma, cloacogenic carcinoma, adenocarcinoma, basal cell carcinoma, or melanoma. In some embodiments, the appendix cancer is a neuroendocrine tumor (NET), mucinous adenocarcinoma, goblet cell carcinoid, intestinal-type adenocarcinoma, or signet-ring cell adenocarcinoma. In some embodiments, the bile duct cancer is an extrahepatic bile duct cancer, adenocarcinomas, hilar bile duct cancer, perihilar bile duct cancer, distal bile duct cancer, or intrahepatic bile duct cancer. In some embodiments, the bladder cancer is transitional cell carcinoma (TCC), papillary carcinoma, flat carcinoma, squamous cell carcinoma, adenocarcinoma, small-cell carcinoma, or sarcoma. In some embodiments, the bone cancer is a primary bone cancer, sarcoma, osteosarcoma, chondrosarcoma, sarcoma, fibrosarcoma, malignant fibrous histiocytoma, giant cell tumor of bone, chordoma, or metastatic bone cancer. In some embodiments, the brain cancer is an astrocytoma, brain stem glioma, glioblastoma, meningioma, ependymoma, oligodendroglioma, mixed glioma, pituitary carcinoma, pituitary adenoma, craniopharyngioma, germ cell tumor, pineal region tumor, medulloblastoma, or primary CNS lymphoma. In some embodiments, the breast cancer is a breast adenocarcinoma, invasive breast cancer, noninvasive breast cancer, breast sarcoma, metaplastic carcinoma, adenocystic carcinoma, phyllodes tumor, angiosarcoma, HER2-positive breast cancer, triple-negative breast cancer, or inflammatory breast cancer. In some embodiments, the cervical cancer is a squamous cell carcinoma, or adenocarcinoma. In some embodiments, the colorectal cancer is a colorectal adenocarcinoma, primary colorectal lymphoma, gastrointestinal stromal tumor, leiomyosarcoma, carcinoid tumor, mucinous adenocarcinoma, signet ring cell adenocarcinoma, gastrointestinal carcinoid tumor, or melanoma. In some embodiments, the esophageal cancer is an adenocarcinoma or squamous cell carcinoma. In some embodiments, the gall bladder cancer is an adenocarcinoma, papillary adenocarcinoma, adenosquamous carcinoma, squamous cell carcinoma, small cell carcinoma, or sarcoma. In some embodiments, the gestational trophoblastic disease (GTD) is a hydatidiform mole, gestational trophoblastic neoplasia (GTN), choriocarcinoma, placental-site trophoblastic tumor (PSTT), or epithelioid trophoblastic tumor (ETT). In some embodiments, the head and neck cancer is a laryngeal cancer, nasopharyngeal cancer, hypopharyngeal cancer, nasal cavity cancer, paranasal sinus cancer, salivary gland cancer, oral cancer, oropharyngeal cancer, or tonsil cancer. In some embodiments, the Hodgkin lymphoma is a classical Hodgkin lymphoma, nodular sclerosis, mixed cellularity, lymphocyte-rich, lymphocyte-depleted, or nodular lymphocyte-predominant Hodgkin lymphoma (NLPHL). In some embodiments, the intestinal cancer is a small intestine cancer, small bowel cancer, adenocarcinoma, sarcoma, gastrointestinal stromal tumors, carcinoid tumors, or lymphoma. In some embodiments, the kidney cancer is a renal cell carcinoma (RCC), clear cell RCC, papillary RCC, chromophobe RCC, collecting duct RCC, unclassified RCC, transitional cell carcinoma, urothelial cancer, renal pelvis carcinoma, or renal sarcoma. In some embodiments, the leukemia is an acute lymphocytic leukemia (ALL), acute myeloid leukemia (AML), chronic lymphocytic leukemia (CLL), chronic myeloid leukemia (CML), hairy cell leukemia (HCL), or a myelodysplastic syndrome (MDS). In a specific embodiment, the leukemia is AML. In some embodiments, the liver cancer is a hepatocellular carcinoma (HCC), fibrolamellar HCC, cholangiocarcinoma, angiosarcoma, or liver metastasis. In some embodiments, the lung cancer is a small cell lung cancer, small cell carcinoma, combined small cell carcinoma, non-small cell lung cancer, lung adenocarcinoma, squamous cell lung cancer, large-cell undifferentiated carcinoma, pulmonary nodule, metastatic lung cancer, adenosquamous carcinoma, large cell neuroendocrine carcinoma, salivary gland-type lung carcinoma, lung carcinoid, mesothelioma, sarcomatoid carcinoma of the lung, or malignant granular cell lung tumor. In some embodiments, the melanoma is a superficial spreading melanoma, nodular melanoma, acral-lentiginous melanoma, lentigo maligna melanoma, amelanotic melanoma, desmoplastic melanoma, ocular melanoma, or metastatic melanoma. In some embodiments, the mesothelioma is a pleural mesothelioma, peritoneal mesothelioma, pericardial mesothelioma, or testicular mesothelioma. In some embodiments, the multiple myeloma is an active myeloma or smoldering myeloma. In some embodiments, the neuroendocrine tumor, is a gastrointestinal neuroendocrine tumor, pancreatic neuroendocrine tumor, or lung neuroendocrine tumor. In some embodiments, the non-Hodgkin's lymphoma is an anaplastic large-cell lymphoma, lymphoblastic lymphoma, peripheral T cell lymphoma, follicular lymphoma, cutaneous T cell lymphoma, lymphoplasmacytic lymphoma, marginal zone B-cell lymphoma, MALT lymphoma, small-cell lymphocytic lymphoma, Burkitt lymphoma, chronic lymphocytic leukemia (CLL), small lymphocytic lymphoma (SLL), precursor T-lymphoblastic leukemia/lymphoma, acute lymphocytic leukemia (ALL), adult T cell lymphoma/leukemia (ATLL), hairy cell leukemia, B-cell lymphomas, diffuse large B-cell lymphoma (DLBCL), primary mediastinal B-cell lymphoma, primary central nervous system (CNS) lymphoma, mantle cell lymphoma (MCL), marginal zone lymphomas, mucosa-associated lymphoid tissue (MALT) lymphoma, nodal marginal zone B-cell lymphoma, splenic marginal zone B-cell lymphoma, lymphoplasmacytic lymphoma, B-cell non-Hodgkin lymphoma, T cell non-Hodgkin lymphoma, natural killer cell lymphoma, cutaneous T cell lymphoma, Alibert-Bazin syndrome, Sezary syndrome, primary cutaneous anaplastic large-cell lymphoma, peripheral T cell lymphoma, angioimmunoblastic T cell lymphoma (AITL), anaplastic large-cell lymphoma (ALCL), systemic ALCL, enteropathy-type T cell lymphoma (EATL), or hepatosplenic gamma/delta T cell lymphoma. In some embodiments, the oral cancer is a squamous cell carcinoma, verrucous carcinoma, minor salivary gland carcinomas, lymphoma, benign oral cavity tumor, eosinophilic granuloma, fibroma, granular cell tumor, karatoacanthoma, leiomyoma, osteochondroma, lipoma, schwannoma, neurofibroma, papilloma, condyloma acuminatum, verruciform xanthoma, pyogenic granuloma, rhabdomyoma, odontogenic tumors, leukoplakia, erythroplakia, squamous cell lip cancer, basal cell lip cancer, mouth cancer, gum cancer, or tongue cancer. In some embodiments, the ovarian cancer is an ovarian epithelial cancer, mucinous epithelial ovarian cancer, endometrioid epithelial ovarian cancer, clear cell epithelial ovarian cancer, undifferentiated epithelial ovarian cancer, ovarian low malignant potential tumors, primary peritoneal carcinoma, fallopian tube cancer, germ cell tumors, teratoma, dysgerminoma ovarian germ cell cancer, endodermal sinus tumor, sex cord-stromal tumors, sex cord-gonadal stromal tumor, ovarian stromal tumor, granulosa cell tumor, granulosa-theca tumor, Sertoli-Leydig tumor, ovarian sarcoma, ovarian carcinosarcoma, ovarian adenosarcoma, ovarian leiomyosarcoma, ovarian fibrosarcoma, Krukenberg tumor, or ovarian cyst. In some embodiments, the pancreatic cancer is a pancreatic exocrine gland cancer, pancreatic endocrine gland cancer, or pancreatic adenocarcinoma, islet cell tumor, or neuroendocrine tumor. In some embodiments, the prostate cancer is a prostate adenocarcinoma, prostate sarcoma, transitional cell carcinoma, small cell carcinoma, or neuroendocrine tumor. In some embodiments, the sinus cancer is a squamous cell carcinoma, mucosa cell carcinoma, adenoid cystic cell carcinoma, acinic cell carcinoma, sinonasal undifferentiated carcinoma, nasal cavity cancer, paranasal sinus cancer, maxillary sinus cancer, ethmoid sinus cancer, or nasopharynx cancer. In some embodiments, the skin cancer is a basal cell carcinoma, squamous cell carcinoma, melanoma, Merkel cell carcinoma, Kaposi sarcoma (KS), actinic keratosis, skin lymphoma, or keratoacanthoma. In some embodiments, the soft tissue cancer is an angiosarcoma, dermatofibrosarcoma, epithelioid sarcoma, Ewing's sarcoma, fibrosarcoma, gastrointestinal stromal tumors (GISTs), Kaposi sarcoma, leiomyosarcoma, liposarcoma, dedifferentiated liposarcoma (DL), myxoid/round cell liposarcoma (MRCL), well-differentiated liposarcoma (WDL), malignant fibrous histiocytoma, neurofibrosarcoma, rhabdomyosarcoma (RMS), or synovial sarcoma. In some embodiments, the spinal cancer is a spinal metastatic tumor. In some embodiments, the stomach cancer is a stomach adenocarcinoma, stomach lymphoma, gastrointestinal stromal tumors, carcinoid tumor, gastric carcinoid tumors, Type I ECL-cell carcinoid, Type II ECL-cell carcinoid, or Type III ECL-cell carcinoid. In some embodiments, the testicular cancer is a seminoma, non-seminoma, embryonal carcinoma, yolk sac carcinoma, choriocarcinoma, teratoma, gonadal stromal tumor, leydig cell tumor, or sertoli cell tumor. In some embodiments, the throat cancer is a squamous cell carcinoma, adenocarcinoma, sarcoma, laryngeal cancer, pharyngeal cancer, nasopharynx cancer, oropharynx cancer, hypopharynx cancer, laryngeal cancer, laryngeal squamous cell carcinoma, laryngeal adenocarcinoma, lymphoepithelioma, spindle cell carcinoma, verrucous cancer, undifferentiated carcinoma, or lymph node cancer. In some embodiments, the thyroid cancer is a papillary carcinoma, follicular carcinoma, Hürthle cell carcinoma, medullary thyroid carcinoma, or anaplastic carcinoma. In some embodiments, the uterine cancer is an endometrial cancer, endometrial adenocarcinoma, endometroid carcinoma, serous adenocarcinoma, adenosquamous carcinoma, uterine carcinosarcoma, uterine sarcoma, uterine leiomyosarcoma, endometrial stromal sarcoma, or undifferentiated sarcoma. In some embodiments, the vaginal cancer is a squamous cell carcinoma, adenocarcinoma, melanoma, or sarcoma. In some embodiments, the vulvar cancer is a squamous cell carcinoma or adenocarcinoma.

In some embodiments, the antigen expressed on the unhealthy cell is a tumor antigen. In some embodiments, the tumor antigen is a tumor-specific antigen (TSA) or a tumor-associated antigen (TAA). A TSA is unique to tumor cells and does not occur on other cells in the body. A TAA is not unique to a tumor cell, and instead is also expressed on a normal cell under conditions that induce a state of immunologic tolerance to the antigen. The expression of the antigen on the tumor may occur under conditions that enable the immune system to respond to the antigen. TAAs may be antigens that are expressed on normal cells during fetal development, when the immune system is immature and unable to respond, or they may be antigens that are normally present at extremely low levels on normal cells but are expressed at much higher levels on tumor cells.

Non-limiting examples of TSA or TAA antigens include: differentiation antigens such as MART-1/MelanA (MART-I), gp 100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. In some embodiments, the TAA is CD123. In some embodiments, the TAA is PSMA. In some embodiments, the TAA is ILIRAP. In some embodiments, the TAA is B7H3.

Other large, protein-based antigens include TSP-180, MAGE-4, MAGE-5, MAGE-6, RAGE, NY-ESO, p185erbB2, p180erbB-3, c-met, nm-23HI, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS 1, SDCCAG16, TA-90\Mac-2 binding protein\cyclophilin C-associated protein, TAAL6, TAG72, TLP, and TPS.

In some embodiments, the bispecific antibody provided herein comprises an TRBV19 binding arm. In some embodiments, the bispecific antibodies provided herein have high-affinity binding to TRBV19. In some embodiments, the bispecific antibodies provided herein have high specificity to TRBV19. In some embodiments, the bispecific antibodies provided herein have high-affinity binding to a second target antigen. In some embodiments, the bispecific antibodies provided herein have high-affinity binding to a second target antigen. In some embodiments, the bispecific antibodies provided herein have high specificity to a second target antigen. In some embodiments, the second target antigen is an antigen expressed on a tumor cell. In some embodiments, the bispecific antibodies provided herein have high specificity to CD123. In some, embodiments, the bispecific antibodies provided herein have high specificity to PSMA. In some embodiments, the bispecific antibodies provided herein have high specificity to ILIRAP. In some, embodiments, the bispecific antibodies provided herein have high specificity to B7H3. In some embodiments, the bispecific antibodies provided herein have the ability to treat or prevent a disease or disorder when administered alone. In some embodiments, the bispecific antibodies provided herein have the ability to treat or prevent a disease or disorder when administered in combination with other therapies. In some embodiments, the disease or disorder is a cancer. In some embodiments, the disease or disorder is a blood cancer. In some embodiments, the disease or disorder is a solid tumor cancer.

In certain embodiments, provided herein is an anti-TRBV19 bispecific antibody comprising a binding domain that binds to TRBV19 having a VH region, VL region, VH CDR1, VH CDR2, VH CDR3, VL CDR1, VL CDR2, and/or VL CDR3 of any one of the antibodies described herein. In some embodiments, provided herein is an anti-TRBV19 bispecific antibody comprising a binding domain that binds to TRBV19 having a VH region of any one of the antibodies described herein. In some embodiments, provided herein is an anti-TRBV19 bispecific antibody comprising a binding domain that binds to TRBV19 having a VL region of any one of the antibodies described herein. In some embodiments, provided herein is an anti-TRBV19 bispecific antibody comprising a binding domain that binds to TRBV19 having a VH region of any one of the antibodies described herein, and a VL region of any one of the antibodies described herein. In some embodiments, provided herein is an anti-TRBV19 bispecific antibody comprising a binding domain that binds to TRBV19 having a VH CDR1, VH CDR2, and VH CDR3 of any one of the antibodies described. In some embodiments, provided herein is an anti-TRBV19 bispecific antibody comprising a binding domain that binds to TRBV19 having a VL CDR1, VL CDR2, and VL CDR3 of any one of the antibodies described herein. In some embodiments, provided herein is an anti-TRBV19 bispecific antibody comprising a binding domain that binds to TRBV19 having a VH CDR1, VH CDR2, and VH CDR3 of any one of the antibodies described herein; and a VL CDR1, VL CDR2, and VL CDR3 of any one of the antibodies described herein.

In some embodiments, the anti-TRBV19 bispecific antibody further comprises a second binding domain that binds to CD123 having a VH region, VL region, VH CDR1, VH CDR2, VH CDR3, VL CDR1, VL CDR2, and/or VL CDR3 of an anti-CD123 antibody provided herein. In some embodiments, the anti-TRBV19 bispecific antibody further comprises a second binding domain that binds to CD123 having a VH region of an anti-CD123 antibody provided herein. In some embodiments, the anti-TRBV19 bispecific antibody further comprises a second binding domain that binds to CD123 having a VL region of an anti-CD123 antibody provided herein. In some embodiments, the anti-TRBV19 bispecific antibody further comprises a second binding domain that binds to CD123 having a VH region of an anti-CD123 antibody provided herein, and a VL region of an anti-CD123 antibody provided herein. In some embodiments, the anti-TRBV19 bispecific antibody further comprises a second binding domain that binds to CD123 having a VH CDR1, VH CDR2, and VH CDR3 of an anti-CD123 antibody provided herein. In some embodiments, the anti-TRBV19 bispecific antibody further comprises a second binding domain that binds to CD123 having a VL CDR1, VL CDR2, and VL CDR3 of an anti-CD123 antibody provided herein. In some embodiments, the anti-TRBV19 bispecific antibody further comprises a second binding domain that binds to CD123 having a VH CDR1, VH CDR2, and VH CDR3 of an anti-CD123 antibody provided herein, and a VL CDR1, VL CDR2, and VL CDR3 of an anti-CD123 antibody provided herein.

In some embodiments, the anti-TRBV19 bispecific antibody further comprises a second binding domain that binds to ILIRAP having a VH region, VL region, VH CDR1, VH CDR2, VH CDR3, VL CDR1, VL CDR2, and/or VL CDR3 of an anti-ILIRAP antibody provided herein. In some embodiments, the anti-TRBV19 bispecific antibody further comprises a second binding domain that binds to ILIRAP having a VH region of an anti-ILIRAP antibody provided herein. In some embodiments, the anti-TRBV19 bispecific antibody further comprises a second binding domain that binds to ILIRAP having a VL region of an anti-ILIRAP antibody provided herein. In some embodiments, the anti-TRBV19 bispecific antibody further comprises a second binding domain that binds to ILIRAP having a VH region of an anti-ILIRAP antibody provided herein, and a VL region of an anti-ILIRAP antibody provided herein. In some embodiments, the anti-TRBV19 bispecific antibody further comprises a second binding domain that binds to ILIRAP having a VH CDR1, VH CDR2, and VH CDR3 of an anti-ILIRAP antibody provided herein. In some embodiments, the anti-TRBV19 bispecific antibody further comprises a second binding domain that binds to ILIRAP having a VL CDR1, VL CDR2, and VL CDR3 of an anti-ILIRAP antibody provided herein. In some embodiments, the anti-TRBV19 bispecific antibody further comprises a second binding domain that binds to ILIRAP having a VH CDR1, VH CDR2, and VH CDR3 of an anti-ILIRAP antibody provided herein, and a VL CDR1, VL CDR2, and VL CDR3 of an anti-ILIRAP antibody provided herein.

In some embodiments, the anti-TRBV19 bispecific antibody further comprises a second binding domain that binds to PSMA having a VH region, VL region, VH CDR1, VH CDR2, VH CDR3, VL CDR1, VL CDR2, and/or VL CDR3 of an anti-PSMA antibody provided herein. In some embodiments, the anti-TRBV19 bispecific antibody further comprises a second binding domain that binds to PSMA having a VH region of an anti-PSMA antibody provided herein. In some embodiments, the anti-TRBV19 bispecific antibody further comprises a second binding domain that binds to PSMA having a VL region of an anti-PSMA antibody provided herein. In some embodiments, the anti-TRBV19 bispecific antibody further comprises a second binding domain that binds to PSMA having a VH region of an anti-PSMA antibody provided herein, and a VL region of an anti-PSMA antibody provided herein. In some embodiments, the anti-TRBV19 bispecific antibody further comprises a second binding domain that binds to PSMA having a VH CDR1, VH CDR2, and VH CDR3 of an anti-PSMA antibody provided herein. In some embodiments, the anti-TRBV19 bispecific antibody further comprises a second binding domain that binds to PSMA having a VL CDR1, VL CDR2, and VL CDR3 of an anti-PSMA antibody provided herein. In some embodiments, the anti-TRBV19 bispecific antibody further comprises a second binding domain that binds to PSMA having a VH CDR1, VH CDR2, and VH CDR3 of an anti-PSMA antibody provided herein, and a VL CDR1, VL CDR2, and VL CDR3 of an anti-PSMA antibody provided herein.

In some embodiments, the anti-TRBV19 bispecific antibody further comprises a second binding domain that binds to B7H3 having a VH region, VL region, VH CDR1, VH CDR2, VH CDR3, VL CDR1, VL CDR2, and/or VL CDR3 of an anti-B7H3 antibody provided herein. In some embodiments, the anti-TRBV19 bispecific antibody further comprises a second binding domain that binds to B7H3 having a VH region of an anti-B7H3 antibody provided herein. In some embodiments, the anti-TRBV19 bispecific antibody further comprises a second binding domain that binds to B7H3 having a VL region of an anti-B7H3 antibody provided herein. In some embodiments, the anti-TRBV19 bispecific antibody further comprises a second binding domain that binds to B7H3 having a VH region of an anti-B7H3 antibody provided herein, and a VL region of an anti-B7H3 antibody provided herein. In some embodiments, the anti-TRBV19 bispecific antibody further comprises a second binding domain that binds to B7H3 having a VH CDR1, VH CDR2, and VH CDR3 of an anti-B7H3 antibody provided herein. In some embodiments, the anti-TRBV19 bispecific antibody further comprises a second binding domain that binds to B7H3 having a VL CDR1, VL CDR2, and VL CDR3 of an anti-B7H3 antibody provided herein. In some embodiments, the anti-TRBV19 bispecific antibody further comprises a second binding domain that binds to B7H3 having a VH CDR1, VH CDR2, and VH CDR3 of an anti-B7H3 antibody provided herein, and a VL CDR1, VL CDR2, and VL CDR3 of an anti-B7H3 antibody provided herein.

In some embodiments, the anti-TRBV19 bispecific antibody comprises a single chain antibody. In some embodiments, the anti-TRBV19 bispecific antibody comprises a single domain antibody. In certain embodiments, the anti-TRBV19 bispecific antibody comprises a nanobody. In certain embodiments, the anti-TRBV19 bispecific antibody comprises a VHH antibody. In certain embodiments, the anti-TRBV19 bispecific antibody comprises a llama antibody. In some embodiments, the anti-TRBV19 antibody is not a single chain antibody. In some embodiments, the anti-TRBV19 antibody is not a single domain antibody. In some embodiments, the anti-TRBV19 antibody is not a nanobody. In certain embodiments, the anti-TRBV19 antibody is not a VHH antibody. In certain embodiments, the anti-TRBV19 antibody is not a llama antibody. In some embodiments, the anti-TRBV19 bispecific antibody does not comprise a single chain antibody. In some embodiments, the anti-TRBV19 bispecific antibody does not comprise a single domain antibody. In certain embodiments, the anti-TRBV19 bispecific antibody does not comprise a nanobody. In certain embodiments, the anti-TRBV19 bispecific antibody does not comprise a VHH antibody. In certain embodiments, the anti-TRBV19 bispecific antibody does not comprise a llama antibody.

In some embodiments, the anti-TRBV19 antibody is a multispecific antibody. In other embodiments, the anti-TRBV19 is a bispecific antibody. In certain embodiments, the multispecific antibody comprises an antigen binding fragment of an anti-TRBV19 antibody provided herein. In other embodiments, the bispecific antibody comprises an antigen binding fragment of an anti-TRBV19 antibody provided herein. In some embodiments, the anti-TRBV19 antibody is an agonistic antibody. In certain embodiments, the anti-TRBV19 antibody activates T cells. In other embodiments, the anti-TRBV19 antibody is an antagonistic antibody. In certain embodiments, the anti-TRBV19 antibody inactivates T cells. In some embodiments, the anti-TRBV19 antibody blocks activation of T cells. In some embodiments, the anti-TRBV19 antibody modulates the activity of T cells. In some embodiments, the anti-TRBV19 antibody neither activates nor inactivates the activity of T cells. In specific embodiments, the T cells are human T cells. In specific embodiments, provided is a bispecific antibody comprising a TRBV19 antibody provided herein in a knob-in-hole format. In some embodiments, an anti-TRBV19 antibody provided herein may be comprised in a bispecific antibody. In some embodiments, an anti-TRBV19 bispecific antibody provided herein may be comprised in a multispecific antibody. In certain embodiments, a bispecific antibody provided herein comprises a first binding domain comprising an anti-TRBV19 antibody provided herein that binds to a first TRBV19 epitope, and a second binding domain comprising an anti-TRBV19 antibody provided herein that binds to a second TRBV19 epitope, wherein the first TRBV19 epitope and the second TRBV19 epitope are not the same. In a specific embodiment, a TRBV19 antibody, or antigen binding fragment thereof, provided herein specifically binds to TRBV19. In certain embodiments, a TRBV19 antibody, or antigen binding fragment thereof, provided herein does not bind to an epitope of TRBV19.

In some embodiments, the anti-TRBV19 antibody is a multispecific antibody. In some embodiments, the multispecific antibody comprises a first binding domain that binds to a first TRBV19 epitope and a second domain that binds to a second TRBV19 epitope, wherein the first TRBV19 epitope and the second TRBV19 epitope are different. In certain embodiments, the multispecific antibody further comprises a third binding domain that binds to a target that is not TRBV19. In another aspect, provided herein is a multispecific antibody that binds TRBV19. In some embodiments, the multispecific antibody is a bispecific antibody. In some embodiments, the multispecific antibody is a trispecific antibody. In some embodiments, the multispecific antibody is a quadraspecific antibody. In one embodiment, the multispecific TRBV19 antibody comprises: (a) a first binding domain that binds TRBV19, and (b) a second binding domain that binds to a second target. In one embodiment, the multispecific TRBV19 antibody comprises: (a) a first binding domain that binds TRBV19, and (b) a second binding domain that binds to a second target, and (c) a third binding domain that binds to a third target. In one embodiment, the multispecific TRBV19 antibody comprises: (a) a first binding domain that binds TRBV19, and (b) a second binding domain that binds to a second target, (c) a third binding domain that binds to a third target, and (d) a fourth binding domain that binds to a fourth target.

In some embodiments, the bispecific antibody provided herein comprises an Vβ17 binding arm. In some embodiments, the bispecific antibodies provided herein have high-affinity binding to Vβ17. In some embodiments, the bispecific antibodies provided herein have high specificity to Vβ17. In some embodiments, the bispecific antibodies provided herein have high-affinity binding to a second target antigen. In some embodiments, the bispecific antibodies provided herein have high-affinity binding to a second target antigen. In some embodiments, the bispecific antibodies provided herein have high specificity to a second target antigen. In some embodiments, the second target antigen is an antigen expressed on a tumor cell. In some embodiments, the bispecific antibodies provided herein have high specificity to CD123. In some, embodiments, the bispecific antibodies provided herein have high specificity to PSMA. In some embodiments, the bispecific antibodies provided herein have high specificity to ILIRAP. In some, embodiments, the bispecific antibodies provided herein have high specificity to B7H3. In some embodiments, the bispecific antibodies provided herein have the ability to treat or prevent a disease or disorder when administered alone. In some embodiments, the bispecific antibodies provided herein have the ability to treat or prevent a disease or disorder when administered in combination with other therapies. In some embodiments, the disease or disorder is a cancer. In some embodiments, the disease or disorder is a blood cancer. In some embodiments, the disease or disorder is a solid tumor cancer.

In certain embodiments, provided herein is an anti-Vβ17 bispecific antibody comprising a binding domain that binds to Vβ17 having a VH region, VL region, VH CDR1, VH CDR2, VH CDR3, VL CDR1, VL CDR2, and/or VL CDR3 of any one of the antibodies described herein. In some embodiments, provided herein is an anti-Vβ17 bispecific antibody comprising a binding domain that binds to Vβ17 having a VH region of any one of the antibodies described herein. In some embodiments, provided herein is an anti-Vβ17 bispecific antibody comprising a binding domain that binds to Vβ17 having a VL region of any one of the antibodies described herein. In some embodiments, provided herein is an anti-Vβ17 bispecific antibody comprising a binding domain that binds to Vβ17 having a VH region of any one of the antibodies described herein, and a VL region of any one of the antibodies described herein. In some embodiments, provided herein is an anti-Vβ17 bispecific antibody comprising a binding domain that binds to Vβ17 having a VH CDR1, VH CDR2, and VH CDR3 of any one of the antibodies described. In some embodiments, provided herein is an anti-Vβ17 bispecific antibody comprising a binding domain that binds to Vβ17 having a VL CDR1, VL CDR2, and VL CDR3 of any one of the antibodies described herein. In some embodiments, provided herein is an anti-Vβ17 bispecific antibody comprising a binding domain that binds to Vβ17 having a VH CDR1, VH CDR2, and VH CDR3 of any one of the antibodies described herein; and a VL CDR1, VL CDR2, and VL CDR3 of any one of the antibodies described herein. In some embodiments, the Vβ17 antibody is clone B17B1. In some embodiments, the Vβ17 antibody is clone B17B21. Other Vβ17 antibodies, including antigen binding fragments thereof, are also contemplated as the first binding arm that binds to Vβ17 of in the trispecific antibodies provided herein.

In some embodiments, the anti-Vβ17 bispecific antibody further comprises a second binding domain that binds to CD123 having a VH region, VL region, VH CDR1, VH CDR2, VH CDR3, VL CDR1, VL CDR2, and/or VL CDR3 of an anti-CD123 antibody provided herein. In some embodiments, the anti-Vβ17 bispecific antibody further comprises a second binding domain that binds to CD123 having a VH region of an anti-CD123 antibody provided herein. In some embodiments, the anti-Vβ17 bispecific antibody further comprises a second binding domain that binds to CD123 having a VL region of an anti-CD123 antibody provided herein. In some embodiments, the anti-Vβ17 bispecific antibody further comprises a second binding domain that binds to CD123 having a VH region of an anti-CD123 antibody provided herein, and a VL region of an anti-CD123 antibody provided herein. In some embodiments, the anti-Vβ17 bispecific antibody further comprises a second binding domain that binds to CD123 having a VH CDR1, VH CDR2, and VH CDR3 of an anti-CD123 antibody provided herein. In some embodiments, the anti-Vβ17 bispecific antibody further comprises a second binding domain that binds to CD123 having a VL CDR1, VL CDR2, and VL CDR3 of an anti-CD123 antibody provided herein. In some embodiments, the anti-Vβ17 bispecific antibody further comprises a second binding domain that binds to CD123 having a VH CDR1, VH CDR2, and VH CDR3 of an anti-CD123 antibody provided herein, and a VL CDR1, VL CDR2, and VL CDR3 of an anti-CD123 antibody provided herein.

In some embodiments, the anti-Vβ17 bispecific antibody further comprises a second binding domain that binds to ILIRAP having a VH region, VL region, VH CDR1, VH CDR2, VH CDR3, VL CDR1, VL CDR2, and/or VL CDR3 of an anti-ILIRAP antibody provided herein. In some embodiments, the anti-Vβ17 bispecific antibody further comprises a second binding domain that binds to ILIRAP having a VH region of an anti-ILIRAP antibody provided herein. In some embodiments, the anti-Vβ17 bispecific antibody further comprises a second binding domain that binds to ILIRAP having a VL region of an anti-ILIRAP antibody provided herein. In some embodiments, the anti-Vβ17 bispecific antibody further comprises a second binding domain that binds to ILIRAP having a VH region of an anti-ILIRAP antibody provided herein, and a VL region of an anti-ILIRAP antibody provided herein. In some embodiments, the anti-Vβ17 bispecific antibody further comprises a second binding domain that binds to ILIRAP having a VH CDR1, VH CDR2, and VH CDR3 of an anti-ILIRAP antibody provided herein. In some embodiments, the anti-Vβ17 bispecific antibody further comprises a second binding domain that binds to ILIRAP having a VL CDR1, VL CDR2, and VL CDR3 of an anti-ILIRAP antibody provided herein. In some embodiments, the anti-Vβ17 bispecific antibody further comprises a second binding domain that binds to ILIRAP having a VH CDR1, VH CDR2, and VH CDR3 of an anti-ILIRAP antibody provided herein, and a VL CDR1, VL CDR2, and VL CDR3 of an anti-ILIRAP antibody provided herein.

In some embodiments, the anti-Vβ17 bispecific antibody further comprises a second binding domain that binds to PSMA having a VH region, VL region, VH CDR1, VH CDR2, VH CDR3, VL CDR1, VL CDR2, and/or VL CDR3 of an anti-PSMA antibody provided herein. In some embodiments, the anti-Vβ17 bispecific antibody further comprises a second binding domain that binds to PSMA having a VH region of an anti-PSMA antibody provided herein. In some embodiments, the anti-Vβ17 bispecific antibody further comprises a second binding domain that binds to PSMA having a VL region of an anti-PSMA antibody provided herein. In some embodiments, the anti-Vβ17 bispecific antibody further comprises a second binding domain that binds to PSMA having a VH region of an anti-PSMA antibody provided herein, and a VL region of an anti-PSMA antibody provided herein. In some embodiments, the anti-Vβ17 bispecific antibody further comprises a second binding domain that binds to PSMA having a VH CDR1, VH CDR2, and VH CDR3 of an anti-PSMA antibody provided herein. In some embodiments, the anti-Vβ17 bispecific antibody further comprises a second binding domain that binds to PSMA having a VL CDR1, VL CDR2, and VL CDR3 of an anti-PSMA antibody provided herein. In some embodiments, the anti-Vβ17 bispecific antibody further comprises a second binding domain that binds to PSMA having a VH CDR1, VH CDR2, and VH CDR3 of an anti-PSMA antibody provided herein, and a VL CDR1, VL CDR2, and VL CDR3 of an anti-PSMA antibody provided herein.

In some embodiments, the anti-Vβ17 bispecific antibody further comprises a second binding domain that binds to B7H3 having a VH region, VL region, VH CDR1, VH CDR2, VH CDR3, VL CDR1, VL CDR2, and/or VL CDR3 of an anti-B7H3 antibody provided herein. In some embodiments, the anti-Vβ17 bispecific antibody further comprises a second binding domain that binds to B7H3 having a VH region of an anti-B7H3 antibody provided herein. In some embodiments, the anti-Vβ17 bispecific antibody further comprises a second binding domain that binds to B7H3 having a VL region of an anti-B7H3 antibody provided herein. In some embodiments, the anti-Vβ17 bispecific antibody further comprises a second binding domain that binds to B7H3 having a VH region of an anti-B7H3 antibody provided herein, and a VL region of an anti-B7H3 antibody provided herein. In some embodiments, the anti-Vβ17 bispecific antibody further comprises a second binding domain that binds to B7H3 having a VH CDR1, VH CDR2, and VH CDR3 of an anti-B7H3 antibody provided herein. In some embodiments, the anti-Vβ17 bispecific antibody further comprises a second binding domain that binds to B7H3 having a VL CDR1, VL CDR2, and VL CDR3 of an anti-B7H3 antibody provided herein. In some embodiments, the anti-Vβ17 bispecific antibody further comprises a second binding domain that binds to B7H3 having a VH CDR1, VH CDR2, and VH CDR3 of an anti-B7H3 antibody provided herein, and a VL CDR1, VL CDR2, and VL CDR3 of an anti-B7H3 antibody provided herein.

In some embodiments, the anti-Vβ17 bispecific antibody comprises a single chain antibody. In some embodiments, the anti-Vβ17 bispecific antibody comprises a single domain antibody. In certain embodiments, the anti-Vβ17 bispecific antibody comprises a nanobody. In certain embodiments, the anti-Vβ17 bispecific antibody comprises a VHH antibody. In certain embodiments, the anti-Vβ17 bispecific antibody comprises a llama antibody. In some embodiments, the anti-Vβ17 antibody is not a single chain antibody. In some embodiments, the anti-Vβ17 antibody is not a single domain antibody. In some embodiments, the anti-Vβ17 antibody is not a nanobody. In certain embodiments, the anti-Vβ17 antibody is not a VHH antibody. In certain embodiments, the anti-Vβ17 antibody is not a llama antibody. In some embodiments, the anti-Vβ17 bispecific antibody does not comprise a single chain antibody. In some embodiments, the anti-Vβ17 bispecific antibody does not comprise a single domain antibody. In certain embodiments, the anti-Vβ17 bispecific antibody does not comprise a nanobody. In certain embodiments, the anti-Vβ17 bispecific antibody does not comprise a VHH antibody. In certain embodiments, the anti-Vβ17 bispecific antibody does not comprise a llama antibody.

In some embodiments, the anti-Vβ17 antibody is a multispecific antibody. In other embodiments, the anti-Vβ17 is a bispecific antibody. In certain embodiments, the multispecific antibody comprises an antigen binding fragment of an anti-Vβ17 antibody provided herein. In other embodiments, the bispecific antibody comprises an antigen binding fragment of an anti-Vβ17 antibody provided herein. In some embodiments, the anti-Vβ17 antibody is an agonistic antibody. In certain embodiments, the anti-Vβ17 antibody activates T cells. In other embodiments, the anti-Vβ17 antibody is an antagonistic antibody. In certain embodiments, the anti-Vβ17 antibody inactivates T cells. In some embodiments, the anti-Vβ17 antibody blocks activation of T cells. In some embodiments, the anti-Vβ17 antibody modulates the activity of T cells. In some embodiments, the anti-Vβ17 antibody neither activates nor inactivates the activity of T cells. In specific embodiments, the T cells are human T cells. In specific embodiments, provided is a bispecific antibody comprising a Vβ17 antibody provided herein in a knob-in-hole format. In some embodiments, an anti-Vβ17 antibody provided herein may be comprised in a bispecific antibody. In some embodiments, an anti-Vβ17 bispecific antibody provided herein may be comprised in a multispecific antibody. In certain embodiments, a bispecific antibody provided herein comprises a first binding domain comprising an anti-Vβ17 antibody provided herein that binds to a first Vβ17 epitope, and a second binding domain comprising an anti-Vβ17 antibody provided herein that binds to a second Vβ17 epitope, wherein the first Vβ17 epitope and the second Vβ17 epitope are not the same. In a specific embodiment, a Vβ17 antibody, or antigen binding fragment thereof, provided herein specifically binds to Vβ17. In certain embodiments, a Vβ17 antibody, or antigen binding fragment thereof, provided herein does not bind to an epitope of Vβ17.

In some embodiments, the anti-Vβ17 antibody is a multispecific antibody. In some embodiments, the multispecific antibody comprises a first binding domain that binds to a first Vβ17 epitope and a second domain that binds to a second Vβ17 epitope, wherein the first Vβ17 epitope and the second Vβ17 epitope are different. In certain embodiments, the multispecific antibody further comprises a third binding domain that binds to a target that is not Vβ17. In another aspect, provided herein is a multispecific antibody that binds Vβ17. In some embodiments, the multispecific antibody is a bispecific antibody. In some embodiments, the multispecific antibody is a trispecific antibody. In some embodiments, the multispecific antibody is a quadraspecific antibody. In one embodiment, the multispecific Vβ17 antibody comprises: (a) a first binding domain that binds Vβ17, and (b) a second binding domain that binds to a second target. In one embodiment, the multispecific Vβ17 antibody comprises: (a) a first binding domain that binds Vβ17, and (b) a second binding domain that binds to a second target, and (c) a third binding domain that binds to a third target. In one embodiment, the multispecific Vβ17 antibody comprises: (a) a first binding domain that binds Vβ17, and (b) a second binding domain that binds to a second target, (c) a third binding domain that binds to a third target, and (d) a fourth binding domain that binds to a fourth target.

In some embodiments, the VH CDR1, VH CDR2, VH CDR3, VL CDR1, VL CDR2, and VL CDR3 sequences are according to the Kabat numbering system. In some embodiments, the VH CDR1, VH CDR2, VH CDR3, VL CDR1, VL CDR2, and VL CDR3 sequences are according to the Chothia numbering system. In some embodiments, the VH CDR1, VH CDR2, VH CDR3, VL CDR1, VL CDR2, and VL CDR3 sequences are according to the Exemplary numbering system. In some embodiments, the VH CDR1, VH CDR2, VH CDR3, VL CDR1, VL CDR2, and VL CDR3 sequences are according to the Contact numbering system. In some embodiments, the VH CDR1, VH CDR2, VH CDR3, VL CDR1, VL CDR2, and VL CDR3 sequences are according to the IMGT numbering system. In some embodiments, the VH CDR1, VH CDR2, VH CDR3, VL CDR1, VL CDR2, and VL CDR3 sequences are according to the AbM numbering system. Exemplary sets of 6 CDRs (VH CDR1-3 and VL CDR1-3) of certain antibody embodiments are provided herein. Other sets of CDRs are contemplated and within the scope of the antibody embodiments provided herein.

In one embodiment, the first binding domain that binds to Vβ17 comprises a VH comprising a VH CDR1, a VH CDR2, and a VH CDR3 having an amino acid sequence of a VH CDR1, VH CDR2, and VH CDR3, respectively, of SEQ ID NO:16. In one embodiment, the first binding domain that binds to Vβ17 comprises a VL CDR1, a VL CDR2, and a VL CDR3 having the amino acid sequence of a VL CDR1, VL CDR2, and VL CDR3, respectively, of SEQ ID NO:17.

SEQ ID NO: 16
QVQLQESGPGLVKPSETLSLTCTVSGYSITSGYFWNWIRQPPGKGLEWIG
YISYDGSNNYNPSLKSRVTISRDTSKNQFSLKLSSVTAADTAVYYCASPS
PGTGYAVDYWGQGTLVTVSS
SEQ ID NO: 17
DIQMTQSPSSLSASVGDRVTITCRSSQSLVHSNGNTYLHWYQQKPGKAPK
FLIYKVSNRFSGVPSRFSGSGSGTDFTLTISSLQPEDFATYYCSQSTHVP
FTFGQGTKLEIK

In one embodiment, the first binding domain that binds to Vβ17 comprises: (i) a VH comprising a VH CDR1, a VH CDR2, and a VH CDR3 having an amino acid sequence of a VH CDR1, VH CDR2, and VH CDR3, respectively, of SEQ ID NO:16; and (ii) a VL comprising a VL CDR1, a VL CDR2, and a VL CDR3 having an amino acid sequence of a VL CDR1, VL CDR2, and VL CDR3, respectively, of SEQ ID NO:17. In some embodiments, the first binding domain that binds to Vβ17, comprises a VH having the amino acid sequence of SEQ ID NO:16. In some embodiments, the first binding domain that binds to Vβ17, comprises a VL having the amino acid sequence of SEQ ID NO:17. In some embodiments, the first binding domain that binds to Vβ17, comprises a VH having the amino acid sequence of SEQ ID NO:16, and a VL having the amino acid sequence of SEQ ID NO:17. In some embodiments, the first binding domain that binds to Vβ17, comprises a VH having an amino acid sequence having at least 95% identity to the amino acid sequence of SEQ ID NO:16. In some embodiments, the first binding domain that binds to Vβ17, comprises a VL having an amino acid sequence having at least 95% identity to the amino acid sequence of SEQ ID NO:17. In some embodiments, the first binding domain that binds to Vβ17, comprises a VH having an amino acid sequence having at least 95% identity to the amino acid sequence of SEQ ID NO:16, and a VL having an amino acid sequence having at least 95% identity to the amino acid sequence of SEQ ID NO:17.

In one embodiment, the first binding domain that binds to Vβ17, comprises: (i) a VH comprising a VH CDR1 having the amino acid sequence of SEQ ID NO:7, a VH CDR2 having the amino acid sequence of SEQ ID NO:8, and a VH CDR3 having the amino acid sequence of SEQ ID NO:9; and (ii) a VL comprising a VL CDR1 having the amino acid sequence of SEQ ID NO:10, a VL CDR2 having the amino acid sequence of SEQ ID NO:11, and a VL CDR3 having the amino acid sequence of SEQ ID NO:12.

| SEQ ID NO | Sequence |
|---|---|
| 7 | GYSITSGYFWN |
| 8 | YISYDGSNN |
| 9 | PSPGTGYAVDY |
| 10 | RSSQSLVHSNGNTYLH |
| 11 | KVSNRFS |
| 12 | SQSTHVPFT |

In another aspect, provided herein is a multispecific antibody comprising: (a) a first binding domain that binds to Vβ17, and (b) a second binding domain that binds to a second target that is not Vβ17.

In some embodiments, the first binding domain comprises a heavy chain having the amino acid sequence of SEQ ID NO:18. In some embodiments, the first binding domain comprises a light chain having the amino acid sequence of SEQ ID NO:19. In some embodiments, the first binding domain comprises a heavy chain having the amino acid sequence of SEQ ID NO:18, and a light chain having the amino acid sequence of SEQ ID NO:19. In some embodiments, the first binding domain comprises a heavy chain having at least 95% identity to the amino acid sequence of SEQ ID NO:18. In some embodiments, the first binding domain comprises a light chain having at least 95% identity to the amino acid sequence of SEQ ID NO:19.

| Antibody | Heavy Chain | Light Chain |
|---|---|---|
| B17B21 | QVQLQESGPGLVKPSETLSLTC | DIQMTQSPSSLSASVGDRVTITCRSSQS |
| (Vβ17 | TVSGYSITSGYFWNWIRQPPG | LVHSNGNTYLHWYQQKPGKAPKFLIY |
| antibody) | KGLEWIGYISYDGSNNYNPSL | KVSNRFSGPSRFSGSGSGTDFTLTISS |
| | KSRVTISRDTSKNQFSLKLSSV | LQPEDFATYYCSQSTHVPFTFGQGTKL |
| | TAADTAVYYCASPSPGTGYAV | EIKRTVAAPSVFIFPPSDELQLKSGTASV |
| | DYWGQGTLVTVSSASTKGPSV | VCLLNNFYPREAKVQWKVDNALQSG |
| | FPLAPCSRSTSESTAALGCLVK | NSQESVTEQDSKDSTYSLSSTLTLSKA |
| | DYFPEPVTVSWNSGALTSGVH | DYEKHKVYACEVTHQGLSSPVTKSFNRGEC |
| | TFPAVLQSSGLYSLSSVVTVPS | (SEQ ID NO: 19) |
| | SSLGTKTYTCNVDHKPSNTKV | |
| | DKRVESKYGPPCPPCPAPEAA | |
| | GGPSVFLFPPKPKDTLMISRTP | |
| | EVTCVVVDVSQEDPEVQFNW | |
| | YVDGVEVHNAKTKPREEQFNS | |
| | TYRVVSVLTVLHQDWLNGKE | |
| | YKCKVSNKGLPSSIEKTISKAK | |
| | GQPREPQVYTLPPSQEEMTKN | |
| | QVSLSCAVKGFYPSDIAVEWE | |
| | SNGQPENNYKTTPPVLDSDGS | |
| | FFLVSRLTVDKSRWQEGNVFS | |
| | CSVMHEALHNRFTQKSLSLSL | |
| | GK (SEQ ID NO: 18) | |

In some embodiments, the second target is CD123. Thus, in another aspect, provided herein is a multispecific antibody comprising: (a) a first binding domain that binds to Vβ17, and (b) a second binding domain that binds to CD123. In some embodiments, the multispecific antibody is a bispecific antibody. In some embodiments, the first binding domain comprises the VH CDR1, VH CDR, and VH CDR3 amino acid sequences of a Vβ17 antibody provided herein. In some embodiments, the first binding domain comprises the VL CDR1, VL CDR2 and VL CDR3 amino acid sequences of a Vβ17 antibody provided herein. In some embodiments, the first binding domain comprises the VH CDR1, VH CDR2, VH CDR3, VL CDR1, VL CDR2 and VL CDR3 amino acid sequences of a Vβ17 antibody provided herein. In other embodiments, the first binding domain that binds to Vβ17 comprises a VH amino acid sequence of a Vβ17 antibody provided herein. In other embodiments, the first binding domain that binds to Vβ17 comprises a VL amino acid sequence of a Vβ17 antibody provided herein. In other embodiments, the first binding domain that binds to Vβ17 comprises VH and VL amino acid sequences of a Vβ17 antibody provided herein. In some embodiments, the second binding domain that binds to CD123 comprises VH CDR1, VH CDR2, VH CDR3, VL CDR1, VL CDR2 and VL CDR3 amino acid sequences of a CD123 antibody provided herein. In other embodiments, the second binding domain that binds to CD123 comprises a VH amino acid sequence of a CD123 antibody provided herein. In other embodiments, the second binding domain that binds to CD123 comprises a VL amino acid sequence of a CD123 antibody provided herein. In other embodiments, the second binding domain comprises VH and VL amino acid sequences of a CD123 antibody provided herein. In some embodiments, the second binding domain that binds to CD123 comprises: (i) a VH comprising a VH CDR1 having the amino acid sequence of SEQ ID NO:34, a VH CDR2 having the amino acid sequence of SEQ ID NO:35, and a VH CDR3 having the amino acid sequence of SEQ ID NO:36; and (ii) a VL comprising a VL CDR1 having the amino acid sequence of SEQ ID NO:37, a VL CDR2 having the amino acid sequence of SEQ ID NO:38, and a VL CDR3 having the amino acid sequence of SEQ ID NO:39.

| Antibody | VH CDR1 | VH CDR2 | VH CDR3 | VL CDR1 | VL CDR2 | VL CDR3 |
|---|---|---|---|---|---|---|
| I3RB217 | SYWIS | IIDPSDSD | GDGSTDLDY | RASQSVSSSYL | GASSRAT | QQDYGFPWT |
| (Anti-CD123 | (SEQ ID | TRYSPSFQG | (SEQ ID | (SEQ ID | (SEQ ID | (SEQ ID |
| mAb) | NO: 34) | (SEQ ID | NO: 36) | NO: 37) | NO: 38) | NO: 39) |
| | | NO: 35) | | | | |

| Antibody | VH | VL |
|---|---|---|
| I3RB217 | EVQLVQSGAEVKKPGESLKIS | EIVLTQSPGTLSLSPGERATLSCRASQS |
| (Anti-CD123 | CKGSGYSFTSYWISWVRQMP | VSSSYLAWYQQKPGQAPRLLIYGASSR |
| mAb) | GKGLEWMGIIDPSDSDTRYSP | ATGIPDRFSGSGSGTDFTLTISRLEPEDF |
| | SFQGQVTISADKSISTAYLQW | AVYYCQQDYGFPWTFGQGTKVEIK |
| | SSLKASDTAMYYCARGDGST | (SEQ ID NO: 41) |
| | DLDYWGQGTLVTVSS | |
| | (SEQ ID NO: 40) | |
| Antibody | Heavy Chain | Light Chain |
| I3RB217 | EVQLVQSGAEVKKPGESLKIS | EIVLTQSPGTLSLSPGERATLSCRASQS |
| (Anti-CD123 | CKGSGYSFTSYWISWVRQMP | VSSSYLAWYQQKPGQAPRLLIYGASSR |
| mAb) | GKGLEWMGIIDPSDSDTRYSP | ATGIPDRFSGSGSGTDFTLTISRLEPEDF |
| | SFQGQVTISADKSISTAYLQW | AVYYCQQDYGFPWTFGQGTKVEIKRT |
| | SSLKASDTAMYYCARGDGST | VAAPSVFIFPPSDEQLKSGTASVVCLLN |
| | DLDYWGQGTLVTVSSASTKG | NFYPREAKVQWKVDNALQSGNSQESV |
| | PSVFPLAPCSRSTSESTAALGC | TEQDSKDSTYSLSSTLTLSKADYEKHK |
| | LVKDYFPEPVTVSWNSGALTS | VYACEVTHQGLSSPVTKSFNRGEC |
| | GVHTFPAVLQSSGLYSLSSVV | (SEQ ID NO: 21) |
| | TVPSSSLGTKTYTCNVDHKPS | |
| | NTKVDKRVESKYGPPCPPCPA | |
| | PEAAGGPSVFLFPPKPKDTLM | |
| | ISRTPEVTCVVVDVSQEDPEV | |
| | QFNWYVDGVEVHNAKTKPR | |
| | EEQFNSTYRVVSVLTVLHQD | |
| | WLNGKEYKCKVSNKGLPSSIE | |
| | KTISKAKGQPREPQVYTLPPS | |
| | QEEMTKNQVSLWCLVKGFYP | |
| | SDIAVEWESNGQPENNYKTTP | |
| | PVLDSDGSFFLYSRLTVDKSR | |
| | WQEGNVFSCSVMHEALHNH | |
| | YTQKSLSLSLGK | |
| | (SEQ ID NO: 20) | |

| | | | | | Chothia | Chothia | | |
|---|---|---|---|---|---|---|---|---|
| | | Exemplary | IMGT | Kabat | v.1 | v.2 | Contact | AbM |
| VH | VH | GYSFTSY | GYSFTSY | SYWIS | GYSFTSY | GYSFTS | TSYWIS | GYSFTSY |
| CDR | CDR1 | WIS | W | (SEQ ID | WIS | YWIS | (SEQ ID | WIS |
| Seq. | | (SEQ ID | (SEQ ID | NO: 42) | (SEQ ID | (SEQ ID | NO: 60) | (SEQ ID |
| | | NO: 22) | NO: 28) | | NO: 48) | NO: 54) | | NO: 66) |
| | VH | IIDPSDSD | IDPSDSD | IIDPSDSD | IIDPSDSD | GIIDPSD | WMGIIDP | IIDPSDSD |
| | CDR2 | TRYSPSF | T | TRYSPSF | TRYSPSF | SDTRYS | SDSDTR | TR |
| | | QG | (SEQ ID | QG | QG | PSFQG | (SEQ ID | (SEQ ID |
| | | (SEQ ID | NO: 29) | (SEQ ID | (SEQ ID | (SEQ ID | NO: 61) | NO: 67) |
| | | NO: 23) | | NO: 43) | NO: 49) | NO: 55) | | |
| | VH | GDGSTD | ARGDGS | GDGSTD | GDGSTD | RGDGST | ARGDGS | GDGSTD |
| | CDR3 | LDY | TDLDY | LDY | LDY | DLDY | TDLD | LDY |
| | | (SEQ ID | (SEQ ID | (SEQ ID | (SEQ ID | (SEQ ID | (SEQ ID | (SEQ ID |
| | | NO: 24) | NO: 30) | NO: 44) | NO: 50) | NO: 56) | NO: 62) | NO: 68) |
| VL | VL | RASQSVS | QSVSSSY | SSYLA | RASQSVS | RASQSV | SSSYLA | RASQSVS |
| CDR | CDR1 | SSYLA | (SEQ ID | (SEQ ID | SSYLA | SSSYLA | WY | SSYLA |
| Seq. | | (SEQ ID | NO: 31) | NO: 45) | (SEQ ID | (SEQ ID | (SEQ ID | (SEQ ID |
| | | NO: 25) | | | NO: 51) | NO: 57) | NO: 63) | NO: 69) |
| | VL | GASSRA | GAS | GASSRA | GASSRA | GASSRA | LLIYGAS | GASSRA |
| | CDR2 | T | (SEQ ID | T | T | T | SRA | T |
| | | (SEQ ID | NO: 32) | (SEQ ID | (SEQ ID | (SEQ ID | (SEQ ID | (SEQ ID |
| | | NO: 26) | | NO: 46) | NO: 52) | NO: 58) | NO: 64) | NO: 70) |
| | VL | QQDYGF | QQDYGF | QQDYGF | QQDYGF | QQDYGF | QQDYGF | QQDYGF |
| | CDR3 | PWT | PWT | PWT | PWT | PWT | PW | PWT |
| | | (SEQ ID | (SEQ ID | (SEQ ID | (SEQ ID | (SEQ ID | (SEQ ID | (SEQ ID |
| | | NO: 27) | NO: 33) | NO: 47) | NO: 53) | NO: 59) | NO: 65) | NO: 71) |

In some embodiments, the second binding domain comprises: (i) a VH comprising a VH CDR1 having the amino acid sequence of SEQ ID NO:22, a VH CDR2 having the amino acid sequence of SEQ ID NO:23, and a VH CDR3 having the amino acid sequence of SEQ ID NO:24; and (ii) a VL comprising a VL CDR1 having the amino acid sequence of SEQ ID NO:25, a VL CDR2 having the amino acid sequence of SEQ ID NO:26, and a VL CDR3 having the amino acid sequence of SEQ ID NO:27. In some embodiments, the second binding domain comprises: (i) a VH comprising a VH CDR1 having the amino acid sequence of SEQ ID NO:28, a VH CDR2 having the amino acid sequence of SEQ ID NO:29, and a VH CDR3 having the amino acid sequence of SEQ ID NO:30; and (ii) a VL comprising a VL CDR1 having the amino acid sequence of SEQ ID NO:31, a VL CDR2 having the amino acid sequence of SEQ ID NO:32, and a VL CDR3 having the amino acid sequence of SEQ ID NO:33. In some embodiments, the second binding domain comprises: (i) a VH comprising a VH CDR1 having the amino acid sequence of SEQ ID NO:42, a VH CDR2 having the amino acid sequence of SEQ ID NO:43, and a VH CDR3 having the amino acid sequence of SEQ ID NO:44; and (ii) a VL comprising a VL CDR1 having the amino acid sequence of SEQ ID NO:45, a VL CDR2 having the amino acid sequence of SEQ ID NO:46, and a VL CDR3 having the amino acid sequence of SEQ ID NO:47. In some embodiments, the second binding domain comprises: (i) a VH comprising a VH CDR1 having the amino acid sequence of SEQ ID NO:48, a VH CDR2 having the amino acid sequence of SEQ ID NO:49, and a VH CDR3 having the amino acid sequence of SEQ ID NO:50; and (ii) a VL comprising a VL CDR1 having the amino acid sequence of SEQ ID NO:51, a VL CDR2 having the amino acid sequence of SEQ ID NO:52, and a VL CDR3 having the amino acid sequence of SEQ ID NO:53. In some embodiments, the second binding domain comprises: (i) a VH comprising a VH CDR1 having the amino acid sequence of SEQ ID NO:54, a VH CDR2 having the amino acid sequence of SEQ ID NO:55, and a VH CDR3 having the amino acid sequence of SEQ ID NO:56; and (ii) a VL comprising a VL CDR1 having the amino acid sequence of SEQ ID NO:57, a VL CDR2 having the amino acid sequence of SEQ ID NO:58, and a VL CDR3 having the amino acid sequence of SEQ ID NO:59. In some embodiments, the second binding domain comprises: (i) a VH comprising a VH CDR1 having the amino acid sequence of SEQ ID NO:60, a VH CDR2 having the amino acid sequence of SEQ ID NO:61, and a VH CDR3 having the amino acid sequence of SEQ ID NO:62; and (ii) a VL comprising a VL CDR1 having the amino acid sequence of SEQ ID NO:63, a VL CDR2 having the amino acid sequence of SEQ ID NO:64, and a VL CDR3 having the amino acid sequence of SEQ ID NO:65. In some embodiments, the second binding domain comprises: (i) a VH comprising a VH CDR1 having the amino acid sequence of SEQ ID NO:66, a VH CDR2 having the amino acid sequence of SEQ ID NO:67, and a VH CDR3 having the amino acid sequence of SEQ ID NO:68; and (ii) a VL comprising a VL CDR1 having the amino acid sequence of SEQ ID NO:69, a VL CDR2 having the amino acid sequence of SEQ ID NO:70, and a VL CDR3 having the amino acid sequence of SEQ ID NO:71.

In some embodiments, the second binding domain comprises a VH comprising a VH CDR1, a VH CDR2, and a VH CDR3 having the amino acid sequence of a VH CDR1, VH CDR2, and VH CDR3, respectively, of SEQ ID NO:40. In some embodiments, the second binding domain comprises a VL comprising a VL CDR1, a VL CDR2, and a VL CDR3 having the amino acid sequence of a VL CDR1, VL CDR2, and VL CDR3, respectively, of SEQ ID NO:41. In some embodiments, the second binding domain comprises: (i) a VH comprising a VH CDR1, a VH CDR2, and a VH CDR3 having the amino acid sequence of a VH CDR1, VH CDR2, and VH CDR3, respectively, of SEQ ID NO:40; and (ii) a VL comprising a VL CDR1, a VL CDR2, and a VL CDR3 having the amino acid sequence of a VL CDR1, VL CDR2, and VL CDR3, respectively, of SEQ ID NO:41. In some embodiments, the second binding domain comprises a VH having the amino acid sequence of SEQ ID NO:40. In some embodiments, the second binding domain comprises a VL having the amino acid sequence of SEQ ID NO:41. In some embodiments, the second binding domain comprises a VH having the amino acid sequence of SEQ ID NO:40, and a VL having the amino acid sequence of SEQ ID NO:41. In some embodiments, the second binding domain comprises a heavy chain having the amino acid sequence of SEQ ID NO:20. In some embodiments, the second binding domain comprises a light chain having the amino acid sequence of SEQ ID NO:21. In some embodiments, the second binding domain comprises a heavy chain having the amino acid sequence of SEQ ID NO:20, and a light chain having the amino acid sequence of SEQ ID NO:21. In some embodiments, the VH CDR1, VH CDR2, VH CDR3, VL CDR1, VL CDR2, and VL CDR3 sequences are according to the Kabat numbering system. In some embodiments, the VH CDR1, VH CDR2, VH CDR3, VL CDR1, VL CDR2, and VL CDR3 sequences are according to the Chothia numbering system. In some embodiments, the VH CDR1, VH CDR2, VH CDR3, VL CDR1, VL CDR2, and VL CDR3 sequences are according to the Exemplary numbering system. In some embodiments, the VH CDR1, VH CDR2, VH CDR3, VL CDR1, VL CDR2, and VL CDR3 sequences are according to the Contact numbering system. In some embodiments, the VH CDR1, VH CDR2, VH CDR3, VL CDR1, VL CDR2, and VL CDR3 sequences are according to the IMGT numbering system. In some embodiments, the VH CDR1, VH CDR2, VH CDR3, VL CDR1, VL CDR2, and VL CDR3 sequences are according to the AbM numbering system.

In some embodiments, the second target is PSMA. Thus, in another aspect, provided herein is a multispecific antibody comprising: (a) a first binding domain that binds to Vβ17, and (b) a second binding domain that binds to PSMA. In some embodiments, the multispecific antibody is a bispecific antibody. In some embodiments, the first binding domain comprises the VH CDR1, VH CDR, and VH CDR3 amino acid sequences of a Vβ17 antibody provided herein. In some embodiments, the first binding domain comprises the VL CDR1, VL CDR2 and VL CDR3 amino acid sequences of a Vβ17 antibody provided herein. In some embodiments, the first binding domain comprises the VH CDR1, VH CDR2, VH CDR3, VL CDR1, VL CDR2 and VL CDR3 amino acid sequences of a Vβ17 antibody provided herein. In other embodiments, the first binding domain that binds to Vβ17 comprises a VH amino acid sequence of a Vβ17 antibody provided herein. In other embodiments, the first binding domain that binds to Vβ17 comprises a VL amino acid sequence of a Vβ17 antibody provided herein. In other embodiments, the first binding domain that binds to Vβ17 comprises VH and VL amino acid sequences of a Vβ17 antibody provided herein. In some embodiments, the second binding domain that binds to PSMA comprises VH CDR1, VH CDR2, VH CDR3, VL CDR1, VL CDR2 and VL CDR3 amino acid sequences of a PSMA antibody provided herein. In other embodiments, the second binding domain that binds to PSMA comprises a VH amino acid sequence of a PSMA antibody provided herein. In other embodiments, the second binding domain that binds to PSMA comprises a VL amino acid sequence of a PSMA antibody provided herein. In other embodiments, the second binding domain comprises VH and VL amino acid sequences of a PSMA antibody provided herein.

PSMB365

| VH CDR1 | VH CDR2 | VH CDR3 | VL CDR1 | VL CDR2 | VL CDR3 |
|---|---|---|---|---|---|
| SDAMH | EISGSGGYT | DSYDSSLY | RASQSVSS | DASYRAT | QQRRNWP |
| (SEQ ID | NYADSLKS | V GDYFDY | YLA | | LT |
| NO: 72) | (SEQ ID | (SEQ ID | (SEQ ID | (SEQ ID | (SEQ ID |
| | NO: 73) | NO: 74) | NO: 75) | NO: 76) | NO: 77) |

| | | |
|---|---|---|
| PSMB365 | VH | EVQLLESGGGLVQPGGSLRLSCAASGFTFKSDAMHWVRQA |
| | | PGKGLEWVSEISGSGGYTNYADSLKSRFTISRDNSKNTLY |
| | | LQMNSLRAEDTAVYYCARDSYDSSLYVGDYFDYWGQGTLV |
| | | TVSS |
| | | (SEQ ID NO: 78) |
| | VL | EIVLTQSPATLSLSPGERATLSCRASQSVSSYLAWYQQKP |
| | | GQAPRLLIYDASYRATGIPARFSGSGSGTDFTLTISSLEP |
| | | EDFAVYYCQQRRNWPLTFGQGTKVEIK |
| | | (SEQ ID NO: 79) |
| | HC | EVQLLESGGGLVQPGGSLRLSCAASGFTFKSDAMHWVRQA |
| | | PGKGLEWVSEISGSGGYTNYADSLKSRFTISRDNSKNTLY |
| | | LQMNSLRAEDTAVYYCARDSYDSSLYVGDYFDYWGQGTLV |
| | | TVSSASTKGPSVFPLAPCSRSTSESTAALGCLVKDYFPEP |
| | | VTVSWNSGALTSGVHTFPAVLQSSGLYSLSSVVTVPSSSL |
| | | GTKTYTCNVDHKPSNTKVDKRVESKYGPPCPPCPAPEAAG |
| | | GPSVFLFPPKPKDTLMISRTPEVTCWVDVSQEDPEVQFNW |
| | | YVDGVEVHNAKTKPREEQFNSTYRVVSVLTVLHQDWLNGK |
| | | EYKCKVSNKGLPSSIEKTISKAKGQPREPQVYTLPPSQEE |
| | | MTKNQVSLTCLVKGFYPSDIAVEWESNGQPENNYKTTPPV |
| | | LDSDGSFFLYSRLTVDKSRWQEGNVFSCSVMHEALHNHYT |
| | | QKSLSLSLGK |
| | | (SEQ ID NO: 80) |
| | LC | EIVLTQSPATLSLSPGERATLSCRASQSVSSYLAWYQQKP |
| | | GQAPRLLIYDASYRATGIPARFSGSGSGTDFTLTISSLEP |
| | | EDFAVYYCQQRRNWPLTFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| | | (SEQ ID NO: 81) |

In some embodiments, the second binding domain that binds to PSMA comprises: (i) a VH comprising a VH CDR1 having the amino acid sequence of SEQ ID NO:72, a VH CDR2 having the amino acid sequence of SEQ ID NO:73, and a VH CDR3 having the amino acid sequence of SEQ ID NO:74; and (ii) a VL comprising a VL CDR1 having the amino acid sequence of SEQ ID NO:75, a VL CDR2 having the amino acid sequence of SEQ ID NO:76, and a VL CDR3 having the amino acid sequence of SEQ ID NO:77. In some embodiments, the second binding domain comprises a VH comprising a VH CDR1, a VH CDR2, and a VH CDR3 having the amino acid sequence of a VH CDR1, VH CDR2, and VH CDR3, respectively, of SEQ ID NO:78. In some embodiments, the second binding domain comprises a VL comprising a VL CDR1, a VL CDR2, and a VL CDR3 having the amino acid sequence of a VL CDR1, VL CDR2, and VL CDR3, respectively, of SEQ ID NO:79. In some embodiments, the second binding domain comprises: (i) a VH comprising a VH CDR1, a VH CDR2, and a VH CDR3 having an amino acid sequence of a VH CDR1, VH CDR2, and VH CDR3, respectively, of SEQ ID NO:78; and (ii) a VL comprising a VL CDR1, a VL CDR2, and a VL CDR3 having an amino acid sequence of a VL CDR1, VL CDR2, and VL CDR3, respectively, of SEQ ID NO:79. In some embodiments, the second binding domain comprises a VH having the amino acid sequence of SEQ ID NO:78. In some embodiments, the second binding domain comprises a VL having the amino acid sequence of SEQ ID NO:79. In some embodiments, the second binding domain comprises a VH having the amino acid sequence of SEQ ID NO:78, and a VL having the amino acid sequence of SEQ ID NO:79. In some embodiments, the second binding domain comprises a VH having an amino acid sequence having at least 95% identity to the amino acid sequence of SEQ ID NO:78. In some embodiments, the second binding domain comprises a VL having an amino acid sequence having at least 95% identity to the amino acid sequence of SEQ ID NO:79. In some embodiments, the second binding domain comprises a VH having an amino acid sequence having at least 95% identity to the amino acid sequence of SEQ ID NO:78, and a VL having an amino acid sequence having at least 95% identity to the amino acid sequence of SEQ ID NO:79. In some embodiments, the second binding domain comprises a heavy chain having the amino acid sequence of SEQ ID NO:80. In some embodiments, the second binding domain comprises a light chain having the amino acid sequence of SEQ ID NO:81. In some embodiments, the second binding domain comprises a heavy chain having the amino acid sequence of SEQ ID NO:80, and a light chain having the amino acid sequence of SEQ ID NO:81. In some embodiments, the VH CDR1, VH CDR2, VH CDR3, VL CDR1, VL CDR2, and VL CDR3 sequences are according to the Kabat numbering system. In some embodiments, the VH CDR1, VH CDR2, VH CDR3, VL CDR1, VL CDR2, and VL CDR3 sequences are according to the Chothia numbering system. In some embodiments, the VH CDR1, VH CDR2, VH CDR3, VL CDR1, VL CDR2, and VL CDR3 sequences are according to the Exemplary numbering system. In some embodiments, the VH CDR1, VH CDR2, VH CDR3, VL CDR1, VL CDR2, and VL CDR3 sequences are according to the Contact numbering system. In some embodiments, the VH CDR1, VH CDR2, VH CDR3, VL CDR1, VL CDR2, and VL CDR3 sequences are according to the IMGT numbering system. In some embodiments, the VH CDR1, VH CDR2, VH CDR3, VL CDR1, VL CDR2, and VL CDR3 sequences are according to the AbM numbering system.

Also provided are isolated humanized Vβ17 monoclonal antibodies or antigen-binding fragments thereof. The isolated humanized Vβ17 monoclonal antibody or antigen-binding fragment thereof can comprise an amino acid sequence with at least 95% identity to the amino acid sequence of SEQ ID NO:13. The isolated humanized Vβ17 monoclonal antibody or antigen-binding fragment thereof comprises an amino acid sequence with at least 85%, preferably 90%, more preferably 95% or more, such as 95%, 96%, 97%, 98%, or 99% identity to the amino acid sequence of SEQ ID NO:13. In certain embodiments, the isolated humanized Vβ17 monoclonal antibody or antigen-binding fragment thereof comprises the amino acid sequence of SEQ ID NO:13.

In some embodiments of the bispecific antibodies provided herein, the Vβ17 half antibody or antigen-binding fragment thereof comprises the amino acid sequence of SEQ ID NO:13.

(B17B21)
SEQ ID NO: 13
DIQMTQSPSSLSASVGDRVTITCRSSQSLVHSNGNTYLHWYQQKPGKAPK
FLIYKVSNRFSGVPSRFSGSGSGTDFTLTISSLQPEDFATYYCSQSTHVP
FTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGECGGSEGKSSGSGSESKSTEGKSSGSGSESKST
GGSQVQLQESGPGLVKPSETLSLTCTVSGYSITSGYFWNWIRQPPGKGLE
WIGYISYDGSNNYNPSLKSRVTISRDTSKNQFSLKLSSVTAADTAVYYCA
SPSPGTGYAVDYWGQGTLVTVSSASTKGPSVFPLAPCSRSTSESTAALGC
LVKDYFPEPVTVSWNSGALTSGVHTFPAVLQSSGLYSLSSVVTVPSSSLG
TKTYTCNVDHKPSNTKVDKRVESKYGPPCPPCPAPEAAGGPSVFLFPPKP
KDTLMISRTPEVTCVVVDVSQEDPEVQFNWYVDGVEVHNAKTKPREEQFN
STYRVVSVLTVLHQDWLNGKEYKCKVSNKGLPSSIEKTISKAKGQPREPQ
VYTLPPSQEEMTKNQVSLSCAVKGFYPSDIAVEWESNGQPENNYKTTPPV
LDSDGSFFLVSRLTVDKSRWQEGNVFSCSVMHEALHNRFTQKSLSLSLGK

The binding site for a second antigen can, for example, bind a cancer antigen present on the surface of a cancer cell. In some embodiments, the second antigen is CD123. In some embodiments, the second antigen is ILIRAP. In some embodiments, the second antigen is PSMA. In some embodiments, the second antigen is B7H3. The binding of the Vβ17 bispecific antibody to Vβ17 present on the surface of the T cell, and the binding of the second antigen present on the surface of the target cell can, for example, result in the killing of the target cell. The binding of the Vβ17 bispecific antibody to Vβ17 present on the surface of the T cell, and the binding of the cancer or tumor associated antigen present on the surface of the cancer cell can, for example, result in the killing of the cancer cell.

In some embodiments of the bispecific antibodies provided herein, the CD123 half antibody or antigen-binding fragment thereof comprises the amino acid sequence of SEQ ID NO:14. In some embodiments of the bispecific antibodies provided herein, the CD123 half antibody or antigen-binding fragment thereof comprises an amino acid sequence with at least 85%, preferably 90%, more preferably 95% or more, such as 95%, 96%, 97%, 98%, or 99% identity to the amino acid sequence of SEQ ID NO:14.

(I3RB217)
SEQ ID NO: 14
EIVLTQSPGTLSLSPGERATLSCRASQSVSSSYLAWYQQKPGQAPRLLIY
GASSRATGIPDRFSGSGSGTDFTLTISRLEPEDFAVYYCQQDYGFPWTFG
QGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGECGGSEGKSSGSGSESKSTEGKSSGSGSESKSTGGSE
VQLVQSGAEVKKPGESLKISCKGSGYSFTSYWISWVRQMPGKGLEWMGII
DPSDSDTRYSPSFQGQVTISADKSISTAYLQWSSLKASDTAMYYCARGDG
STDLDYWGQGTLVTVSSASTKGPSVFPLAPCSRSTSESTAALGCLVKDYF
PEPVTVSWNSGALTSGVHTFPAVLQSSGLYSLSSVVTVPSSSLGTKTYTC
NVDHKPSNTKVDKRVESKYGPPCPPCPAPEAAGGPSVFLFPPKPKDTLMI
SRTPEVTCVVVDVSQEDPEVQFNWYVDGVEVHNAKTKPREEQFNSTYRVV
SVLTVLHQDWLNGKEYKCKVSNKGLPSSIEKTISKAKGQPREPQVYTLPP
SQEEMTKNQVSLWCLVKGFYPSDIAVEWESNGQPENNYKTTPPVLDSDGS
FFLYSRLTVDKSRWQEGNVFSCSVMHEALHNHYTQKSLSLSLGK

In some embodiments of the bispecific antibodies provided herein, the PSMA half antibody or antigen-binding fragment thereof comprises the amino acid sequence of SEQ ID NO:15. In some embodiments of the bispecific antibodies provided herein, the PSMA half antibody or antigen-binding fragment thereof comprises an amino acid sequence with at least 85%, preferably 90%, more preferably 95% or more, such as 95%, 96%, 97%, 98%, or 99% identity to the amino acid sequence of SEQ ID NO:15.

(PSMB365)
SEQ ID NO: 15
EIVLTQSPATLSLSPGERATLSCRASQSVSSYLAWYQQKPGQAPRLLIYD
ASYRATGIPARFSGSGSGTDFTLTISSLEPEDFAVYYCQQRRNWPLTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGECGGSEGKSSGSGSESKSTEGKSSGSGSESKSTGGSEV
QLLESGGGLVQPGGSLRLSCAASGFTFKSDAMHWVRQAPGKGLEWVSEIS
GSGGYTNYADSLKSRFTISRDNSKNTLYLQMNSLRAEDTAVYYCARDSYD
SSLYVGDYFDYWGQGTLVTVSSASTKGPSVFPLAPCSRSTSESTAALGCL
VKDYFPEPVTVSWNSGALTSGVHTFPAVLQSSGLYSLSSVVTVPSSSLGT
KTYTCNVDHKPSNTKVDKRVESKYGPPCPPCPAPEAAGGPSVFLFPPKPK
DTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQV
YTLPPSQEEMTKNQVSLWCLVKGFYPSDIAVEWESNGQPENNYKTTPPVL
DSDGSFFLYSRLTVDKSRWQEGNVFSCSVMHEALHNHYTQKSLSLSLGK

In some embodiments of the bispecific antibodies provided herein, the ILIRAP half antibody or antigen-binding fragment thereof comprises the amino acid sequence of SEQ ID NO:82. In some embodiments of the bispecific antibodies provided herein, the ILIRAP half antibody or antigen-binding fragment thereof comprises an amino acid sequence with at least 85%, preferably 90%, more preferably 95% or more, such as 95%, 96%, 97%, 98%, or 99% identity to the amino acid sequence of SEQ ID NO:82.

(IAPB63)
SEQ ID NO: 82
QSALTQPRSVSGSPGHSVTISCTGTSSDVGDYNYVSWYQQRPGKVPKLLI
YDVSKRPSGVPDRFSGSKSGNTASLTISGLQAEDEAIYFCASYAGNYNVV
FGGGTKLTVLGQPKAAPSVTLFPPSSEELQANKATLVCLISDFYPGAVTV
AWKADSSPVKAGVETTTPSKQSNNKYAASSYLSLTPEQWKSHRSYSCQVT
HEGSTVEKTVAPTECSGGSEGKSSGSGSESKSTEGKSSGSGSESKSTGGS
QVQLVQSGSELKKPGASVKVSCKASGYTFNTYAMNWVRQAPGQGLEWMGW
INTNTGNPTYAQGFTGRFVFSLDTSVSTAYLQISSLKAEDTAVYYCARRY
FDWLLGAFDIWGQGTMVTVSSASTKGPSVFPLAPCSRSTSESTAALGCLV
KDYFPEPVTVSWNSGALTSGVHTFPAVLQSSGLYSLSSVVTVPSSSLGTK
TYTCNVDHKPSNTKVDKRVESKYGPPCPPCPAPEAAGGPSVFLFPPKPKD
TLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVY
TLPPSQEEMTKNQVSLWCLVKGFYPSDIAVEWESNGQPENNYKTTPPVLD
SDGSFFLYSRLTVDKSRWQEGNVFSCSVMHEALHNHYTQKSLSLSLGK

In another aspect, provided is an antibody that competes for binding to TRBV19 with a TRBV19 reference antibody. In another aspect, provided is a TRBV19 antibody that binds to the same TRBV19 epitope as a TRBV19 reference antibody. In another aspect, provided is a TRBV19 antibody that binds an epitope on TRBV19 that overlaps with the epitope on TRBV19 bound by a TRBV19 reference antibody. In some embodiments, the TRBV19 reference antibody comprises a VH CDR1, VH CDR2, and VH CDR3 of a TRBV19 reference antibody provided herein. In some embodiments, the TRBV19 reference antibody comprises a VL CDR1, VL CDR2, and VL CDR3 of a TRBV19 reference antibody provided herein. In some embodiments, the TRBV19 reference antibody comprises a VH CDR1, VH CDR2, VH CDR3, a VL CDR1, VL CDR2, and VL CDR3 of a TRBV19 reference antibody provided herein. In some embodiments, the TRBV19 reference antibody comprises a VH of a TRBV19 reference antibody provided herein. In some embodiments, the TRBV19 reference antibody comprises a VL of a TRBV19 reference antibody provided herein. In some embodiments, the TRBV19 reference antibody comprises a VH and a VL of a TRBV19 reference antibody provided herein. In some embodiments, the TRBV19 reference antibody comprises a VH CDR1, VH CDR2, VH CDR3, a VL CDR1, VL CDR2, and VL CDR3 of a TRBV19 reference antibody provided herein. In some embodiments, the VH CDR1, VH CDR2, VH CDR3, VL CDR1, VL CDR2, and VL CDR3 amino acid sequences of the TRBV19 reference antibody are according to the Kabat numbering system. In some embodiments, the VH CDR1, VH CDR2, VH CDR3, VL CDR1, VL CDR2, and VL CDR3 amino acid sequences of the TRBV19 reference antibody are according to the Chothia numbering system. In some embodiments, the VH CDR1, VH CDR2, VH CDR3, VL CDR1, VL CDR2, and VL CDR3 amino acid sequences of the TRBV19 reference antibody are according to the AbM numbering system. In some embodiments, the VH CDR1, VH CDR2, VH CDR3, VL CDR1, VL CDR2, and VL CDR3 amino acid sequences of the TRBV19 reference antibody are according to the Contact numbering system. In some embodiments, the VH CDR1, VH CDR2, VH CDR3, VL CDR1, VL CDR2, and VL CDR3 amino acid sequences of the TRBV19 reference antibody are according to the IMGT numbering system. In certain embodiments, the antibody is a multispecific antibody. In some embodiments, the antibody is a bispecific antibody. In certain embodiments, the TRBV19 reference antibody is a multispecific antibody. In some embodiments, the TRBV19 reference antibody is a bispecific antibody.

In another aspect, provided is an antibody that competes for binding to Vβ17 with a Vβ17 reference antibody. In another aspect, provided is a Vβ17 antibody that binds to the same Vβ17 epitope as a Vβ17 reference antibody. In another aspect, provided is a Vβ17 antibody that binds an epitope on Vβ17 that overlaps with the epitope on Vβ17 bound by a Vβ17 reference antibody. In some embodiments, the Vβ17 reference antibody comprises a VH CDR1, VH CDR2, and VH CDR3 of a Vβ17 reference antibody provided herein. In some embodiments, the Vβ17 reference antibody comprises a VL CDR1, VL CDR2, and VL CDR3 of a Vβ17 reference antibody provided herein. In some embodiments, the Vβ17 reference antibody comprises a VH CDR1, VH CDR2, VH CDR3, a VL CDR1, VL CDR2, and VL CDR3 of a Vβ17 reference antibody provided herein. In some embodiments, the Vβ17 reference antibody comprises a VH of a Vβ17 reference antibody provided herein. In some embodiments, the Vβ17 reference antibody comprises a VL of a Vβ17 reference antibody provided herein. In some embodiments, the Vβ17 reference antibody comprises a VH and a VL of a Vβ17 reference antibody provided herein. In some embodiments, the Vβ17 reference antibody comprises a VH CDR1, VH CDR2, VH CDR3, a VL CDR1, VL CDR2, and VL CDR3 of a Vβ17 reference antibody provided herein. In some embodiments, the VH CDR1, VH CDR2, VH CDR3, VL CDR1, VL CDR2, and VL CDR3 amino acid sequences of the Vβ17 reference antibody are according to the Kabat numbering system. In some embodiments, the VH CDR1, VH CDR2, VH CDR3, VL CDR1, VL CDR2, and VL CDR3 amino acid sequences of the Vβ17 reference antibody are according to the Chothia numbering system. In some embodiments, the VH CDR1, VH CDR2, VH CDR3, VL CDR1, VL CDR2, and VL CDR3 amino acid sequences of the Vβ17 reference antibody are according to the AbM numbering system. In some embodiments, the VH CDR1, VH CDR2, VH CDR3, VL CDR1, VL CDR2, and VL CDR3 amino acid sequences of the Vβ17 reference antibody are according to the Contact numbering system. In some embodiments, the VH CDR1, VH CDR2, VH CDR3, VL CDR1, VL CDR2, and VL CDR3 amino acid sequences of the Vβ17 reference antibody are according to the IMGT numbering system. In certain embodiments, the antibody is a multispecific antibody. In some embodiments, the antibody is a bispecific antibody. In certain embodiments, the Vβ17 reference antibody is a multispecific antibody. In some embodiments, the Vβ17 reference antibody is a bispecific antibody.

### 5.5. Pharmaceutical Compositions or Combination

In one aspect, the present disclosure further provides pharmaceutical compositions comprising an engineered T cell of the present disclosure. In some embodiments, the present disclosure provides a pharmaceutical combination comprising: (i) an isolated population of cells comprising Vβ17+CD8+ T cells, and (ii) one or more T cell engagers, wherein (i) and (ii) are present in the same pharmaceutical composition or in two separate pharmaceutical compositions. In some embodiments, (i) and (ii) are present in two separate pharmaceutical compositions, each further independently comprise a pharmaceutically acceptable excipient. The two pharmaceutical compositions can be provided together or separately for use in combination. For example, (i) and (ii) can be provided in separate compositions in a kit. In other embodiments, (i) and (ii) are present in one composition. In some embodiments, a pharmaceutical composition comprises (i) an isolated population of cells comprising Vβ17⁺CD8⁺ T cells provided herein and (ii) one or more T cell engagers provided herein. In some embodiments, the pharmaceutical composition further comprises a pharmaceutically acceptable excipient.

In a specific embodiment, the term “excipient” can also refer to a diluent, adjuvant (e.g., Freunds' adjuvant (complete or incomplete), carrier or vehicle. Pharmaceutical excipients can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid excipients. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical excipients are described in Remington's Pharmaceutical Sciences (1990) Mack Publishing Co., Easton, PA. Such compositions will contain a prophylactically or therapeutically effective amount of the active ingredient provided herein, such as in purified form, together with a suitable amount of excipient so as to provide the form for proper administration to the patient. The formulation should suit the mode of administration.

In some embodiments, the choice of excipient is determined in part by the particular cell, and/or by the method of administration. Accordingly, there are a variety of suitable formulations.

Typically, acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers, stabilizers, metal complexes (e.g. Zn-protein complexes); chelating agents such as EDTA and/or non-ionic surfactants.

Buffers may be used to control the pH in a range which optimizes the therapeutic effectiveness, especially if stability is pH dependent. Suitable buffering agents for use with the present disclosure include both organic and inorganic acids and salts thereof. For example, citrate, phosphate, succinate, tartrate, fumarate, gluconate, oxalate, lactate, acetate. Additionally, buffers may comprise histidine and trimethylamine salts such as Tris.

Preservatives may be added to retard microbial growth. Suitable preservatives for use with the present disclosure include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” can be present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intra-molecular interactions. Exemplary tonicity agents include polyhydric sugar alcohols, trihydric or higher sugar alcohols, such as glycerin, erythritol, arabitol, xylitol, sorbitol and mannitol.

Additional exemplary excipients include: (1) bulking agents, (2) solubility enhancers, (3) stabilizers and (4) agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, α-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) may be present to help solubilize the therapeutic agent as well as to protect the therapeutic protein against agitation-induced aggregation, which also permits the formulation to be exposed to shear surface stress without causing denaturation of the active therapeutic protein or antibody. Suitable non-ionic surfactants include, e.g., polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

In order for the pharmaceutical compositions to be used for in vivo administration, they are preferably sterile. The pharmaceutical composition may be rendered sterile by filtration through sterile filtration membranes. The pharmaceutical compositions herein generally can be placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

In another embodiment, a pharmaceutical composition can be provided as a controlled release or sustained release system. In one embodiment, a pump may be used to achieve controlled or sustained release (see, e.g., Sefton, Crit. Ref. Biomed. Eng. 14:201-40 (1987); Buchwald et al., Surgery 88:507-16 (1980); and Saudek et al., N. Engl. J. Med. 321:569-74 (1989)). In another embodiment, polymeric materials can be used to achieve controlled or sustained release of a prophylactic or therapeutic agent (e.g., a fusion protein as described herein) or a composition provided herein (see, e.g., Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 23:61-126 (1983); Levy et al., Science 228:190-92 (1985); During et al., Ann. Neurol. 25:351-56 (1989); Howard et al., J. Neurosurg. 71:105-12 (1989); U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable. In yet another embodiment, a controlled or sustained release system can be placed in proximity of a particular target tissue, for example, the nasal passages or lungs, thus requiring only a fraction of the systemic dose (see, e.g., Goodson, Medical Applications of Controlled Release Vol. 2, 115-38 (1984)). Controlled release systems are discussed, for example, by Langer, Science 249:1527-33 (1990). Any technique known to one of skill in the art can be used to produce sustained release formulations comprising one or more agents as described herein (see, e.g., U.S. Pat. No. 4,526,938, PCT publication Nos. WO 91/05548 and WO 96/20698, Ning et al., Radiotherapy & Oncology 39:179-89 (1996); Song et al., PDA J. of Pharma. Sci. & Tech. 50:372-97 (1995); Cleek et al., Pro. Int'l. Symp. Control. Rel. Bioact. Mater. 24:853-54 (1997); and Lam et al., Proc. Int'l. Symp. Control Rel. Bioact. Mater. 24:759-60 (1997)).

The pharmaceutical compositions described herein may also contain more than one active compound or agent as necessary for the particular indication being treated. Alternatively, or in addition, the composition may comprise a cytotoxic agent, chemotherapeutic agent, cytokine, immunosuppressive agent, or growth inhibitory agent. Such molecules are suitably present in combination in amounts that are effective for the purpose intended.

The active ingredients may also be entrapped in microcapsules prepared, for example, by coascervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18th edition.

Various compositions and delivery systems are known and can be used with the therapeutic agents provided herein, including, but not limited to, encapsulation in liposomes, microparticles, microcapsules, recombinant cells capable of expressing the single domain antibody or therapeutic molecule provided herein, construction of a nucleic acid as part of a retroviral or other vector, etc.

In some embodiments, the pharmaceutical composition provided herein contains the binding molecules and/or cells in amounts effective to treat or prevent the disease or disorder, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined.

### 5.6. Methods and Uses

In another aspect, provided herein are methods for using and uses of the engineered Vβ17⁺CD8⁺ T cells provided herein in combination with T cell engagers as those described in Section 5.4 above. In some embodiments, the Vβ17⁺CD8⁺ T cells are used in a method for redirecting a T cell to a target cell, comprising contacting the target cell with one or more T cell engagers in the presence of an isolated population of cells comprising Vβ17+CD8+ T cells, wherein the contacting directs the Vβ17+CD8+ T cell to the target cell. In some embodiments, the Vβ17⁺CD8⁺ T cells are used in a method for inhibiting the growth or proliferation of a target cell, comprising contacting the target cell with one or more T cell engagers in the presence of an isolated population of cells comprising Vβ17+CD8+ T cells, wherein the contacting results in the inhibition of the growth or proliferation of the target cell. In some embodiments, the Vβ17⁺CD8⁺ T cells are used in a method for eliminating a target cell, comprising contacting the target cell with one or more T cell engagers in the presence of an isolated population of cells comprising Vβ17+CD8+ T cells, wherein the contacting results in the elimination of the target cell. In some embodiments, the Vβ17⁺CD8⁺ T cells are used in a method for treating a disease or disorder in a subject, comprising administering to the subject: (i) a therapeutically effective amount of an isolated population of cells comprising Vβ17+CD8+ T cells, and (ii) a therapeutically effective amount of one or more T cell engagers.

In some embodiments, the engineered T cells provided herein are useful as allogenic T cell therapies. In some embodiments, the present T cell therapy has more safety features that are absent from the traditional autologous T therapy, for example, no or low cytokine storm, no stimulation of regulatory T cells, reduced self-tissue damage, reduced induction of autoimmunity, reduced graft-versus-host disease, etc.

Such methods and uses include therapeutic methods and uses, for example, involving administration of the cells, or compositions containing the same, to a subject having a disease or disorder. In some embodiments, the cell is administered in an effective amount to effect treatment of the disease or disorder. Uses include uses of the cells in such methods and treatments, and in the preparation of a medicament in order to carry out such therapeutic methods. In some embodiments, the methods are carried out by administering the cells, or compositions comprising the same, to the subject having or suspected of having the disease or condition. In some embodiments, the methods thereby treat the disease or disorder in the subject.

In some embodiments, the treatment provided herein cause complete or partial amelioration or reduction of a disease or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms include, but do not imply, complete curing of a disease or complete elimination of any symptom or effect(s) on all symptoms or outcomes.

As used herein, in some embodiments, the treatment provided herein delay development of a disease or disorder, e.g., defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease or disorder. For example, a late stage cancer, such as development of metastasis, may be delayed. In other embodiments, the method or the use provided herein prevents a disease or disorder.

In some embodiments, the present T cell therapies are used for treating solid tumor cancer. In other embodiments, the present T cell therapies are used for treating blood cancer. In other embodiments, the disease or disorder is an autoimmune and inflammatory disease.

In some embodiments, the methods include adoptive cell therapy, whereby genetically engineered cells are administered to a subject. Such administration can promote activation of the cells (e.g., T cell activation), such that the cells of the disease or disorder are targeted for destruction.

In some embodiments, the methods include administration of the cells or a composition containing the cells to a subject, tissue, or cell, such as one having, at risk for, or suspected of having the disease or disorder. In some embodiments, the cells, populations, and compositions are administered to a subject having the particular disease or disorder to be treated, e.g., via adoptive cell therapy, such as adoptive T cell therapy. In some embodiments, the cells or compositions are administered to the subject, such as a subject having or at risk for the disease or disorder. In some embodiments, the methods thereby treat, e.g., ameliorate one or more symptom of the disease or disorder.

Methods for administration of cells for adoptive cell therapy are known, as described, e.g., in US Patent Application Publication No. 2003/0170238; U.S. Pat. No. 4,690,915; Rosenberg, Nat Rev Clin Oncol. 8 (10):577-85 (2011); Themeli et al., Nat Biotechnol. 31(10): 928-933 (2013); Tsukahara et al., Biochem Biophys Res Commun 438(1): 84-9 (2013); and Davila et al., PLoS ONE 8(4): e61338 (2013). These methods may be used in connection with the methods and compositions provided herein.

In some embodiments, the cell therapy (e.g., adoptive T cell therapy) is carried out by autologous transfer, in which the cells are isolated and/or otherwise prepared from the subject who is to receive the cell therapy, or from a sample derived from such a subject. Thus, in some aspects, the cells are derived from a subject in need of a treatment and the cells, following isolation and processing are administered to the same subject. In other embodiments, the cell therapy (e.g., adoptive T cell therapy) is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject, to whom the cells, cell populations, or compositions are administered is a primate, such as a human. The subject can be male or female and can be any suitable age, including infant, juvenile, adolescent, adult, and geriatric subjects. In some examples, the subject is a validated animal model for disease, adoptive cell therapy, and/or for assessing toxic outcomes.

The composition provided herein can be administered by any suitable means, for example, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration.

The amount of a prophylactic or therapeutic agent provided herein that will be effective in the prevention and/or treatment of a disease or condition can be determined by standard clinical techniques. Effective doses may be extrapolated from dose-response curves derived from in vitro or animal model test systems. For the prevention or treatment of disease, the appropriate dosage of the binding molecule or cell may depend on the type of disease or disorder to be treated, the type of binding molecule, the severity and course of the disease or disorder, whether the therapeutic agent is administered for preventive or therapeutic purposes, previous therapy, the patient's clinical history and response to the agent, and the discretion of the attending physician. The compositions, molecules and cells are in some embodiments suitably administered to the patient at one time or over a series of treatments. Multiple doses may be administered intermittently. An initial higher loading dose, followed by one or more lower doses may be administered.

In the context of genetically engineered cells, in some embodiments, a subject may be administered the range of about one million to about 100 billion cells and/or that amount of cells per kilogram of body weight. In some embodiments, wherein the pharmaceutical composition comprises any one of the engineered immune cells described herein, the pharmaceutical composition is administered at a dosage of at least about any of 104, 105, 106, 107, 108, or 109 cells/kg of body weight of the individual. Dosages may vary depending on attributes particular to the disease or disorder and/or patient and/or other treatments.

In some embodiments, the pharmaceutical composition is administered for a single time. In some embodiments, the pharmaceutical composition is administered for multiple times (such as any of 2, 3, 4, 5, 6, or more times). In some embodiments, the pharmaceutical composition is administered once or multiple times during a dosing cycle. A dosing cycle can be, e.g., 1, 2, 3, 4, 5 or more week(s), or 1, 2, 3, 4, 5, or more month(s). The optimal dosage and treatment regime for a particular patient can be determined by one skilled in the art of medicine by monitoring the patient for signs of disease and adjusting the treatment accordingly.

In some embodiments, the compositions provided herein are administered as part of a combination treatment, such as simultaneously with or sequentially with, in any order, another therapeutic intervention, such as another antibody or engineered cell or receptor or agent, such as a cytotoxic or therapeutic agent.

In some embodiments, the compositions provided herein are co-administered with one or more additional therapeutic agents or in connection with another therapeutic intervention, either simultaneously or sequentially in any order. In some embodiments, the cells are co-administered with another therapy sufficiently close in time such that the cell populations enhance the effect of one or more additional therapeutic agents, or vice versa. In some embodiments, the compositions provided herein are administered prior to the one or more additional therapeutic agents. In some embodiments, the compositions provided herein are administered after to the one or more additional therapeutic agents.

In certain embodiments, once the cells are administered to a mammal (e.g., a human), the biological activity of the engineered cell populations is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as CD107a, IFNγ, IL-2, and TNF. In some aspects the biological activity is measured by assessing clinical outcome, such as reduction in tumor burden or load.

### 5.7. Kits and Articles of Manufacture

Further provided are kits, unit dosages, and articles of manufacture comprising any of the engineered immune effector cells described herein. In some embodiments, a kit is provided which contains any one of the pharmaceutical compositions described herein and preferably provides instructions for its use.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder (such as cancer) described herein, and may have a sterile access port (for example the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual. The label may indicate directions for reconstitution and/or use. The container holding the pharmaceutical composition may be a multi-use vial, which allows for repeat administrations (e.g. from 2-6 administrations) of the reconstituted formulation. Package insert refers to instructions customarily included in commercial packages of therapeutic products that contain information about the indications, usage, dosage, administration, contraindications and/or warnings concerning the use of such therapeutic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the pharmaceutical composition and instructions for use, packaged in quantities sufficient for storage and use in pharmacies, for example, hospital pharmacies and compounding pharmacies.

For the sake of conciseness, certain abbreviations are used herein. One example is the single letter abbreviation to represent amino acid residues. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

The disclosure is generally disclosed herein using affirmative language to describe the numerous embodiments. The disclosure also specifically includes embodiments in which particular subject matter is excluded, in full or in part, such as substances or materials, method steps and conditions, protocols, procedures, assays or analysis. Thus, even though the disclosure is generally not expressed herein in terms of what the disclosure does not include, aspects that are not expressly included in the disclosure are nevertheless disclosed herein.

A number of embodiments of the disclosure have been described. Nevertheless, it will be understood that various modifications may be made without departing from the spirit and scope of the disclosure. Accordingly, the following examples are intended to illustrate but not limit the scope of disclosure described in the claims.

### 6. EMBODIMENTS

This invention provides the following non-limiting embodiments.

In one set of embodiments (embodiment set A), provided are:
A. A pharmaceutical combination comprising: (i) an isolated population of cells comprising Vβ17⁺CD8⁺ T cells, and (ii) one or more T cell engagers, wherein (i) and (ii) are present in the same pharmaceutical composition or in two separate pharmaceutical compositions.
A1. A pharmaceutical composition comprising: (i) an isolated population of cells comprising Vβ17+CD8⁺ T cells, (ii) one or more T cell engagers; and (iii) a pharmaceutically acceptable excipient.
A2. The pharmaceutical composition of embodiment A1, wherein the isolated population of cells comprising Vβ17+CD8+ T cells has been activated or enriched via contacting a M1 peptide derived from human influenza A virus (M1₅₈₋₆₆) with a population of cells comprising T cells.
A3. The pharmaceutical composition of embodiment A2, wherein the M1 peptide comprises an amino acid sequence of GILGFVFTL (SEQ ID NO:1).
A4. The pharmaceutical composition of any one of embodiments A1 to A3, wherein the isolated population of cells comprising Vβ17+CD8+ T cells has been activated or enriched via further contacting IL-2 with the population of cells comprising T cells.
A5. The pharmaceutical composition of any one of embodiments A2 to A4, wherein the population of cells comprising T cells has been cultured ex vivo in a medium comprising the M1 peptide and/or IL-2.
A6. The pharmaceutical composition of embodiment A5, wherein the ex vivo culturing comprises:
   (i). culturing the population of cells comprising T cells ex vivo in a medium comprising the M1 peptide and IL-2;
   (ii). culturing the population of cells comprising T cells ex vivo in a medium comprising the M1 peptide, and then culturing the population of cells comprising T cells ex vivo in a medium comprising IL-2; or
   (iii). culturing the population of cells comprising T cells ex vivo in a medium comprising the M1 peptide, then culturing the population of cells comprising T cells ex vivo in a medium comprising the M1 peptide and IL-2; and then culturing the population of cells comprising T cells ex vivo in a medium comprising IL-2.
A7. The pharmaceutical composition of embodiment A5 or A6, wherein the population of cells comprising T cells has been cultured ex vivo for at least 1, 2, 3, 4, 5, 6, 7, 8, 9, 10, 11, 12, 13, or 14 days.
A8. The pharmaceutical composition of any one of embodiments A2 to A7, wherein the population of cells comprising T cells is a population of whole peripheral blood mononuclear cells (PBMCs).
A9. The pharmaceutical composition of embodiment A8, wherein the population of the whole PBMCs is from a healthy donor or an unhealthy donor.
A10. The pharmaceutical composition of any one of embodiments A1 to A9, wherein the Vβ17+CD8+ T cells have been isolated from the population of cells comprising T cells after contacting the population of cells comprising T cells with the M1 peptide and/or IL-2.
A11. The pharmaceutical composition of any one of embodiments A1 to A10, wherein the percent of Vβ17+CD8+ T cells in the isolated population of cells is more than 6%, 10%, 15%, 20%, 25%, 30%, 35%, 40%, 45%, 50%, 55%, 60%, 65%, 70%, 75%, 80%, 85%, or 90%.
A12. The pharmaceutical composition of any one of embodiments A1 to A11, wherein at least part of the Vβ17⁺CD8⁺ T cells express a cell surface receptor capable of binding to the M1 peptide.
A13. The pharmaceutical composition of any one of embodiments A1 to A12, wherein each of the T cell engagers is a multi-specific antibody.
A14. The pharmaceutical composition of any one of embodiments A1 to A13, wherein each of the T cell engagers comprises:
   1) a first binding domain that binds to an antigen expressed on a Vβ17+CD8+ T cell; and
   2) a second binding domain that binds to an antigen expressed on an unhealthy cell.
A15. The pharmaceutical composition of embodiment A14, wherein the antigen expressed on the Vβ17+CD8+ T cell is T Cell Receptor Beta Variable 19 (TRBV19) or Vβ17.
A15-1. The pharmaceutical composition of embodiment A14, wherein the first binding domain comprises an antibody or an antigen-binding fragment thereof comprising: (i) a VH comprising a VH CDR1, a VH CDR2, and a VH CDR3 having an amino acid sequence of a VH CDR1, VH CDR2, and VH CDR3, respectively, of SEQ ID NO:16; and (ii) a VL comprising a VL CDR1, a VL CDR2, and a VL CDR3 having an amino acid sequence of a VL CDR1, VL CDR2, and VL CDR3, respectively, of SEQ ID NO:17.
A15-2. The pharmaceutical composition of embodiment A15-1, wherein the first binding domain comprises an antibody or an antigen-binding fragment thereof comprising: (i) a VH comprising a VH CDR1 having the amino acid sequence of SEQ ID NO:7, a VH CDR2 having the amino acid sequence of SEQ ID NO:8, and a VH CDR3 having the amino acid sequence of SEQ ID NO:9; and (ii) a VL comprising a VL CDR1 having the amino acid sequence of SEQ ID NO:10, a VL CDR2 having the amino acid sequence of SEQ ID NO:11, and a VL CDR3 having the amino acid sequence of SEQ ID NO:12.
A15-3. The pharmaceutical composition of embodiment A14, wherein the second binding domain comprises an antibody or an antigen-binding fragment thereof comprising: (i) a VH comprising a VH CDR1, a VH CDR2, and a VH CDR3 having an amino acid sequence of a VH CDR1, VH CDR2, and VH CDR3, respectively, of SEQ ID NO:40; and (ii) a VL comprising a VL CDR1, a VL CDR2, and a VL CDR3 having an amino acid sequence of a VL CDR1, VL CDR2, and VL CDR3, respectively, of SEQ ID NO:41.
A15-4. The pharmaceutical composition of embodiment A15-3, wherein the second binding domain comprises an antibody or an antigen-binding fragment thereof comprising: (i) a VH comprising a VH CDR1 having the amino acid sequence of SEQ ID NO:34, a VH CDR2 having the amino acid sequence of SEQ ID NO:35, and a VH CDR3 having the amino acid sequence of SEQ ID NO:36; and (ii) a VL comprising a VL CDR1 having the amino acid sequence of SEQ ID NO:37, a VL CDR2 having the amino acid sequence of SEQ ID NO:38, and a VL CDR3 having the amino acid sequence of SEQ ID NO:39.
A15-5. The pharmaceutical composition of embodiment A14, wherein the second binding domain comprises an antibody or an antigen-binding fragment thereof comprising: (i) a VH comprising a VH CDR1, a VH CDR2, and a VH CDR3 having an amino acid sequence of a VH CDR1, VH CDR2, and VH CDR3, respectively, of SEQ ID NO:78; and (ii) a VL comprising a VL CDR1, a VL CDR2, and a VL CDR3 having an amino acid sequence of a VL CDR1, VL CDR2, and VL CDR3, respectively, of SEQ ID NO:79.
A15-6. The pharmaceutical composition of embodiment A15-5, wherein the second binding domain comprises an antibody or an antigen-binding fragment thereof comprising: (i) a VH comprising a VH CDR1 having the amino acid sequence of SEQ ID NO:72, a VH CDR2 having the amino acid sequence of SEQ ID NO:73, and a VH CDR3 having the amino acid sequence of SEQ ID NO:74; and (ii) a VL comprising a VL CDR1 having the amino acid sequence of SEQ ID NO:75, a VL CDR2 having the amino acid sequence of SEQ ID NO:76, and a VL CDR3 having the amino acid sequence of SEQ ID NO:77.
A16. The pharmaceutical composition of embodiment A14 or A15, wherein the unhealthy cell is a cancer cell.
A17. The pharmaceutical composition of embodiment A16, wherein the cancer cell is a blood cancer cell or a solid tumor cancer cell.
A18. The pharmaceutical composition of any one of embodiments A14 to A17, and wherein the antigen expressed on the unhealthy cell is a tumor-associated antigen (TAA).
A19. The pharmaceutical composition of embodiment A18, wherein the TAA is CD123, ILIRAP, PSMA, or B7H3.
A20. The pharmaceutical composition of any one of embodiments A1 to A19, wherein each of the T cell engagers is a TRBV19xTAA or Vβ17xTAA bi-specific antibody.
A1a. A pharmaceutical combination comprising: (i) an isolated population of cells comprising Vβ17+CD8+ T cells, and (ii) one or more T cell engagers, wherein (i) and (ii) are present in two separate pharmaceutical compositions.
A2a. The pharmaceutical combination of embodiment A1a, wherein the isolated population of cells comprising Vβ17+CD8+ T cells has been activated or enriched via contacting a M1 peptide derived from human influenza A virus (M1₅₈₋₆₆) with a population of cells comprising T cells.
A3a. The pharmaceutical combination of embodiment A2a, wherein the M1 peptide comprises an amino acid sequence of GILGFVFTL (SEQ ID NO:1).
A4a. The pharmaceutical combination of any one of embodiments A1a to A3a, wherein the isolated population of cells comprising Vβ17⁺CD8⁺ T cells has been activated or enriched via further contacting IL-2 with the population of cells comprising T cells.
A5a. The pharmaceutical combination of any one of embodiments A2a to A4a, wherein the population of cells comprising T cells has been cultured ex vivo in a medium comprising the M1 peptide and/or IL-2.
A6a. The pharmaceutical combination of embodiment A5a, wherein the ex vivo culturing comprises:
   (i). culturing the population of cells comprising T cells ex vivo in a medium comprising the M1 peptide and IL-2;
   (ii). culturing the population of cells comprising T cells ex vivo in a medium comprising the M1 peptide, and then culturing the population of cells comprising T cells ex vivo in a medium comprising IL-2; or
   (iii). culturing the population of cells comprising T cells ex vivo in a medium comprising the M1 peptide, then culturing the population of cells comprising T cells ex vivo in a medium comprising the M1 peptide and IL-2; and then culturing the population of cells comprising T cells ex vivo in a medium comprising IL-2.
A7a. The pharmaceutical combination of embodiment A5a or A6a, wherein the population of cells comprising T cells has been cultured ex vivo for at least 1, 2, 3, 4, 5, 6, 7, 8, 9, 10, 11, 12, 13, or 14 days.
A8a. The pharmaceutical combination of any one of embodiments A2a to A7a, wherein the population of cells comprising T cells is a population of whole peripheral blood mononuclear cells (PBMCs).
A9a. The pharmaceutical combination of embodiment A8a, wherein the population of the whole PBMCs is from a healthy donor or an unhealthy donor.
A10a. The pharmaceutical combination of any one of embodiments A1a to A9a, wherein the Vβ17+CD8+ T cells have been isolated from the population of cells comprising T cells after contacting the population of cells comprising T cells with the M1 peptide and/or IL-2.
A11a. The pharmaceutical combination of any one of embodiments A1a to A10a, wherein the percent of Vβ17+CD8+ T cells in the isolated population of cells is more than 6%, 10%, 15%, 20%, 25%, 30%, 35%, 40%, 45%, 50%, 55%, 60%, 65%, 70%, 75%, 80%, 85%, or 90%.
A12a. The pharmaceutical combination of any one of embodiments A1a to A11a, wherein at least part of the Vβ17⁺CD8⁺ T cells express a cell surface receptor capable of binding to the M1 peptide.
A13a. The pharmaceutical combination of any one of embodiments A1a to A12a, wherein each of the T cell engagers is a multi-specific antibody.
A14a. The pharmaceutical combination of any one of embodiments A1a to A13a, wherein each of the T cell engagers comprises:
   1) a first binding domain that binds to an antigen expressed on a Vβ17+CD8+ T cell; and
   2) a second binding domain that binds to an antigen expressed on an unhealthy cell.
A15a. The pharmaceutical combination of embodiment A14a, wherein the antigen expressed on the Vβ17+CD8+ T cell is T Cell Receptor Beta Variable 19 (TRBV19) or Vβ17.
A15a-1. The pharmaceutical combination of embodiment A14, wherein the first binding domain comprises an antibody or an antigen-binding fragment thereof comprising: (i) a VH comprising a VH CDR1, a VH CDR2, and a VH CDR3 having an amino acid sequence of a VH CDR1, VH CDR2, and VH CDR3, respectively, of SEQ ID NO:16; and (ii) a VL comprising a VL CDR1, a VL CDR2, and a VL CDR3 having an amino acid sequence of a VL CDR1, VL CDR2, and VL CDR3, respectively, of SEQ ID NO:17.
A15a-2. The pharmaceutical combination of embodiment A15a-1, wherein the first binding domain comprises an antibody or an antigen-binding fragment thereof comprising: (i) a VH comprising a VH CDR1 having the amino acid sequence of SEQ ID NO:7, a VH CDR2 having the amino acid sequence of SEQ ID NO:8, and a VH CDR3 having the amino acid sequence of SEQ ID NO:9; and (ii) a VL comprising a VL CDR1 having the amino acid sequence of SEQ ID NO:10, a VL CDR2 having the amino acid sequence of SEQ ID NO:11, and a VL CDR3 having the amino acid sequence of SEQ ID NO:12.
A15a-3. The pharmaceutical combination of embodiment A14a, wherein the second binding domain comprises an antibody or an antigen-binding fragment thereof comprising: (i) a VH comprising a VH CDR1, a VH CDR2, and a VH CDR3 having an amino acid sequence of a VH CDR1, VH CDR2, and VH CDR3, respectively, of SEQ ID NO:40; and (ii) a VL comprising a VL CDR1, a VL CDR2, and a VL CDR3 having an amino acid sequence of a VL CDR1, VL CDR2, and VL CDR3, respectively, of SEQ ID NO:41.
A15a-4. The pharmaceutical combination of embodiment A15a-3, wherein the second binding domain comprises an antibody or an antigen-binding fragment thereof comprising: (i) a VH comprising a VH CDR1 having the amino acid sequence of SEQ ID NO:34, a VH CDR2 having the amino acid sequence of SEQ ID NO:35, and a VH CDR3 having the amino acid sequence of SEQ ID NO:36; and (ii) a VL comprising a VL CDR1 having the amino acid sequence of SEQ ID NO:37, a VL CDR2 having the amino acid sequence of SEQ ID NO:38, and a VL CDR3 having the amino acid sequence of SEQ ID NO:39.
A15a-5. The pharmaceutical combination of embodiment A14a, wherein the second binding domain comprises an antibody or an antigen-binding fragment thereof comprising: (i) a VH comprising a VH CDR1, a VH CDR2, and a VH CDR3 having an amino acid sequence of a VH CDR1, VH CDR2, and VH CDR3, respectively, of SEQ ID NO:78; and (ii) a VL comprising a VL CDR1, a VL CDR2, and a VL CDR3 having an amino acid sequence of a VL CDR1, VL CDR2, and VL CDR3, respectively, of SEQ ID NO:79.
A15a-6. The pharmaceutical combination of embodiment A15a-5, wherein the second binding domain comprises an antibody or an antigen-binding fragment thereof comprising: (i) a VH comprising a VH CDR1 having the amino acid sequence of SEQ ID NO:72, a VH CDR2 having the amino acid sequence of SEQ ID NO:73, and a VH CDR3 having the amino acid sequence of SEQ ID NO:74; and (ii) a VL comprising a VL CDR1 having the amino acid sequence of SEQ ID NO:75, a VL CDR2 having the amino acid sequence of SEQ ID NO:76, and a VL CDR3 having the amino acid sequence of SEQ ID NO:77.
A16a. The pharmaceutical combination of embodiment A14a or A15a, wherein the unhealthy cell is a cancer cell.
A17a. The pharmaceutical combination of embodiment A16a, wherein the cancer cell is a blood cancer cell or a solid tumor cancer cell.
A18a. The pharmaceutical combination of any one of embodiments A14a to A17a, and wherein the antigen expressed on the unhealthy cell is a tumor-associated antigen (TAA).
A19a. The pharmaceutical combination of embodiment A18a, wherein the TAA is CD123, ILIRAP, PSMA, or B7H3.
A20a. The pharmaceutical combination of any one of embodiments A1a to A19a, wherein each of the T cell engagers is a TRBV19xTAA or Vβ17xTAA bi-specific antibody.

In another set of embodiments (embodiment set B), provided are:
B1. A method for redirecting a T cell to a target cell, comprising contacting the target cell with one or more T cell engagers in the presence of an isolated population of cells comprising Vβ17+CD8+ T cells, wherein the contacting directs the Vβ17+CD8+ T cell to the target cell.
B2. The method of embodiment B1, wherein the isolated population of cells comprising Vβ17+CD8+ T cells has been activated or enriched via contacting a M1 peptide derived from human influenza A virus (M1₅₈₋₆₆) with a population of cells comprising T cells.
B3. The method of embodiment B2, wherein the M1 peptide comprises an amino acid sequence of GILGFVFTL (SEQ ID NO:1).
B4. The method of any one of embodiments B1 to B3, wherein the isolated population of cells comprising Vβ17+CD8+ T cells has been activated or enriched via further contacting IL-2 with the population of cells comprising T cells.
B5. The method of any one of embodiments B2 to B4, wherein the population of cells comprising T cells has been cultured ex vivo in a medium comprising the M1 peptide and/or IL-2.
B6. The method of embodiment B5, wherein the ex vivo culturing comprises:
   (i). culturing the population of cells comprising T cells ex vivo in a medium comprising the M1 peptide and IL-2;
   (ii). culturing the population of cells comprising T cells ex vivo in a medium comprising the M1 peptide, and then culturing the population of cells comprising T cells ex vivo in a medium comprising IL-2; or
   (iii). culturing the population of cells comprising T cells ex vivo in a medium comprising the M1 peptide, then culturing the population of cells comprising T cells ex vivo in a medium comprising the M1 peptide and IL-2; and then culturing the population of cells comprising T cells ex vivo in a medium comprising IL-2.
B7. The method of embodiment B5 or B6, wherein the population of cells comprising T cells has been cultured ex vivo for at least 1, 2, 3, 4, 5, 6, 7, 8, 9, 10, 11, 12, 13, or 14 days.
B8. The method of any one of embodiments B2 to B7, wherein the population of cells comprising T cells is a population of whole peripheral blood mononuclear cells (PBMCs).
B9. The method of embodiment B8, wherein the population of the whole PBMCs is from a healthy donor or an unhealthy donor.
B10. The method of any one of embodiments B1 to B9, wherein the Vβ17+CD8+ T cells have been isolated from the population of cells comprising T cells after contacting the population of cells comprising T cells with the M1 peptide and/or IL-2.
B11. The method of any one of embodiments B1 to B10, wherein the percent of Vβ17+CD8+ T cells in the isolated population of cells is more than 6%, 10%, 15%, 20%, 25%, 30%, 35%, 40%, 45%, 50%, 55%, 60%, 65%, 70%, 75%, 80%, 85%, or 90%.
B12. The method of any one of embodiments B1 to B11, wherein at least part of the Vβ17⁺CD8⁺ T cells express a cell surface receptor capable of binding to the M1 peptide.
B13. The method of any one of embodiments B1 to B12, wherein each of the T cell engagers is a multi-specific antibody.
B14. The method of any one of embodiments B1 to B13, wherein each of the T cell engagers comprises:
   1) a first binding domain that binds to an antigen expressed on a Vβ17+CD8+ T cell; and
   2) a second binding domain that binds to an antigen expressed on an unhealthy cell.
B15. The method of embodiment B14, wherein the antigen expressed on the Vβ17+CD8+ T cell is T Cell Receptor Beta Variable 19 (TRBV19) or Vβ17.
B16. The method of embodiment B14 or B15, wherein the unhealthy cell is a cancer cell.
B17. The method of embodiment B16, wherein the cancer cell is a blood cancer cell or a solid tumor cancer cell.
B18. The method of any one of embodiments B14 to B17, and wherein the antigen expressed on the unhealthy cell is a tumor-associated antigen (TAA).
B19. The method of embodiment B18, wherein the TAA is CD123, ILIRAP, PSMA, or B7H3.
B20. The method of any one of embodiments B1 to B19, wherein each of the T cell engagers is a TRBV19xTAA or Vβ17xTAA bi-specific antibody.

In another set of embodiments (embodiment set C), provided are:
C1. A method for inhibiting the growth or proliferation of a target cell, comprising contacting the target cell with one or more T cell engagers in the presence of an isolated population of cells comprising Vβ17+CD8+ T cells, wherein the contacting results in the inhibition of the growth or proliferation of the target cell.
C2. The method of embodiment C1, wherein the isolated population of cells comprising Vβ17+CD8+ T cells has been activated or enriched via contacting a M1 peptide derived from human influenza A virus (M1₅₈₋₆₆) with a population of cells comprising T cells.
C3. The method of embodiment C2, wherein the M1 peptide comprises an amino acid sequence of GILGFVFTL (SEQ ID NO:1).
C4. The method of any one of embodiments C1 to C3, wherein the isolated population of cells comprising Vβ17+CD8+ T cells has been activated or enriched via further contacting IL-2 with the population of cells comprising T cells.
C5. The method of any one of embodiments C2 to C4, wherein the population of cells comprising T cells has been cultured ex vivo in a medium comprising the M1 peptide and/or IL-2.
C6. The method of embodiment C5, wherein the ex vivo culturing comprises:
   (i). culturing the population of cells comprising T cells ex vivo in a medium comprising the M1 peptide and IL-2;
   (ii). culturing the population of cells comprising T cells ex vivo in a medium comprising the M1 peptide, and then culturing the population of cells comprising T cells ex vivo in a medium comprising IL-2; or
   (iii). culturing the population of cells comprising T cells ex vivo in a medium comprising the M1 peptide, then culturing the population of cells comprising T cells ex vivo in a medium comprising the M1 peptide and IL-2; and then culturing the population of cells comprising T cells ex vivo in a medium comprising IL-2.
C7. The method of embodiment C5 or C6, wherein the population of cells comprising T cells has been cultured ex vivo for at least 1, 2, 3, 4, 5, 6, 7, 8, 9, 10, 11, 12, 13, or 14 days.
C8. The method of any one of embodiments C2 to C7, wherein the population of cells comprising T cells is a population of whole peripheral blood mononuclear cells (PBMCs).
C9. The method of embodiment C8, wherein the population of the whole PBMCs is from a healthy donor or an unhealthy donor.
C10. The method of any one of embodiments C1 to C9, wherein the Vβ17+CD8⁺ T cells have been isolated from the population of cells comprising T cells after contacting the population of cells comprising T cells with the M1 peptide and/or IL-2.
C11. The method of any one of embodiments C1 to C10, wherein the percent of Vβ17+CD8+ T cells in the isolated population of cells is more than 6%, 10%, 15%, 20%, 25%, 30%, 35%, 40%, 45%, 50%, 55%, 60%, 65%, 70%, 75%, 80%, 85%, or 90%.
C12. The method of any one of embodiments C1 to C11, wherein at least part of the Vβ17⁺CD8⁺ T cells express a cell surface receptor capable of binding to the M1 peptide.
C13. The method of any one of embodiments C1 to C12, wherein each of the T cell engagers is a multi-specific antibody.
C14. The method of any one of embodiments C1 to C13, wherein each of the T cell engagers comprises:
   1) a first binding domain that binds to an antigen expressed on a Vβ17+CD8+ T cell; and
   2) a second binding domain that binds to an antigen expressed on an unhealthy cell.
C15. The method of embodiment C14, wherein the antigen expressed on the Vβ17+CD8+ T cell is T Cell Receptor Beta Variable 19 (TRBV19) or Vβ17.
C16. The method of embodiment C14 or C15, wherein the unhealthy cell is a cancer cell.
C17. The method of embodiment C16, wherein the cancer cell is a blood cancer cell or a solid tumor cancer cell.
C18. The method of any one of embodiments C14 to C17, and wherein the antigen expressed on the unhealthy cell is a tumor-associated antigen (TAA).
C19. The method of embodiment C18, wherein the TAA is CD123, ILIRAP, PSMA, or B7H3.
C20. The method of any one of embodiments C1 to C19, wherein each of the T cell engagers is a TRBV19xTAA or Vβ17xTAA bi-specific antibody.

In yet another set of embodiments (embodiment set D), provided are:
D1. A method for eliminating a target cell, comprising contacting the target cell with one or more T cell engagers in the presence of an isolated population of cells comprising Vβ17+CD8+ T cells, wherein the contacting results in the elimination of the target cell.
D2. The method of embodiment D1, wherein the isolated population of cells comprising Vβ17+CD8+ T cells has been activated or enriched via contacting a M1 peptide derived from human influenza A virus (M1₅₈₋₆₆) with a population of cells comprising T cells.
D3. The method of embodiment D2, wherein the M1 peptide comprises an amino acid sequence of GILGFVFTL (SEQ ID NO:1).
D4. The method of any one of embodiments D1 to D3, wherein the isolated population of cells comprising Vβ17+CD8+ T cells has been activated or enriched via further contacting IL-2 with the population of cells comprising T cells.
D5. The method of any one of embodiments D2 to D4, wherein the population of cells comprising T cells has been cultured ex vivo in a medium comprising the M1 peptide and/or IL-2.
D6. The method of embodiment D5, wherein the ex vivo culturing comprises:
   (i). culturing the population of cells comprising T cells ex vivo in a medium comprising the M1 peptide and IL-2;
   (ii). culturing the population of cells comprising T cells ex vivo in a medium comprising the M1 peptide, and then culturing the population of cells comprising T cells ex vivo in a medium comprising IL-2; or
   (iii). culturing the population of cells comprising T cells ex vivo in a medium comprising the M1 peptide, then culturing the population of cells comprising T cells ex vivo in a medium comprising the M1 peptide and IL-2; and then culturing the population of cells comprising T cells ex vivo in a medium comprising IL-2.
D7. The method of embodiment D5 or D6, wherein the population of cells comprising T cells has been cultured ex vivo for at least 1, 2, 3, 4, 5, 6, 7, 8, 9, 10, 11, 12, 13, or 14 days.
D8. The method of any one of embodiments D2 to D7, wherein the population of cells comprising T cells is a population of whole peripheral blood mononuclear cells (PBMCs).
D9. The method of embodiment D8, wherein the population of the whole PBMCs is from a healthy donor or an unhealthy donor.
D10. The method of any one of embodiments D1 to D9, wherein the Vβ17+CD8+ T cells have been isolated from the population of cells comprising T cells after contacting the population of cells comprising T cells with the M1 peptide and/or IL-2.
D11. The method of any one of embodiments D1 to D10, wherein the percent of Vβ17+CD8+ T cells in the isolated population of cells is more than 6%, 10%, 15%, 20%, 25%, 30%, 35%, 40%, 45%, 50%, 55%, 60%, 65%, 70%, 75%, 80%, 85%, or 90%.
D12. The method of any one of embodiments D1 to D11, wherein at least part of the Vβ17⁺CD8⁺ T cells express a cell surface receptor capable of binding to the M1 peptide.
D13. The method of any one of embodiments D1 to D12, wherein each of the T cell engagers is a multi-specific antibody.
D14. The method of any one of embodiments D1 to D13, wherein each of the T cell engagers comprises:
   1) a first binding domain that binds to an antigen expressed on a Vβ17+CD8+ T cell; and
   2) a second binding domain that binds to an antigen expressed on an unhealthy cell. D15. The method of embodiment D14, wherein the antigen expressed on the Vβ17+CD8+ T cell is T Cell Receptor Beta Variable 19 (TRBV19) or Vβ17.
D16. The method of embodiment D14 or D15, wherein the unhealthy cell is a cancer cell.
D17. The method of embodiment D16, wherein the cancer cell is a blood cancer cell or a solid tumor cancer cell.
D18. The method of any one of embodiments D14 to D17, and wherein the antigen expressed on the unhealthy cell is a tumor-associated antigen (TAA).
D19. The method of embodiment D18, wherein the TAA is CD123, ILIRAP, PSMA, or B7H3.
D20. The method of any one of embodiments D1 to D19, wherein each of the T cell engagers is a TRBV19xTAA or Vβ17xTAA bi-specific antibody.

In yet another set of embodiments (embodiment set E), provided are:
E1. A method for treating a disease or disorder in a subject comprising administering to the subject: (i) a therapeutically effective amount of an isolated population of cells comprising Vβ17+CD8+ T cells, and (ii) a therapeutically effective amount of one or more T cell engagers.
E2. The method of embodiment E1, wherein the isolated population of cells comprising Vβ17+CD8+ T cells has been activated or enriched via contacting a M1 peptide derived from human influenza A virus (M1₅₈₋₆₆) with a population of cells comprising T cells.
E3. The method of embodiment E2, wherein the M1 peptide comprises the amino acid sequence of GILGFVFTL (SEQ ID NO:1).
E4. The method of any one of embodiments E1 to E3, wherein the isolated population of cells comprising Vβ17⁺CD8+ T cells has been activated or enriched via further contacting IL-2 with the population of cells comprising T cells.
E5. The method of any one of embodiments E2 to E4, wherein the population of cells comprising T cells has been cultured ex vivo in a medium comprising the M1 peptide and/or IL-2.
E6. The method of embodiment E5, wherein the ex vivo culturing comprises:
   (i). culturing the population of cells comprising T cells ex vivo in a medium comprising the M1 peptide and IL-2;
   (ii). culturing the population of cells comprising T cells ex vivo in a medium comprising the M1 peptide, and then culturing the population of cells comprising T cells ex vivo in a medium comprising IL-2; or
   (iii). culturing the population of cells comprising T cells ex vivo in a medium comprising the M1 peptide, then culturing the population of cells comprising T cells ex vivo in a medium comprising the M1 peptide and IL-2; and then culturing the population of cells comprising T cells ex vivo in a medium comprising IL-2.
E7. The method of embodiment E5 or E6, wherein the population of cells comprising T cells has been cultured ex vivo for at least 1, 2, 3, 4, 5, 6, 7, 8, 9, 10, 11, 12, 13, or 14 days.
E8. The method of any one of embodiments E2 to E7, wherein the population of cells comprising T cells is a population of whole peripheral blood mononuclear cells (PBMCs).
E9. The method of embodiment E8, wherein the population of the whole PBMCs is from a healthy donor or an unhealthy donor.
E10. The method of any one of embodiments E1 to E9, wherein the Vβ17+CD8+ T cells have been isolated from the population of cells comprising T cells after contacting the population of cells comprising T cells with the M1 peptide and/or IL-2.
E11. The method of any one of embodiments E1 to E10, wherein the percent of Vβ17+CD8+ T cells in the isolated population of cells is more than 6%, 10%, 15%, 20%, 25%, 30%, 35%, 40%, 45%, 50%, 55%, 60%, 65%, 70%, 75%, 80%, 85%, or 90%.
E12. The method of any one of embodiments E1 to E11, wherein at least part of the Vβ17⁺CD8⁺ T cells express a cell surface receptor capable of binding to the M1 peptide.
E13. The method of any one of embodiments E1 to E12, wherein each of the T cell engagers is a multi-specific antibody.
E14. The method of any one of embodiments E1 to E13, wherein each of the T cell engagers comprises:
   1) a first binding domain that binds to an antigen expressed on a Vβ17+CD8+ T cell; and
   2) a second binding domain that binds to an antigen expressed on an unhealthy cell.
E15. The method of embodiment E14, wherein the antigen expressed on the Vβ17+CD8+ T cell is T Cell Receptor Beta Variable 19 (TRBV19) or Vβ17.
E16. The method of embodiment E14 or E15, wherein the unhealthy cell is a cancer cell.
E17. The method of embodiment E16, wherein the cancer cell is a blood cancer cell or a solid tumor cancer cell.
E18. The method of any one of embodiments E14 to E17, and wherein the antigen expressed on the unhealthy cell is a tumor-associated antigen (TAA).
E19. The method of embodiment E18, wherein the TAA is CD123, ILIRAP, PSMA, or B7H3.
E20. The method of any one of embodiments E1 to E19, wherein each of the T cell engagers is a TRBV19xTAA or Vβ17xTAA bi-specific antibody.
E21. The method of any one of embodiments E1 to E20, wherein the disease or disorder is cancer.
E22. The method of embodiment E21, wherein the cancer is a blood cancer.
E23. The method of embodiment E21, wherein the cancer is a solid tumor cancer.
E24. The method of any one of embodiments E1 to E23, wherein the subject is a human subject in need thereof.

### 7. EXAMPLES

The following is a description of various methods and materials used in the studies, and are put forth so as to provide those of ordinary skill in the art with a complete disclosure and description of how to make and use the present disclosure, and are not intended to limit the scope of what the inventors regard as their disclosure nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present disclosure. Efforts have been made to ensure accuracy with respect to numbers used (e.g., amounts, percentages, etc.), but some experimental errors and deviations should be accounted for.

### 7.1. Example 1—Materials and Methods

### 7.1.1. Preparation of Bispecific Antibodies

The variable region sequence of anti-TRBV19 and anti-TAA (Tumor Associated Antigen) was used to generate a bispecific antibody to be tested for T cell re-directed killing of acute myeloid leukemia (AML) cells. The bispecific antibodies were produced as full-length antibodies in the knob-into-hole format as human IgG1. Nucleic acid sequences encoding variable regions were sub-cloned into a custom mammalian expression vectors containing constant region of IgG1 expression cassettes using standard PCR restriction enzyme based standard cloning techniques, and sequenced verified. The bispecific antibodies were expressed by transient transfection in Chinese hamster ovary cell line. The antibodies were initially purified by Mab Select SuRe Protein A column (GE Healthcare). The column was equilibrated with PBS pH 7.2 and loaded with fermentation supernatant at a flow rate of 2 mL/min. After loading, the column was washed with 4 column volumes of PBS followed by elution in 30 mM sodium acetate, pH 3.5. Fractions containing protein peaks as monitored by absorbance at 280 nm were pooled and neutralized to pH 5.0 by adding 1% 3 M sodium acetate pH 9.0. The bispecific mAbs were further purified on a preparative Superdex 200 10/300 GL (GE healthcare) size exclusion chromatography (SEC) column equilibrated with PBS buffer. The integrity of sample was assessed by endotoxin measurement and SDS-PAGE under reducing and non-reducing conditions. The final protein concentrations were 1.0 mg/ml and the final endotoxin levels were <3.0 EU/mg.

### 7.1.2. Production of Recombinant Antigen (Vβ17-Vα10.2-Fc) Protein

The extracellular domain of heterodimer Vβ17-Vα10.2 TCR containing a C-terminal human IgG1 Fc tag was expressed as a secreted protein in ExpiCHO cell line as described previously. Purification protocol identical to the mAbs, except that the final protein was dialyzed into PBS pH 6.8. Purity was determined by SDS-PAGE and SEC to 99.5%.

### 7.1.3. HDX Epitope Mapping of 7A5 mAb on TCR Vg9 Protein

The procedures used to analyze the mAb perturbation were carried out as previously described with minor modifications. Recombinant human Vγ9-Vδ2 was incubated with and without anti-Vγ9 7A5 mAb (in BSA-free PBS pH 7.2 buffer) in 118 μL deuterium oxide labeling buffer (50 mM sodium phosphate, 100 mM sodium chloride at pD 7.4) at 10° C. At time points 0 sec, 10 sec, 60 sec, 300 sec, 1800 sec or 7200 sec, hydrogen-deuterium exchange (HDX) mixture was quenched by adding 130 μL of 4 M guanidine hydrochloride, 0.85 M TCEP buffer followed by a 3 min incubation at 10° C. Final pH is ˜2.5. The quenched samples were subjected to online pepsin/protease XIII digestion using an in-house packed pepsin/protease XIII column (2.1×30 mm). The resultant peptides were analyzed using an UPLC-MS system comprised of a Waters Acquity UPLC coupled to a Q Exactive™ Hybrid Quadrupole-Orbitrap Mass Spectrometer (Thermo). The peptides were separated on a 50×1 mm C8 column with a 16.5 min gradient from 2-31% solvent B (0.2% formic acid in acetonitrile). Solvent A was 0.2% formic acid in water. The injection valve and pepsin/protease XIII column and their related connecting tubings were inside a cooling box maintained at 15° C. for native protein. The second switching valve, C8 column and their related connecting stainless-steel tubings were inside another chilled circulating box maintained at −6° C. Peptide identification was done through searching MS/MS data against the Vγ9-Vδ2 TCR sequence with Mascot. The mass tolerance for the precursor and product ions were 7 ppm and 0.02 Da, respectively. Raw MS data was processed using HDX WorkBench, software for the analysis of HDX MS data. The deuterium levels were calculated using the average mass difference between the deuterated peptide and its native form (t0).

### 7.1.4. Binding Kinetics for Anti-V79 mAb to TCR V79-V62 by SPR

Data was obtained using ProteOn XPR36 Surface Plasmon Resonance (SPR) System from BioRad. The experiments were carried out in HBSP buffer at 25° C. The experimental set up was as the following: Goat anti-murine Fc was immobilized on the surface of a GLC chip, and binding was tested by capturing the mouse anti-human TCR Vg9 [clone 7A5] mAb at different densities. The construct of monovalent Vg9-Vd2 heterodimer fused to human Fc in solution at 0.3 μM in 3-fold dilution series were flowed through anti-TCR Vg9 mAb that was captured on the surface of the GLC chip. Association and dissociation times was set to 4 min and 30 min, respectively. Raw binding data were processed by double referencing: 1) interspot on an empty chip surface; 2) column 6 where no 7A5 was captured, to monitor the noise to background of the antigen binding to the GAM-Fc capture surface. Data was global fitted to a 1:1 simple Langmuir binding model.

### 7.1.5. Cell Lines and Reagents

Cell lines (Kasumi-3; acute myeloblastic leukemia cell line, 22Rv1; human prostate carcinoma cell line, and H1975; human lung adenocarcinoma cell line) used in this study were purchased from ATCC. Kasumi-3 cells were cultured in RPMI-1640-1640+20% FBS+1×Pen/Strep. 22Rv1 and H1975 cell lines were cultured in RPMI-16401640+10% FBS+1×Pen/Strep medium. Each cell line was cultured according to the culture method specified by its supplier. Five to ten vials of cells from initial passages were frozen as stocks, from which cells with fewer than 20 passages were used for all the experiments. All cell culture media and supplements were purchased from Gibco (Thermo Fisher Scientific Inc, Waltham, MA). CFSE (Carboxyfluorescein succinimidyl ester) and 7-AAD (7-Aminoactinomycin D) reagents were obtained from Thermo scientific and BioLegend respectively.

### 7.1.6. PBMC Isolation

Peripheral Blood Mononuclear Cells (PBMCs) were isolated from healthy donor's blood as described elsewhere. Briefly, whole blood was diluted in plain RPMI-1640 medium at 1:1 ratio and carefully layered onto Lymphoprep™ gradient (STEMCELL Technologies, Vancouver, Canada) in a 50 mL falcon Tube (Corning, NY, USA). Centrifuged the tube at 450×g for 30 min at room temperature with acceleration and deacceleration was kept at 0. After centrifugation, cells were collected from the interface and erythrocyte lysis was performed using erythrocyte lysis buffer (Sigma, St. Louis, MO) for 5 min at room temperature. Supernatant containing lysed erythrocyte was discarded and the cell pellet was washed twice with plain RPMI-1640 medium. After washes, cells were resuspended in culture medium (RPMI-1640+10% FBS+1% Pens/Strep), counted and used it for downstream applications or frozen down in the freezing medium (90% FBS+10% DMSO) at a density of 25×106 cells/mL and stored in liquid nitrogen until further use.

### 7.1.7. Flow Cytometry

All flow cytometry studies were carried out on Novocyte flow cytometer (ACEA biosciences, Singapore) and data was analyzed by Flowjo analysis software (Treestar Inc, Ashland, OR). All antibodies used in this study were purchased from BioLegend unless and until specified. Anti-human Vβ17 antibody was procured from Beckman Coulter. Please refer to the supplemental materials methods for the entire list of the antibodies used in this study (Table 1). For surface staining, cells were initially Fc blocked with human TruStain FcX (Biolegend, San Diego, CA) in the culture medium (RPMI-1640+10% FBS+1×Pen/Str) for 20 minutes at 4° C. Washed once with wash buffer (PBS+2% FBS) and stained with LIVE/DEAD™ Fixable violet dye (Thermo Fischer Scientific Inc, Waltham, CA) in PBS for 20 minutes at room temperature. Alternatively, cells were incubated in PBS containing human TruStain FcX and LIVE/DEAD™ Fixable violet dye at 4° C. for 30 minutes. After two washes, cells were surface stained with antibodies specific for cell surface antigens for 30 minutes at 4° C. After incubation period, cells were washed twice and acquired on Novocyte flow cytometer immediately. Alternatively, cells were fixed with 4% Paraformaldehyde (PFA) for 30 minutes at 4° C., washed twice with wash buffer, resuspended in wash buffer (PBS+2% FBS) and acquired on flow cytometer within 48 hours of fixation.

7.1.8. Expansion of Vβ17+ Cell from Whole PBMCs

Vβ17+ T cells were expanded from whole PBMCs using a flu specific M1 peptide (flu MP58 peptide (GILGFVFTL), New England Peptide company, MA, USA). Briefly, on day 0, 2.5×106 PBMCs were seeded into a well of 6-well plate containing 2.5 ml of culture medium (RPMI-1640-1640+10% FBS+1×Pen/Strep) containing 1 μM of M1 peptide and 210 IU IL-2 (R&D Systems, Minneapolis, USA). On day 2, 4, 8, 10 and 12 of the culture, IL-2 was replenished to the culture at a final concentration of 201 IU/mL. On day 14 of the culture, PBMCs were harvested and determined the frequency of Vβ17+ cells among Pan-T cells or CD8+ T cells by flow cytometry after staining them with anti-human CD3, CD8 and Vβ17 antibodies

### 7.1.9. T Cell Enrichment

Pan-T cells or CD8+ T cells were isolated either from fresh PBMCs or PBMCs cultured with M1 peptide+IL-2 for 14 days, using EasySep™ human T cell isolation kit or EasySep™ human CD8+ T cell isolation kit (Stem cell Technologies, Vancouver, Canada) respectively, as per manufacturer's instructions. Purity of enriched Pan-T cells or CD8+ T cells was assessed by flow cytometry after staining them with anti-CD3 and anti-CD8 antibodies respectively.

### 7.1.10. Bispecific Antibody Binding Assay

Binding of Vβ17 bispecific antibodies to its respective tumor associated antigen (TAA) expressing cell line and CD8+ T cells was carried out by flow cytometry. Briefly, 50,000 target cells or CD8+ T cells were incubated at 4° C. for 45 minutes with serial dilutions of various bispecific antibodies or its null arm control bispecific antibodies. After washing with wash buffer (PBS+2% FBS), bispecific antibody bound to cell surface was detected by incubating the cells with PE labelled mouse anti human IgG4 secondary antibody (SouthernBiotech, Birmingham, AL) for 30 minutes at 4° C. Cells were washed with wash buffer (PBS+2% FBS) and the fluorescence of stained cells was measured on Novocyte flow cytometer. Cells were visualized on forward and sideward scatter and doublets were excluded. No secondary antibody control was used to establish background fluorescence and to gate on specific population. Background value was subtracted from main samples to get specific binding value. Since Vβ17+ cell population among CD8+ T cells was around 30% (in case of PBMCs stimulated with M1peptide+IL-2 for 14 days) or around 5-6% (in case of fresh PBMCs), maximum binding obtained at highest concentration of bispecific antibody was assumed as 100% and extrapolated all the values at other concentrations of bispecific antibody accordingly. For all binding assays, tumor associated antigen (TAA) expression on cell lines and the abundance of Vβ17+ cells among Pan-T cells or CD8+ T cells were assessed by staining them with specific monoclonal antibody.

### 7.1.11. Depletion of Immune Cell Subsets

Depletion of specific cell subsets was achieved by FACS-sorting. For flow cytometry based cell sorting, CD8+ T cells and Pan-T cells were enriched using EasySep™ human CD8+ T cell isolation kit or EasySep™ human T cell isolation kit (Stem cell Technologies) respectively, as per the manufacturer's instructions. Enriched cells were washed twice with wash buffer and stained with anti-human Vβ17 antibody for 30 minutes at 4° C. Stained cells were FACS sorted as Vβ17−CD8+ T cells on FACS ARIA (BD biosciences, San Jose, CA). Sorted Vβ17−CD8+ T cells purity was assessed via flow cytometry.

### 7.1.12. In Vitro Cytotoxicity Assay

Efficacy of Vβ17 bispecific antibody in mediating tumor cell lysis was assessed by flow cytometry-based cytotoxicity assay. In brief, target cells were labelled with CFSE by incubating the cells in 0.5 uM CFSE (Thermo Fischer Scientific Inc, Waltham, CA) at room temperature for 8 min. After the staining period, labelling was stopped by adding 1 mL of FBS (Gibco). After washing twice with culture medium, cells were counted and resuspended in complete medium. Effector cells (Pan-T cells or CD8+ T cells) were enriched from whole PBMCs or PBMCs cultured with M1 peptide+IL-2 for 14 days by using Pan-T cell or CD8+ T cell isolation kit, as per manufacturer's instructions. After enrichment, cells were counted and resuspended in culture medium. CFSE labelled target cells were seeded either on the day of the co-culture (for suspension target cell lines) or seeded as a monolayer (for adherent cell lines) a day before the onset of co-culture. Effector and target cells were co-cultured at various effector to target (E:T) ratios in the presence or absence of bispecific antibodies at 37° C., 5% CO₂ for various time points. ET ratio was normalized to Vβ17+ cells in all the experiments concerned to Vβ17 bispecific mediated T cell cytotoxicity evaluation. For assessing cytotoxicity at the end of the incubation period, 7-AAD was added to the effector-target co-culture and acquired cells on Novocyte flow cytometer. For assessing cytotoxicity of adherent target cell lines (22Rv1 and H1975), adherent cells were detached using trypsin-EDTA (Gibco) and 7-AAD was added to the detached target cells. To identify target cell death, CFSE positive cells were initially gated on to identify the target cells. Within the CFSE positive target cells, dead cells were identified as 7-AAD+ FSClow cells. Gates were set based on the CFSE unstained and 7-AAD unstained cells. To calculate bispecific antibody mediated specific killing, cell lysis value from no bispecific antibody control well was subtracted out from total cell death value from the wells containing indicated bispecific antibodies. Spontaneous cytotoxicity of target cells was assessed by culturing them without effector cells or bispecific antibodies.

### 7.1.13. Cytokine and Effector Molecule Analysis

Cytokines and effector molecules were assessed both intracellularly and in the cell culture supernatants. For intracellular cytokine and effector molecules assessment by flow cytometry, cells were initially surface stained with indicated monoclonal antibodies and washed twice with wash buffer. Stained cells were fixed and permeabilized using BD fix/Perm kit (BD biosciences) as per manufacturer's instructions. Permeabilized cells were probed with monoclonal antibodies against intracellular cytokines (TNFα, IFNγ) or effector molecules (Granzyme B, Perforin) for 30 minutes at 4° C. Cells were washed twice and acquired on Novocyte flow cytometer. FMO (Fluorescence Minus One) controls were used to establish the gating for cytokines. For assessing cytokines in cell culture supernatant, cell culture supernatants were collected on indicated time points and were subjected to quantification using customized human magnetic luminex assay 15 plex kit (R& D systems, Minneapolis, USA), as per the manufacturer's instructions. Quantification of the cytokines was carried out in MagPix multiplex detection system with xPONENT software.

### 7.1.14. Whole PBMC Assay

Whole PBMC assay was carried out to assess the efficacy of bispecific antibody in mediating activation, proliferation, differentiation and effector profile of Vβ17+ T cells. Briefly, CFSE labelled whole PBMCs (0.1×106 cells in 200 μL of culture medium) were cultured in the presence of either Vβ17XCD123 or Vβ17XNULL or CD3XCD123 or CD3XNULL bispecific antibodies at a concentration of 5 ng/mL. As a control, CFSE labelled whole PBMCs were cultured without any bispecific antibodies. On day 3 of PBMCs culture, Vβ17+ and Vβ17− cells among CD8+ or CD4+ T cells were assessed for their activation, by staining them with antibodies specific for cell surface CD25 and CD71 markers, and differentiation status, by probing for intracellular Granzyme B and Perforin, from culture plate wells that contains bispecific antibody, compared to no bispecific antibody wells. On day 5 of PBMCs culture, bispecific antibody mediated T cell cytotoxicity, as measured by the elimination of CD123+ cells, was assessed by staining the whole PBMCs with antibodies against CD123 and/or HLA-DR, CD14. On day 5 of PBMCs culture, cell culture supernatant was also collected for assessing the cytokines in cell culture supernatant. Similarly, whole PBMCs from AML patients were cultured in the presence of 50 ng/mL Vβ17/CD123 or Vβ17/Null BiAbs for 14 days with IL-2 (210 IU) addition on days 0, 5, 9, and 12, during the culture. Abundance of Vβ17+ cells among CD8 and CD4 T cells was assessed on day 9 and day 8 of the culture period. Further, on day 14, CD8⁺ T cells (effectors) were enriched and co-cultured with Kasumi-3/22Rv1 cell line (targets) in the presence of 50 ng/mL Vβ17/CD123 and Vβ17/PSMA BiAb for 24 (Kasumi-3) and 72 (22Rv1) hrs and assessed target cell lysis. Likewise, NHL PBMCs were cultured in the absence or presence of 1 μg/mL Vβ17/Dll3 BiAb with addition of IL-2 once in 2 days beginning from day 0. On day 7, abundance of Vβ17+ cells among CD8 T cells was assessed.

The null half antibody of the bispecific antibodies provided herein comprises the amino acid sequence of SEQ ID NO:83.

(B23B49)
SEQ ID NO: 83
DIVMTQSPDSLAVSLGERATINCRASQSVDYNGISYMHWYQQKPGQPPKL
LIYAASNPESGVPDRFSGSGSGTDFTLTISSLQAEDVAVYYCQQIIEDPW
TFGQGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGECGGSEGKSSGSGSESKSTEGKSSGSGSESKSTG
GSQITLKESGPTLVKPTQTLTLTCTFSGFSLSTSGMGVSWIRQPPGKALE
WLAHIYWDDDKRYNPSLKSRLTITKDTSKNQVVLTMTNMDPVDTATYYCA
RLYGFTYGFAYWGQGTLVTVSSASTKGPSVFPLAPCSRSTSESTAALGCL
VKDYFPEPVTVSWNSGALTSGVHTFPAVLQSSGLYSLSSVVTVPSSSLGT
KTYTCNVDHKPSNTKVDKRVESKYGPPCPPCPAPEAAGGPSVFLFPPKPK
DTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQV
YTLPPSQEEMTKNQVSLWCLVKGFYPSDIAVEWESNGQPENNYKTTPPVL
DSDGSFFLYSRLTVDKSRWQEGNVFSCSVMHEALHNHYTQKSLSLSLGK

### 7.1.15. Xenograft Tumor Model

All animal experiments were performed in strict accordance with rules approved by an in-house animal committee (IAEC). Six to eight weeks old NSG NOD.Cg-Prkdc<scid>Il2rg<tm1Wjl>/SzJ mice were purchased form The Jackson laboratories. The animals were quarantined for 1 week, followed by 1 week for acclimatization at in-house animal facility before starting of the experiment. All mice received a single s.c. injection of 5×106 KG-1 cells, mixed with 1:1 ratio of Matrigel, in the right flank. Seven days post s.c injection, mice were randomized based on the tumor size (˜50-70 mm3) and segregated into five groups of five mice each. After the randomization (Day 1), twenty million enriched Pan-T cells were intravenously administered to each mouse, followed by an i.v injection of 10 μg of bispecific antibody. Bispecific antibody treatment was repeated on day 3, 5, 8 and 10 of the experiment. As a control group, mice were either injected tumor alone or mice were injected with tumor plus pan-T cells without bispecific injection. Body weight and tumor volume were measured once every three days. Tumor volume (TV) was determined by measurements in two dimensions using digital Vernier calipers. Tumor volume (TV) was calculated using the following formula: Tumor Volume (mm3)=L×W/2. L=length (mm), W=width (mm). Mice were euthanized if the mean tumor volume is >1500 mm3 or the drop-in body weight loss is >20% (whichever is earlier). Post euthanasia, tumor was embedded in OCT and cryopreserved until further use. In parallel, enriched pan-T cells were labelled with 5 μM Dil dye (Molecular Probes, Invitrogen) at room temperature for 20 minutes. After two washes, ten million Dil dye labelled Pan T cells were injected intravenously into mice bearing subcutaneous KG-1 tumors. In vivo imaging (Burker, Fluorescence Modality Ex 550 nm; Em 600 nm; 15 sec exposure; Co-registered with X-ray 1.2 sec exposure) was carried out on mice that received Dil dye labelled Pan-T cells at 0, 1, 3, 6, 10, 24, 48, 72 and 96 hrs. From each group, one animal was euthanized after 24h, while remaining animals were imaged up to 96h. Tumor was harvested and fixed in OCT for cryo sectioning.

### 7.1.16. Sequential Killing Assessment

Whole CD8⁺ T cells/Pan-T cells or CD8⁺ T cells/Pan-T cells depleted of Vβ17⁺ cells (Effectors) were co-cultured with CFSE labelled target cells (Kasumi-3) in the presence of 5 ng/mL Vβ17/CD123 and CD3/CD123 BiAbs respectively for 3 days at an ET ratio of 1:1. For Vβ17/CD123 mediated cytotoxicity assessment, Vβ17+CD8+ T cells (Effectors) was normalized to target cells. After 3 days, co-cultured cells were washed extensively to get rid of the BiAb and determined the frequency of Vβ17⁺ (for Vβ17/CD123) or CD8⁺/CD3+ (for CD3/CD123) cells in co-culture setup. Washed effectors were re-challenged with CTV labelled Kasumi-3 cells at an ET ratio of 1:1 for 3 days (without any BiAb). At the end of the incubation period, co-cultured cells were washed, re-challenged them with fresh Kasumi-3 (CFSE labelled) cells after determining the frequency of Vβ17+ or CD8+/CD3+ T cells. Effector cells were subjected to several rounds of re-challenges with fresh targets every time and assessed the cytotoxicity (and the abundance of Vβ17+ cells among CD8/CD3+) on day 3 +5 +7 +10 +12 +14 +17 +19. On day 3+5, total co-cultured effector cells were profiled for various inhibitory receptor surface expression.

### 7.1.17. Assessing Effector Functions Under Normoxia/Hypoxia Conditions

Whole CD8+ T cells were co-cultured with target (Kasumi-3) cells at an ET ratio of 1:1 for 72 hrs in the absence or presence of 50 ng/mL of Vβ17/CD123 or Vβ17/Null BiAbs. Vβ17+ cells frequency was normalized to target cells. Cells were cultured under 18% oxygen (Normoxia) or 2% Oxygen (Hypoxia). Target cell lysis was (% 7-AAD+ cells among CFSE labelled targets) assessed on day 3 and the abundance of Vβ17+ cells was assessed on day 5 of co-culture period.

### 7.1.18. Statistical Analysis

Statistical analysis was performed using GraphPad Prism version 8 (La Jolla, CA, USA). EC50 was calculated using a 4-parameter dose-response curve with the concentration on the x-axis (log scale) and specific lysis on the y-axis (linear scale). Paired t test was used to calculate the statistical significance for figures.

### 7.2. Example 2—Results and Analysis

### 7.2.1. Humanization and Epitope/Paratope Characterization of the Anti-TRBV19 Antibody

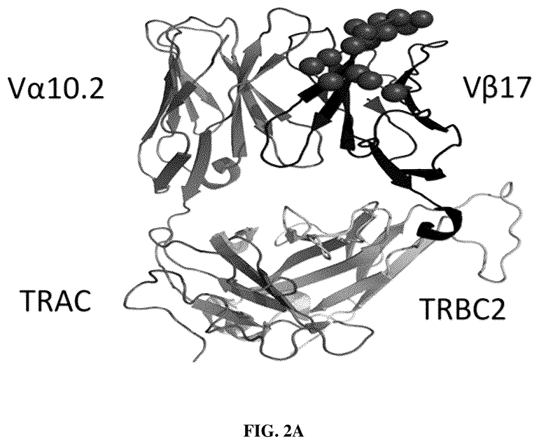

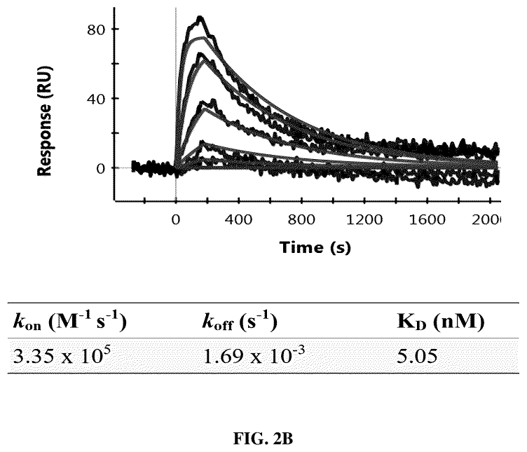

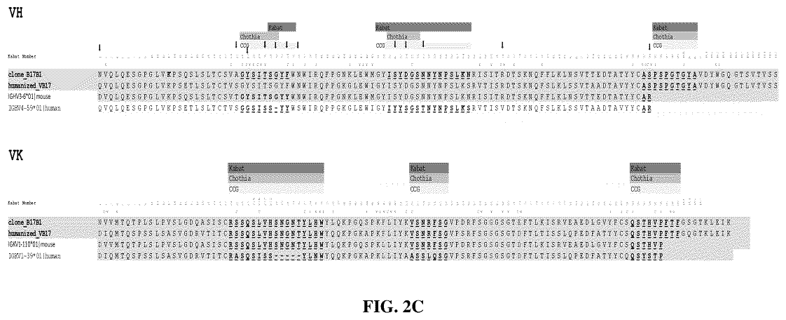

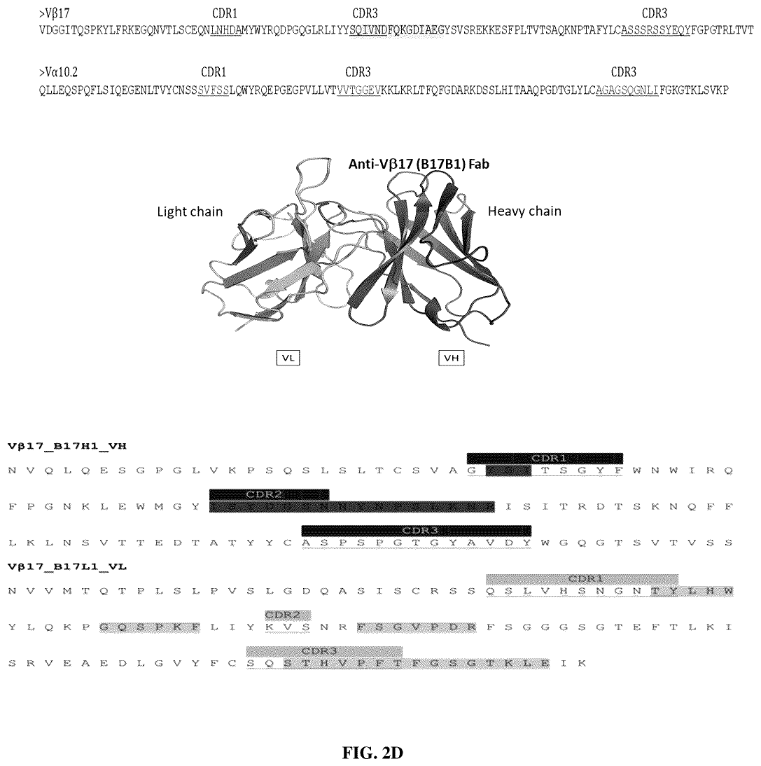

Mouse anti-human Vβ17 clone B17B1 binds to the antigen (Vβ17−Vα10.2 fused to human Fc) with a K_{D} or 5.05 nM (FIG. 2B). Humanization of murine B17B1 was performed following the approach outlined by Singh et al. Based on sequence conservation, the heavy chain germline IGHV4-59*01 was chosen for framework adaption (FIG. 2C). Three somatic hypermutation sites in the heavy chain were chosen for binary library design. For light chain frame adaption, IGKV1-110*01 was chosen as the best germline. The variants were cloned and expressed in E. coli. The supernatants were screened in single point ELISA and the periplasmic preparation was used for dose response. A mouse/human chimeric B17B1 Fab was used as parental control. Clone B17B21 maintained the binding activity similar to murine B17B1 and was converted to IgG for additional profiling.

To map the binding site on recombinant human Vα10.2-Vβ17TCR, epitope mapping was performed by HXMS technology. Clone B17B1 was incubated with an equimolar concentration of human Vα10.2-Vβ17TCR in a deuterated buffer. Human Vα10.2-Vβ17TCR protein alone served as the control. Extent of protection was inferred by measuring the differences in hydrogen/deuterium exchange between TCR alone or in complex with the B17B1. The protection map and further refinements of the analysis showed significant protection of regions identified as residues: ⁵⁶SIVNDFQKGDIAEG⁷⁵ on human Vα10.2-Vβ17TCR upon complexation (FIG. 2D). The epitope mapping indicated that the B17B1 antibody primarily bound to a portion of CDR2 and FR3 in the Vβ17 chain of the TCR. The paratope of this antibody/antigen complex was also determined (FIG. 2D).

### 7.2.2. Vβ17+ T Cells are Found in Healthy Humans and Cancer Patients

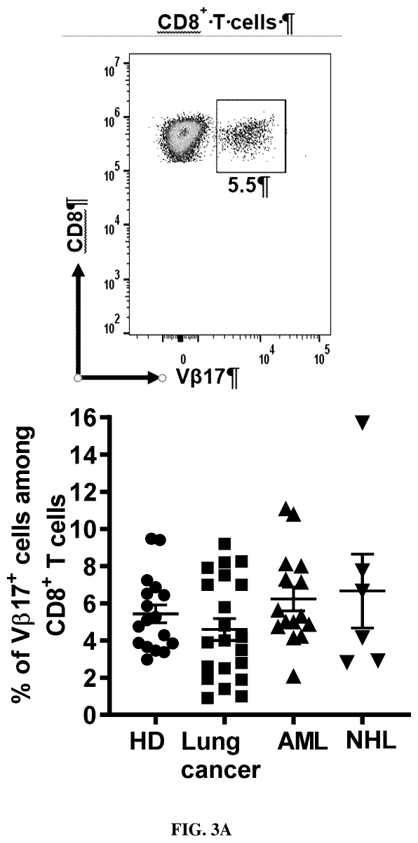

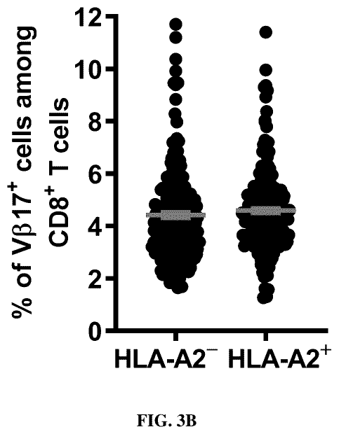

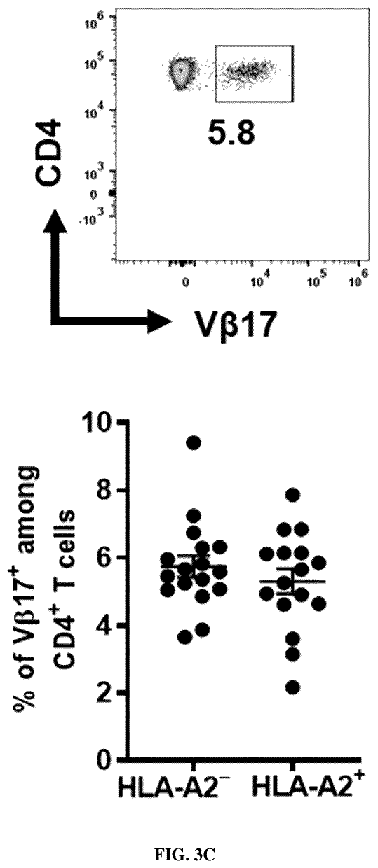

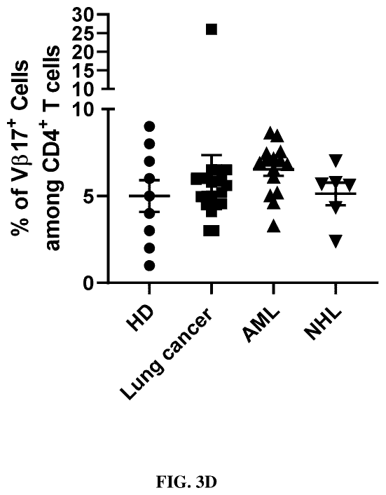

To examine whether Influenza A-specific T cells are present in normal human and cancer patients, this study focused on Vβ17+ T cell subset. Vβ17+CD8 T cells is a unique subset of T cells that are reactive to a dominant Influenza A Virus-derived M1-58-66 epitope and are part of a highly restricted CD8 memory T cell repertoire in HLA-A*0201+ humans^{8,15}. This study used anti-Vβ17 antibodies to stain PBMCs from healthy volunteers and cancer patients and determined the frequency of Vβ17+ T cells among the CD3 pan T cell population, among CD8+ T cells (FIGS. 3A-3B), among CD4+ T cells (FIGS. 3C-3D). The data suggests that all PBMC samples tested contained Vβ17+ T cells within T cell population, however, the frequency varied approx. 3 to 8% in total CD3 pool and between 1 to 12% among CD8+ T cell pool in healthy subjects (FIG. 3A). In addition, similar numbers of CD8+Vβ17+ T cells were seen in HLA-A2− and HLA-A2+ individuals (FIG. 3B). Vβ17+ T cells were also found in lung cancer and AML patient derived PBMC samples (FIG. 3A). Similar ranges of Vβ17+ T cells were seen in both CD4 and CD8 T cell compartment when compared to healthy subjects. These data suggest presence of Vβ17+ T cells among all human subjects including cancer patients.

7.2.3. Vβ17⁺CD8⁺ T Cells are Influenza Specific and can be Activated and Expanded Ex Vivo

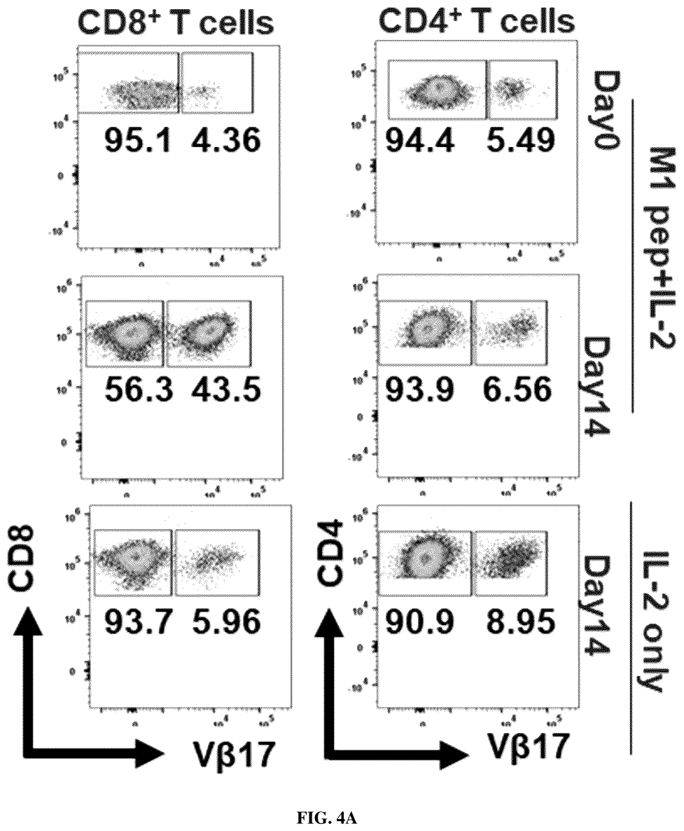

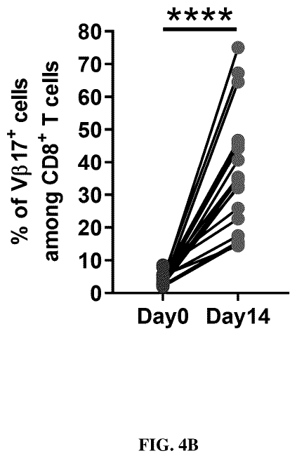

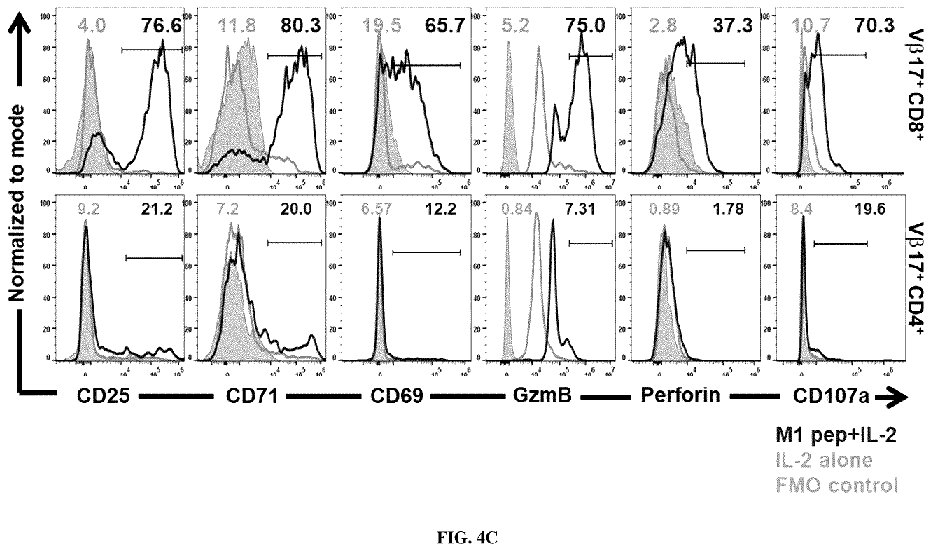

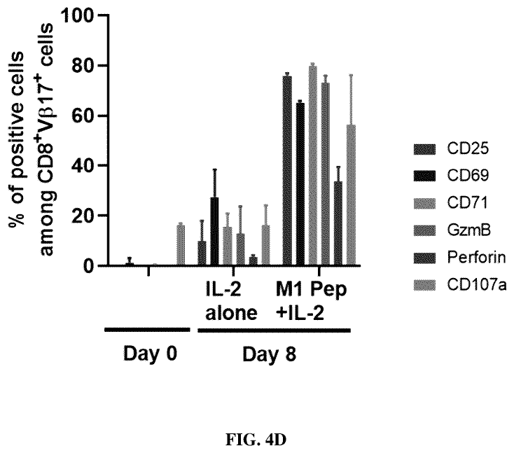

To examine whether Vβ17+ T cell are indeed Influenza A Virus specific, this study stimulated PBMCs obtained from HLA-A*0201+ healthy donors with Influenza A-derived M1-58-66 peptide and observed for expansion and upregulation of activation markers, and expression of effector molecules. Frequency of Vβ17+ cells among total CD8+ T cells was determined on day 14 of the culture period. The data suggests that frequency of Vβ17+ cells among total CD8+ T cells significantly increased in multiple donors upon culture with Influenza A-derived M1-58-66 peptide and no expansion was seen in CD4 compartment (FIGS. 4A-4B). This study further characterized Vβ17+CD8 T cells for their activation status using various markers of activation including CD25, CD71 and CD69. The data suggests that stimulation of CD8 T cells with Influenza A-derived M1-58-66 peptide results in selective activation of Vβ17+ cells as judged by expression of activation markers (FIGS. 4C-4D). In addition, this study found increased expression of effector molecules such as Granzyme B, Perforin and CD107a that are induced by activating Vβ17+ cells with Influenza A-derived M1-58-66 peptide (FIGS. 4C-4D). Taken together, these data demonstrate that Vβ17+ cells are indeed Influenza A specific and manifest CTL phenotype.

### 7.2.4. Vβ17 Bispecific Antibody Binds to Antigen Expressing Tumor Cells and Vβ17+CD8 T Cells

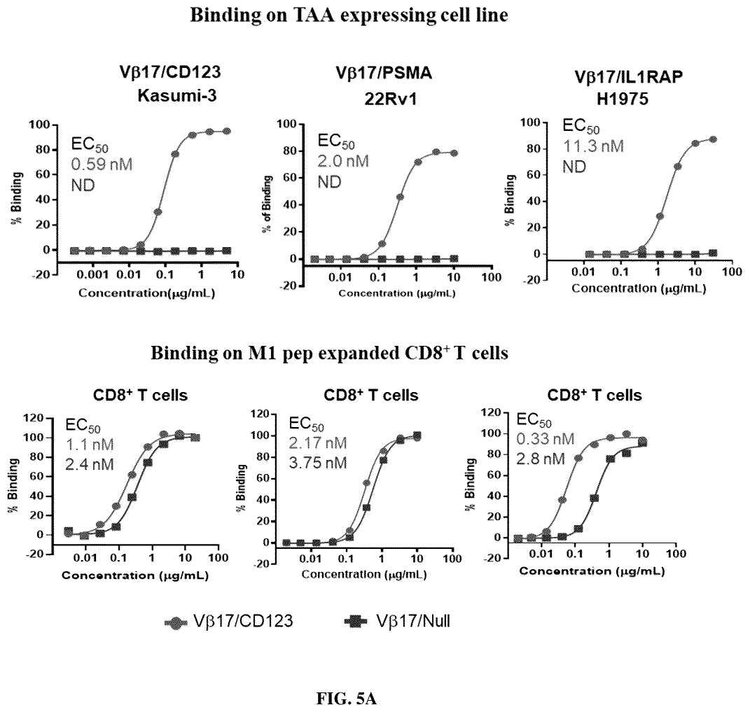

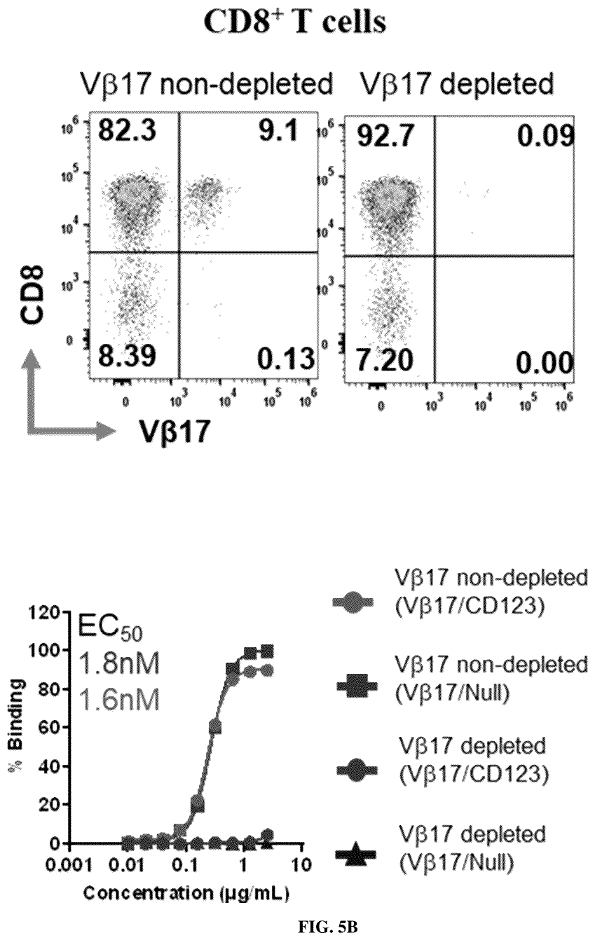

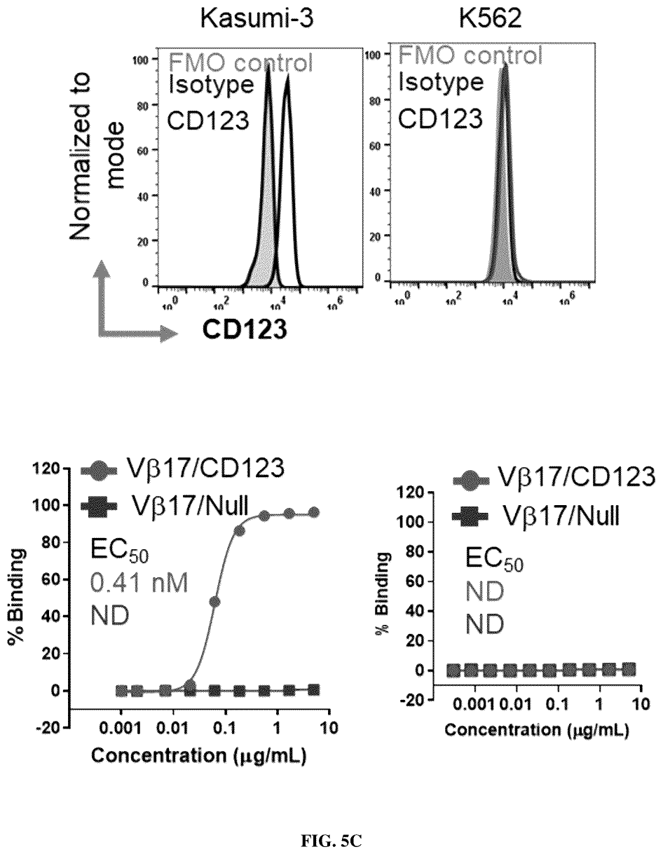

To understand the binding kinetics of Vβ17 bispecific antibodies, three bispecific molecules Vβ17/CD123, Vβ17/PSMA and Vβ17/IL1RAP were tested for binding kinetics to tumor cell surface expressed tumor antigens and to Vβ17+CD8 T cells. Vβ17/CD123 bispecific antibody and Kasumi-3 cells were incubated with Vβ17/CD123 and Vβ17/NULL arm control bispecific antibodies at various concentrations. Cell bound bispecific antibody was detected with mouse anti-human IgG4 Fc-PE secondary antibody and cells were analyzed by FACS method. FIG. 5A shows the binding kinetics of Kasumi-3 cells for Vβ17/CD123 bispecific antibody (top left panel) and the EC₅₀ for Vβ17/CD123 was determined to be 0.59 nM. Similarly, binding kinetics of Vβ17/PSMA bispecific antibody to 22Rv1 tumor cell line expressing PSMA was determined (top middle panel), and the EC₅₀ for Vβ17/PSMA was determined to be 2.0 nM. Binding kinetics of Vβ17/IL1RAP bispecific antibody to H1975 tumor cell line expressing ILIRAP was also determined (top right panel), and the EC₅₀ for Vβ17/IL1RAP was determined as 11.3 nM.

For determining the binding of bispecific molecules to Vβ17+CD8 T cells, enriched Influenza A Virus-derived M1-58-66 peptide stimulated CD8+T (from day 14 culture) cells were incubated with various concentrations of Vβ17-bispecific and Vβ17/NULL arm control antibodies. Mouse anti-human IgG4 Fc-PE secondary antibody was used to detect the bispecific antibody using FACS method. FIG. 5A shows the frequency of CD8+ T cells positive when treated with different concentration of bispecifics. The EC50 for Vβ17/CD123 was determined 2.4 nM (bottom left panel). Binding kinetics of Vβ17/PSMA bispecific antibody is shown in bottom middle panel and the EC50 for Vβ17/PSMA was determined to be 3.75 nM. Similarly, binding kinetics of Vβ17/IL1RAP bispecific antibody to Vβ17+CD8 T cells was determined (bottom right panel), and the EC50 for Vβ17XIL1RAP was determined to be 2.8 nM. The results presented here demonstrate tight binding of bispecific molecules to the tumor cell lines as well as to Vβ17+CD8 T cells and their suitability for serving as T cell engagers.

### 7.2.5. Vβ17 Bispecific Selectively Binds to Vβ17+ T Cells and Tumor Target Expressing Cell Line

Although above data established that Vβ17 bispecific molecules bind to Vβ17+CD8 T cells and to their respective tumor associated antigen, this study wanted to rule out if these bispecific molecules cross react to other non-Vβ17+ T cells or tumor cells. In this respect this study studied the binding of these molecules to CD8 T cells that were depleted of Vβ17+ T cells and tumor cells lacking tumor associated antigens. this study used Vβ17/CD123 bi-specific antibody to test this concept. Enriched Vβ17+CD8 T cells population or a population that were depleted of Vβ17+ T cells were used to determine selective binding to Vβ17+ T cells. To establish selective binding to tumor cells expressing CD123, this study used Kasumi-3 cell line that abundantly express CD123 and K562 cell line that does not express CD123. Data presented in FIG. 5B shows presence of Vβ17+ T cells among CD8+ T cells and lack of Vβ17+ T cells after depletion of Vβ17+ T cells (Upper left panels). Selective binding of Vβ17/CD123 to Vβ17+ T cells is shown in FIG. 5B. For selective tumor antigen binding expression of CD123 is shown in FIG. 5C where Kasumi cell lines is shown to express CD123 (upper left panel) and K562 cell line is lacking CD123 expression (upper right panel). Selective binding of Vβ17/CD123 to Kasumi cell line is shown in FIG. 5C (lower left panel) and lack of bind is shown to K562 cell line (lower right panel). These data clearly demonstrate that Vβ17/CD123 selectively bind to Vβ17+ T cells and CD123 expressing tumor cells.

### 7.2.6. Vβ17 Bispecific Mediated Redirection of T Cells Effectively Eliminates Liquid and Solid Tumors

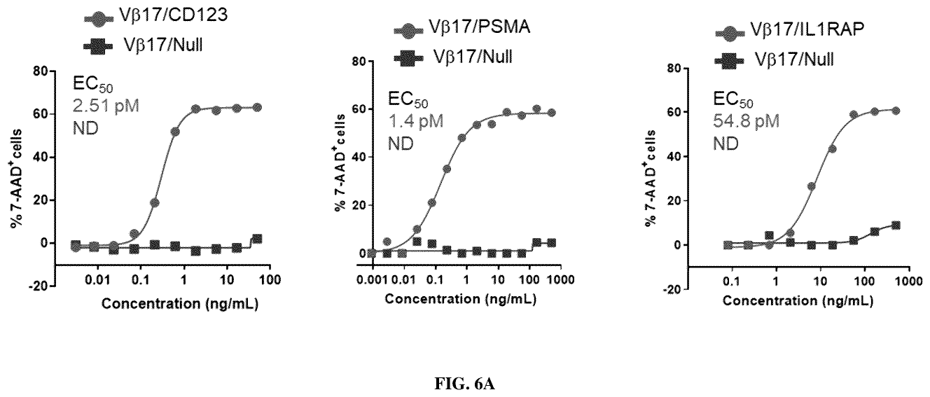

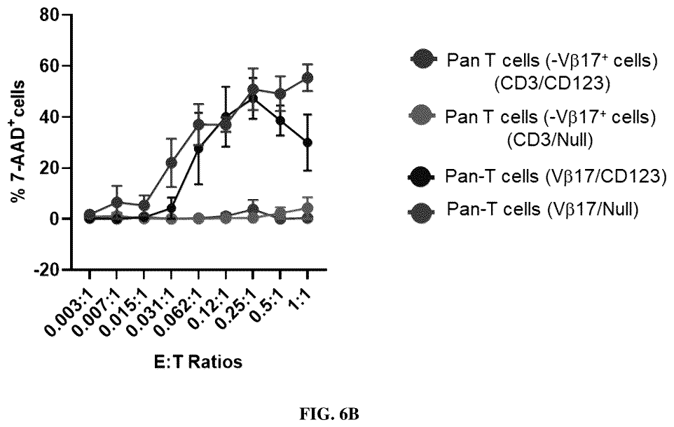

To determine if Vβ17-redirected T cells can be efficiently recruited and kill tumor cells this study enriched Vβ17⁺CD8⁺ T cells (effectors) by culturing pan CD8 T cells obtained from PBMCs from healthy donors with Influenza A Virus-derived M1-58-66 peptide. These enriched T cells were co-cultured with CFSE labelled target cells at 1:1 (Kasumi-3 cells as targets) and 5:1 (for 22Rv1 and H1975 cells as targets) ET ratio in the presence of indicated concentration of Vβ17 and Vβ17NULL arm control bispecific antibodies for a period of 24 hours (for Kasumi-3 targets) and 72 hours (for 22Rv1 and H1975 targets). Target cell lysis was determined by the 7-AAD staining and flow cytometry. FIG. 6A represents the frequency of specific target cell lysis at the indicated of concentration of Vβ17 bispecific antibodies and their respective Vβ17/NULL arm controls. EC50 values in pM rage were obtained in these experiments. The data suggests Vβ17 bispecific antibodies mediates efficient cytotoxicity against both solid and liquid tumor targets. This study further tested if selective redirection of Vβ17+ T cells is as potent as Pan T-cell redirection approach. Pan-T cells containing about 5% Vβ17+ T cells or Pan-T cells depleted of Vβ17+ were cultured with Kasumi-3 target cells in the presence of Vβ17/CD123 and CD3/CD123 bispecific antibodies respectively at indicated ET ratios. The data suggests that selective redirection of Vβ17+ T cells is as potent as Pan T-cell redirection in mediating cytotoxicity of tumor cells (FIG. 6B). These findings validate the concept that activation and redirection of a subset of viral specific T cells such as Vβ17+αβ cells using a bispecific antibody is has potential to eliminate tumors and their cytoxicity potential is similar to pan T cells.

### 7.2.7. Vβ17/CD123 Bispecific Antibody Potently Mediates Activation, Differentiation, Proliferation and Effector Functions of Vβ17+CD8+ T Cells Among Whole PBMCs

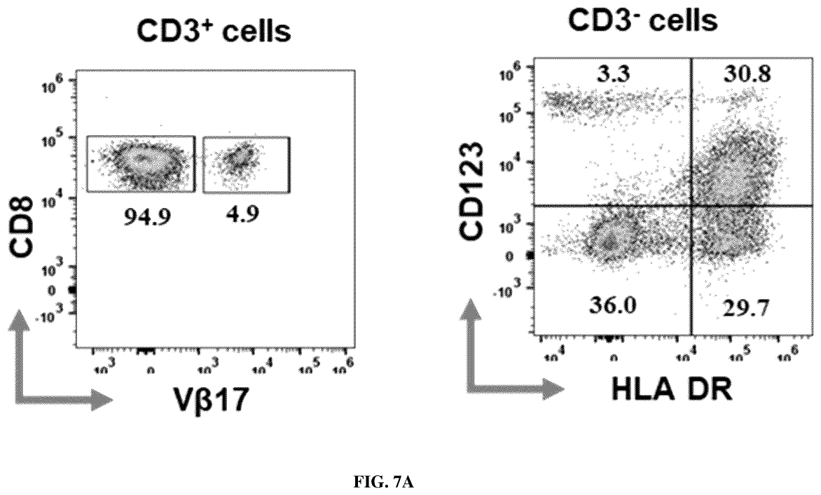

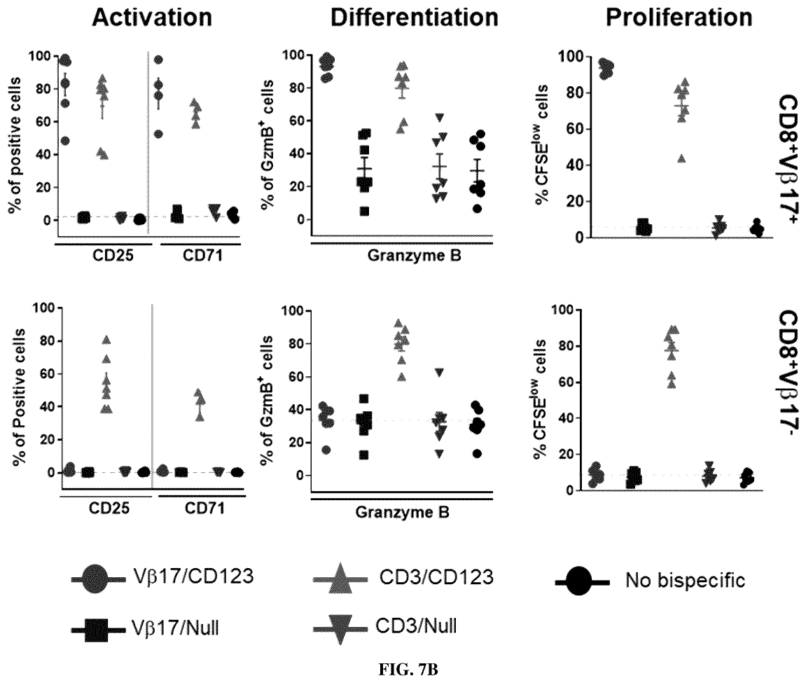

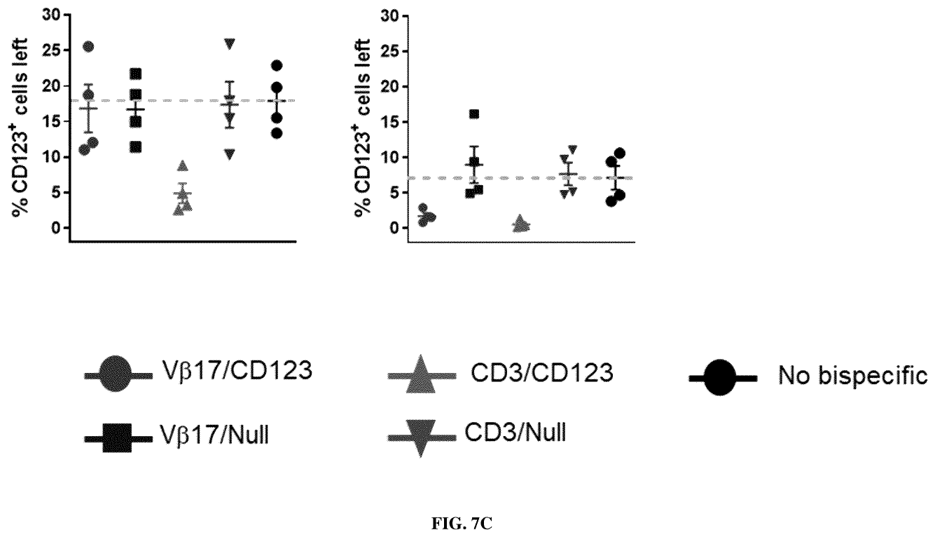

To further validate the concept that redirection of a small subset of Vβ17+ T cells are sufficient to mediate potent cytotoxicity against target cells this study leveraged the presence of target cells (CD123+ subset of monocyte) within bulk PBMC population to carry-out Vβ17 bispecific mediated cytotoxicity of target cells. This approach not only allows this study to work with a very natural system where no manipulation of target or effector cells is required, it also allows this study to measure overall cytokine release by various populations of cells present in PBMCs. Whole PBMCs were cultured in the presence bispecific antibodies and as a control whole PBMCs were cultured in the absence of any bispecific antibody to determine the elimination of CD123+ target cells. Data presented in FIGS. 7A-7C indicated Vβ17/CD123 bispecific antibody potently mediates activation, differentiation, proliferation and effector functions of Vβ17+ T cell subset among whole PBMCs, whereas, CD3/CD123 bispecific antibody potently mediates activation, differentiation, proliferation and effector functions of all T cells including both Vβ17+ and Vβ17-negative T cells. Data further indicates almost complete elimination of CD123+ targets cells by Vβ17/CD123 bispecific antibody, however, kinetics of killing was slower. For example, there was not much killing of target cells after day one of the culture with Vβ17/CD123 (FIG. 7C), whereas, CD3/CD123 mediated complete removal of the target cells. However, on day 5 of the culture elimination of the CD123+ target cells were equivalent in both cases. Overall, the data suggests recruitment of a small subset (˜5%) of Vβ17+ T cells are sufficient to mediate efficient cytotoxicity of target cells and further reinforces the potency of this subset of T cells.

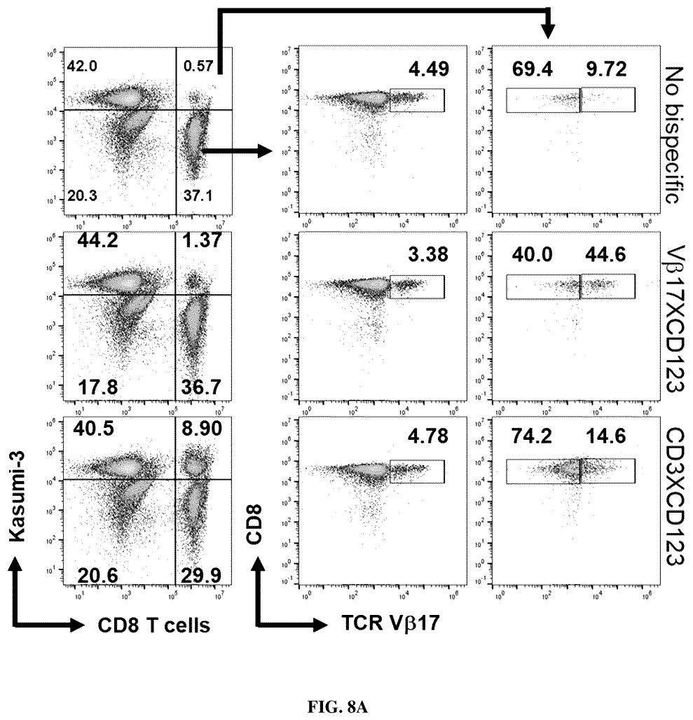

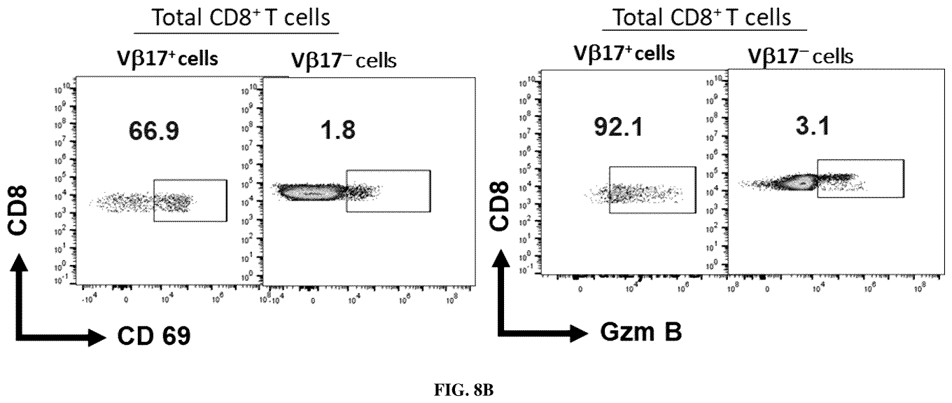

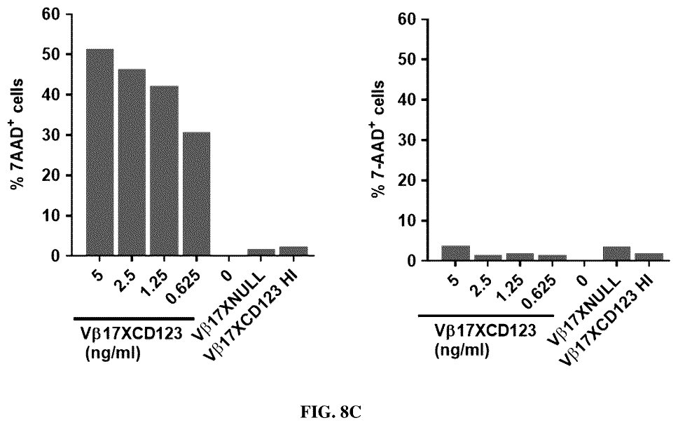

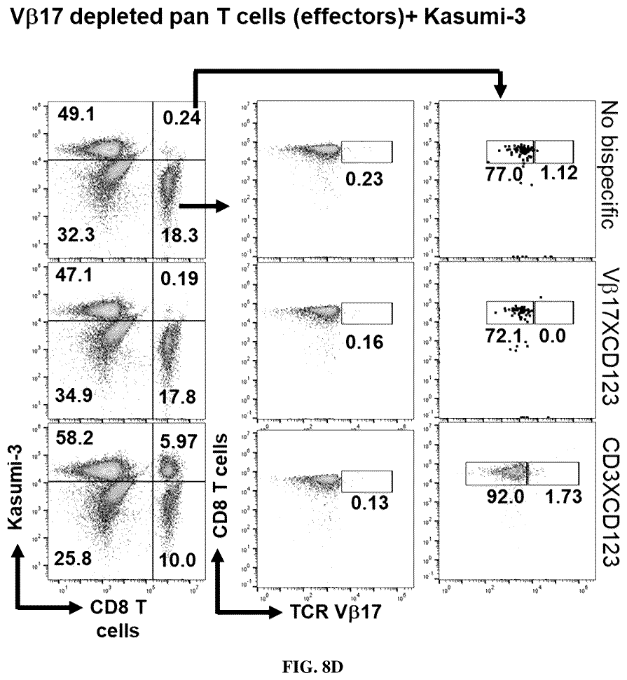

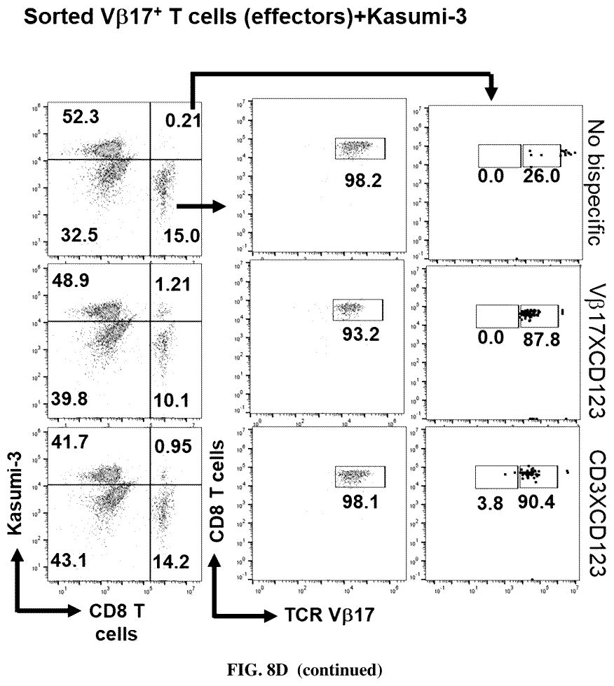

As shown in FIGS. 8A-8D, Vβ17/CD123 bispecific antibody selective recruits, activates Vβ17⁺ T cells and mediates their cytotoxicity.

### 7.2.8. Vβ17+ T Cell Selective Redirection do not Overt Cytokine Release Compared to Pan T Cell Redirection

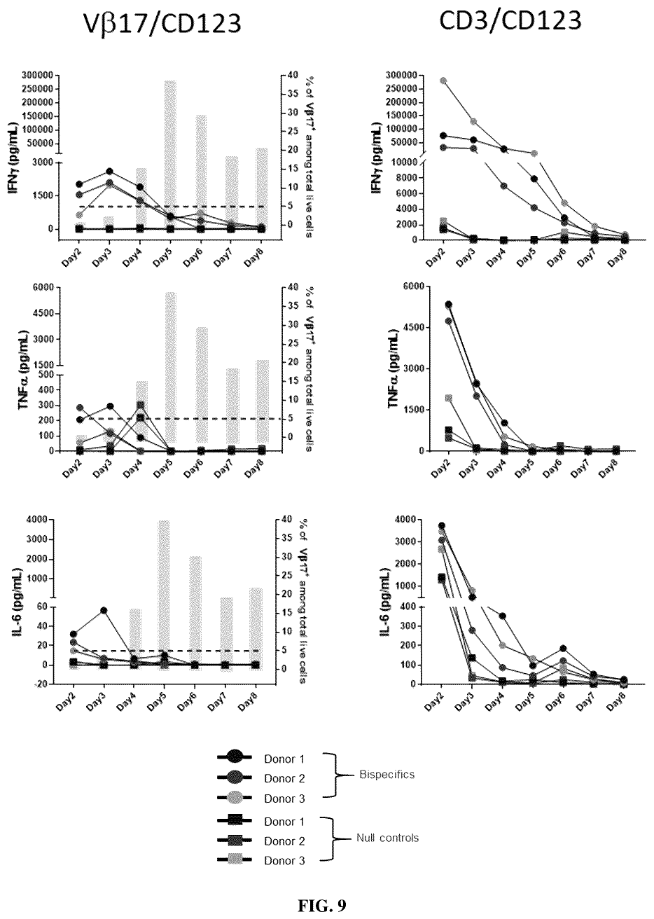

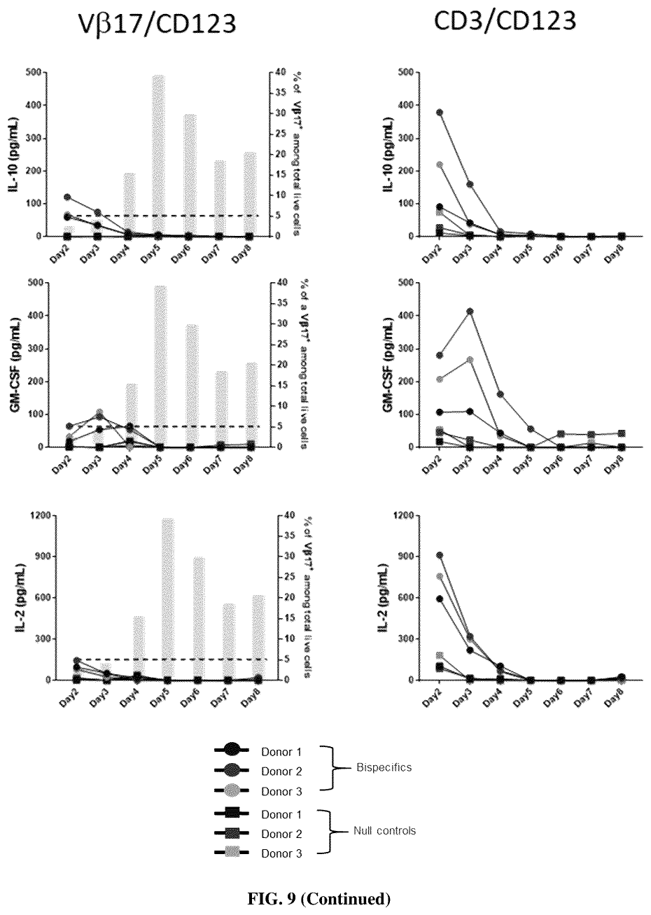

In order to determine cytokine production by whole PBMCs when treated with Vβ17/CD123 and CD3/CD123, this study cultured PBMC in the presence of bispecific antibodies at a concentration of 5 ng/mL or in the absence of bispecific antibody to serve as a control. Cytokine release was assessed from day 3 to day 8 cell culture supernatant. Data presented in FIG. 9 represent concentration of various cytokines in culture supernatant of whole PBMCs stimulated with indicated bispecific antibodies for three donors. As expected, there was an overt cytokine release with CD3/CD123 bispecific antibody (right panels) suggesting pan activation of T cells, where is little to no cytokine release with Vβ17/CD123 bispecific antibody (left panels). It is interesting to note that there was no cytokine release even when there was a high proliferation of Vβ17+ T cells (grey bars in left panels).

### 7.2.9. Vβ17/CD123 Bispecific Antibody Effectively Controlled KG1 Tumor Cell Growth in a Xenograft Model.

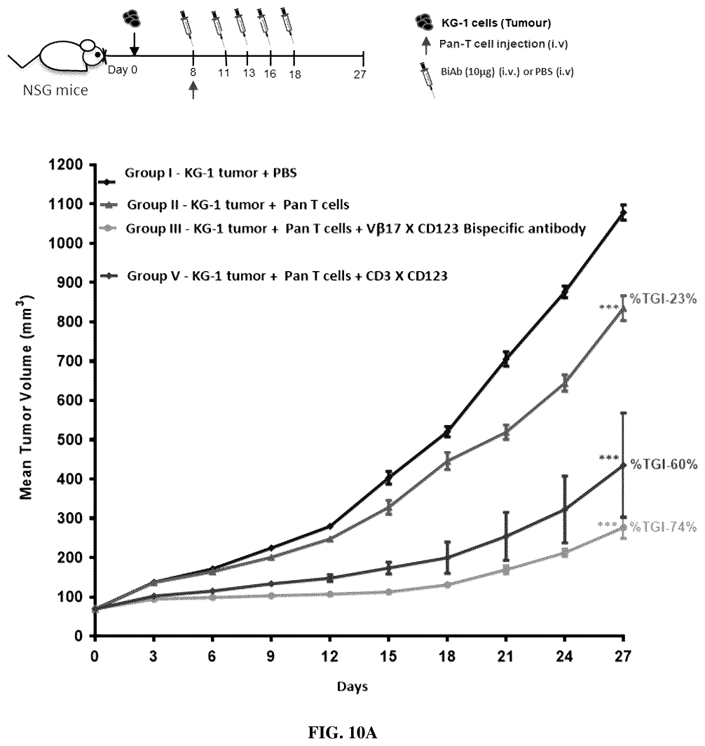

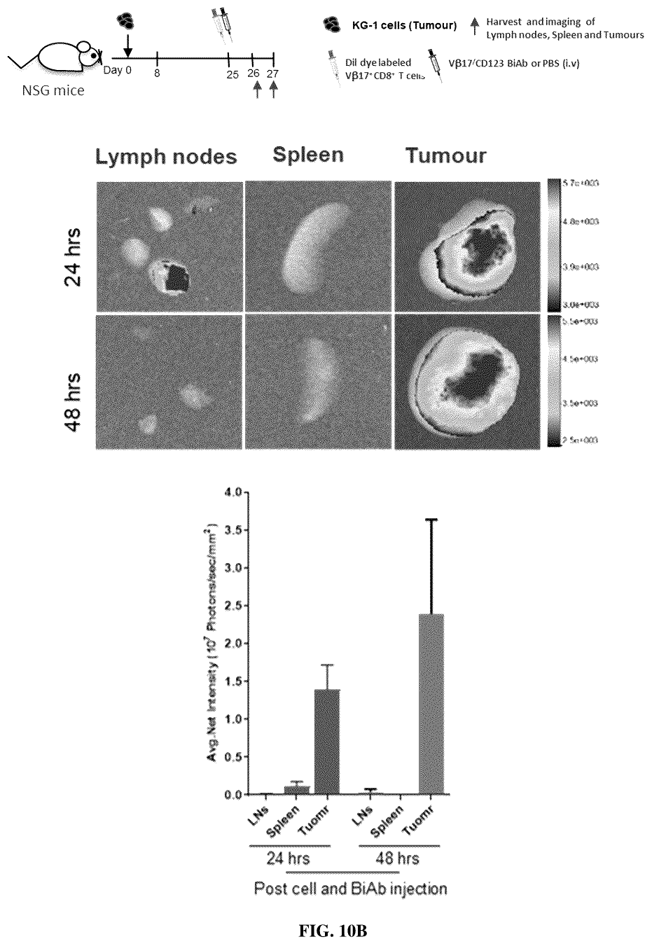

NSG mice were subcutaneously injected with KG-1 cells as described in detail in methods section. Seven days post tumor implant, mice were randomized based on the tumor size (˜50-70 mm³) and segregated into groups. After the randomization (Day 1), twenty million enriched Pan-T cells were intravenously administered to each mouse, followed by an i.v. injection of 10 μg of bispecific antibody every other day for 5 does. Tumor volume (mm3) was measured at indicated time points as mentioned in the methods section. The data presented in FIG. 10A indicates that selective recruitment of Vβ17+ T cells by Vβ17/CD123 bispecific antibody showed similar efficacy to CD3/CD123 bispecific antibody. Moreover, established KG-1 tumour (around 500-600 mm3; 25 days post KG-1 cells injection) bearing NSG mice were intravenously injected with DIL dye labelled enriched CD8+Vβ17+ T cells (around 6 million cells/mouse), followed by an i.v. injection of 10 μg of Vβ17/CD123 bispecific antibody. Post 24 and 48 hours injection of bispecific antibody, mice were euthanized and organs were ex-vivo imaged. Fluorescence intensity in representative images of FIG. 10B refers to the abundance of adoptively transferred Dil dye-labelled Vβ17⁺CD8⁺ T cells in various organs of mice.

These data further suggest that selective redirection of only ˜5% T cells (Vβ17+) is as potent as Pan T-cell redirection approach in mediating tumor cell cytotoxicity in vivo. These findings validate the concept that activation and redirection of a subset of viral specific T cells such as Vβ17+αβ cells using a bispecific antibody is has potential to eliminate tumors in vivo.

### 7.2.10. Vβ17/CD123 Bispecific Antibody Selectively Activates and Mediates Metabolic Modulation of Vβ17+ Cells

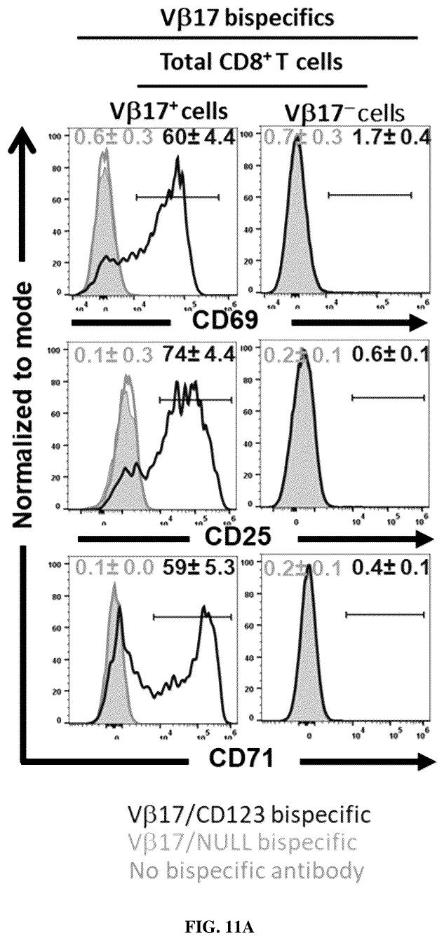

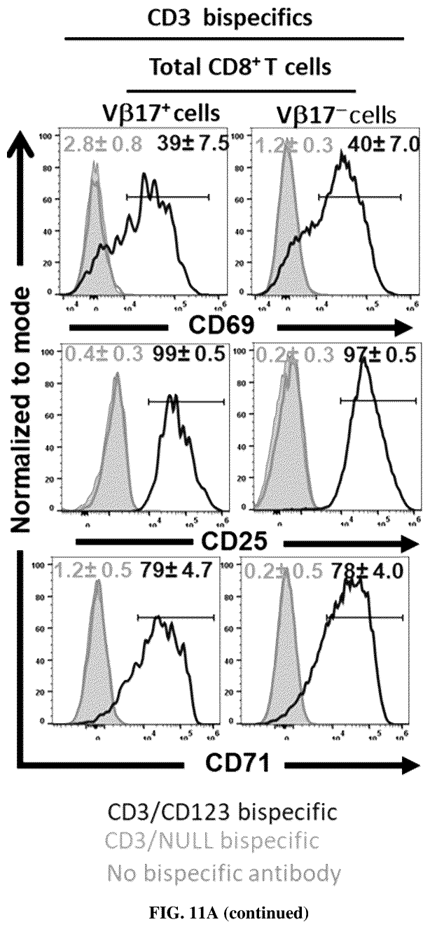

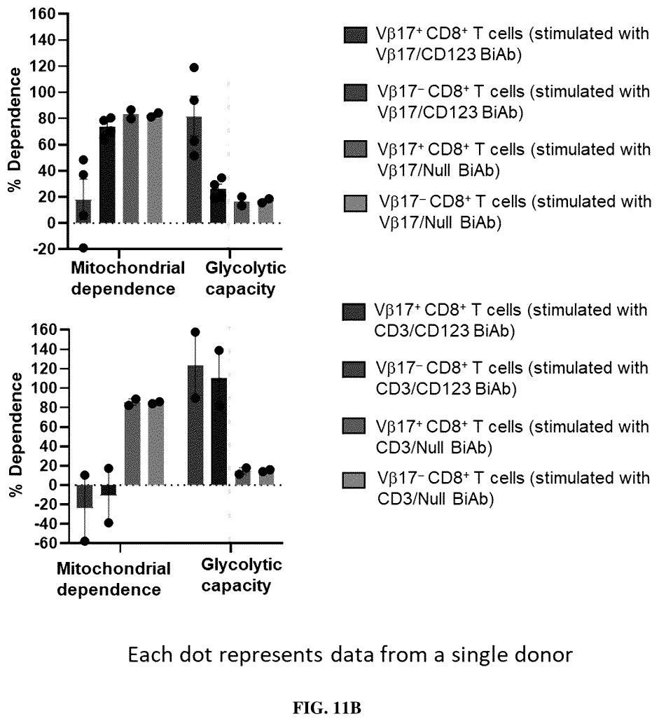

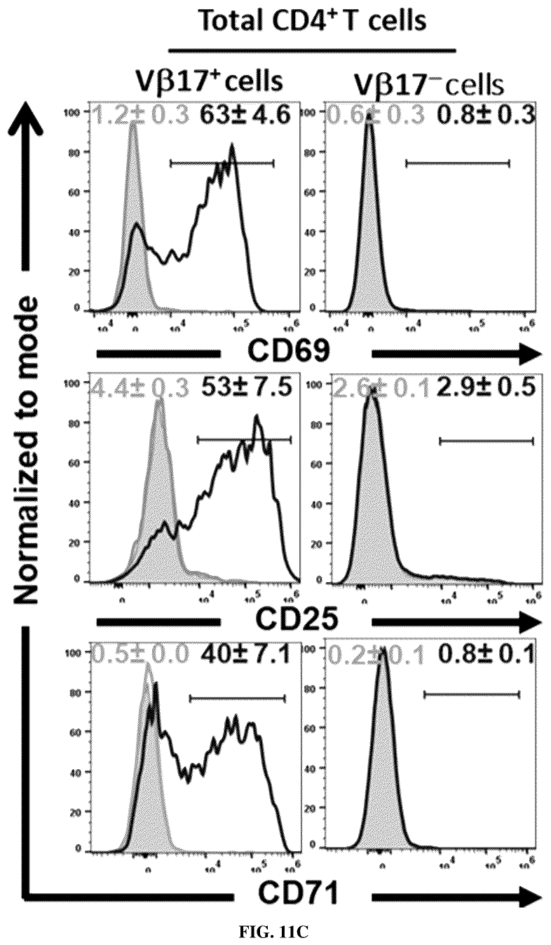

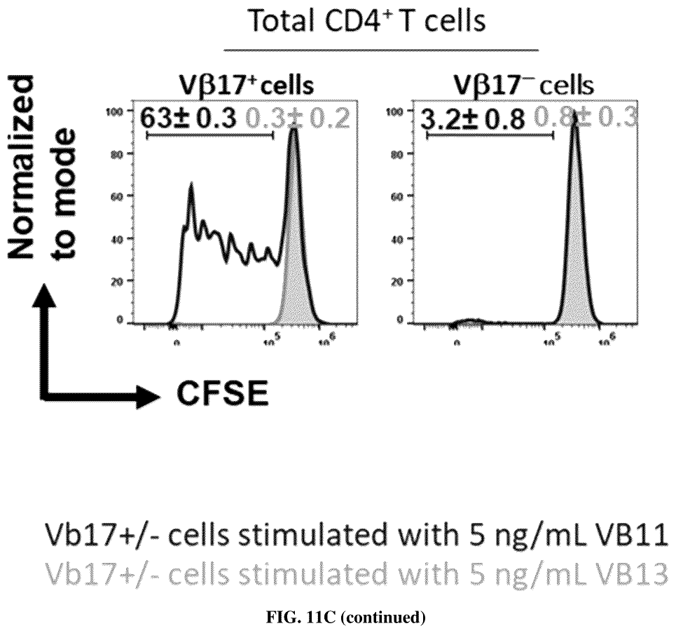

Pan-T cells (effectors) were co-cultured with Kasumi-3 (targets) cells in the presence or absence of indicated bispecific antibody for 72 hrs. Gates in histogram overlays of FIG. 11A refer to the mean frequency (±SEM) of Vβ17⁺ and Vβ17⁻CD8 T cells that were positive for CD25, CD69 and CD71 (activation) surface expression in the absence or presence of Vβ17/CD123, Vβ17/Null (left) and CD3/CD123, CD3/Null (right) BiAbs. Enriched CD8⁺ T cells (Effectors) were co-cultured with Kasumi-3 (targets) cells for 72 hours in the presence of Vβ17/CD123, CD3/CD123 and Null arm control BiAbs. Metabolic profiling of CD8⁺ T cells was carried out as described in materials and methods. Bars in FIG. 11B refer to the mean frequency (±SEM) of Vβ17⁺ and Vβ17⁻ CD8 T cells dependence on Mitochondria or Glycolysis in the presence of indicated BiAbs or their respective Null arm controls. n=4 healthy donors for BiAbs and 2 donors for Null arm controls from 2 independent experiments. Similarly, Gates in histogram overlays of FIG. 11C refer to the mean frequency (±SEM) of Vβ17⁺ and Vβ17⁻ CD4 T cells that were positive for CD25, CD69 and CD71 (activation) surface expression and CFSE dilution (proliferation) in the presence or absence of BiAbs.

### 7.2.11. Low Donor Heterogeneity in Vβ17/CD123 Bispecific Mediated Pan-T Cell Cytotoxicity

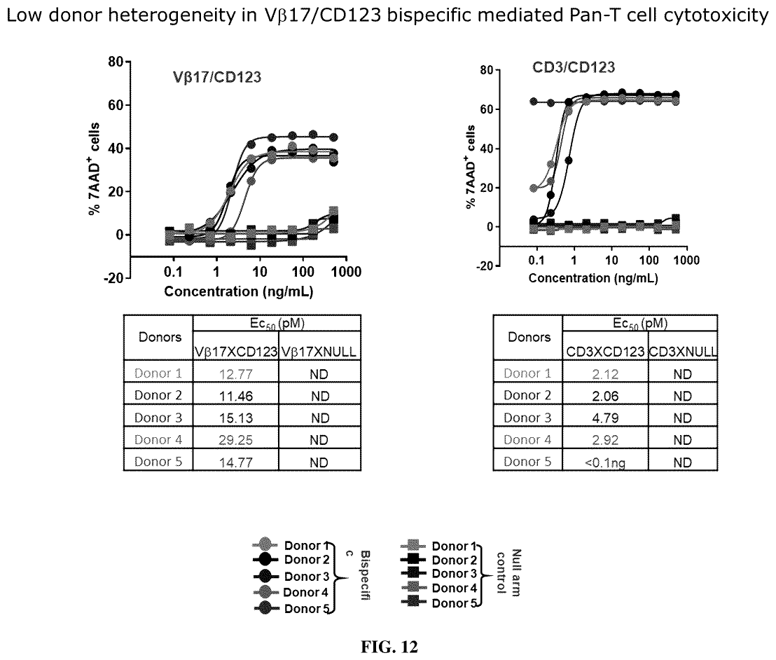

Pan-T cells were enriched from healthy donors and were cultured with targets (Kasumi-3) cells in the presence of Vβ17/CD123 or CD3/CD123 along with their respective Null arm controls for 72 hours. Graphs within FIG. 12 refer to the percentage of specific target cell lysis (% 7-AAD+ cells) in the presence of Vβ17/CD123 (left) or CD3/CD123 (right) biAbs and their respective null arm controls. n=5 donors from a single experiment. Table within FIG. 12 shows EC50 values for BiAbs and their respective Null arm controls. ND: Not Determined.

7.2.12. Vβ17⁺ T Cell Redirection Show Durable Cytotoxicity with Minimal Cellular Senescence/Exhaustion

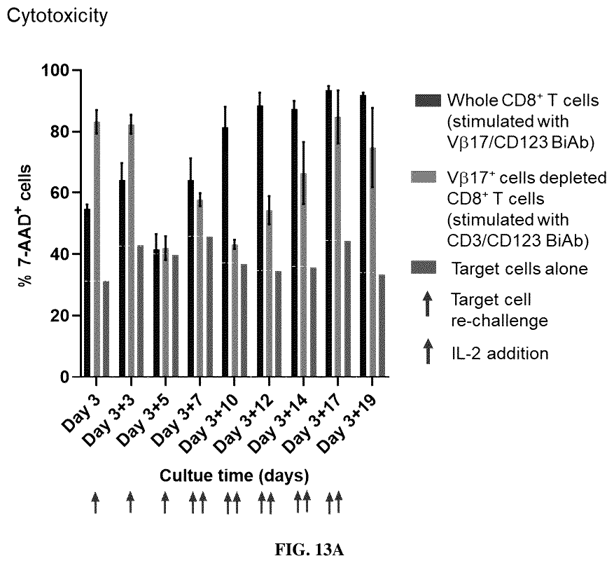

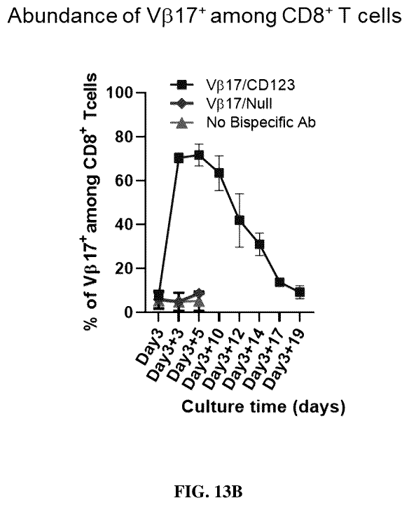

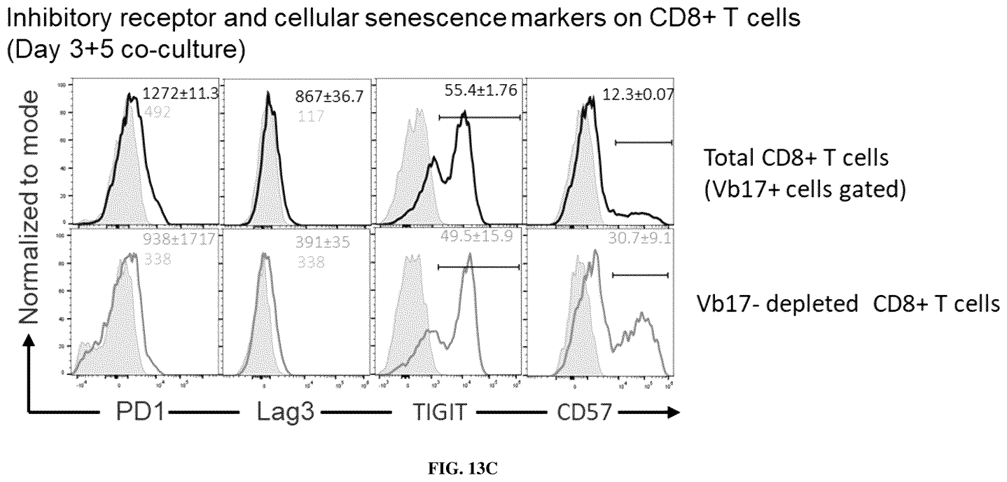

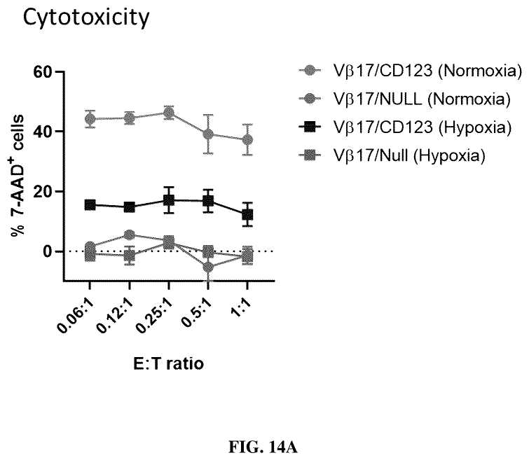

Enriched CD8 T cells, CD8 T cells (Vβ17depleted) were co-cultured with Kasumi-3 cells (targets) in the presence of Vβ17/CD123 and CD3/CD123 BiAbs respectively for indicated time points. Detailed experimental method is outlined in methods section. FIG. 13A refers to the frequency (mean±SEM) of target cell killing (% 7-AAD+ among CFSE/CTV labelled target cells) on indicated time points (days) in the presence of Vβ17/CD123 and CD3/CD123 BiAb respectively. Dotted line refers to target alone cell death (green bars). Arrows (red) mirror the target (Kasumi-3) cell re-challenge on indicated time points. Arrows (violet) point to IL-2 addition to the culture wells on indicated time points. n=4 donors from 2-3 independent experiments. FIG. 13B depicts the frequency (mean±SEM) of Vβ17+ cells among CD8 T cells on indicated days of co-culture period from a (CD8 T cells co-culture with target cells in the presence of Vβ17/CD123 BiAb). (c). Values in histogram overlays of FIG. 13C refer to the mean (±SEM) gMFI (for PD1, Lag3) or frequency (for TIGIT and CD57) of Vβ17+CD8⁺ T cells or Vβ17−CD8+ cells.

### 7.2.13. Vβ17+ T Cells Retain Effector and Proliferative Abilities, Albeit Less, Under Hypoxia Conditions

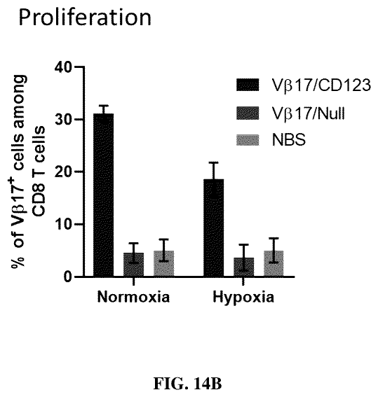

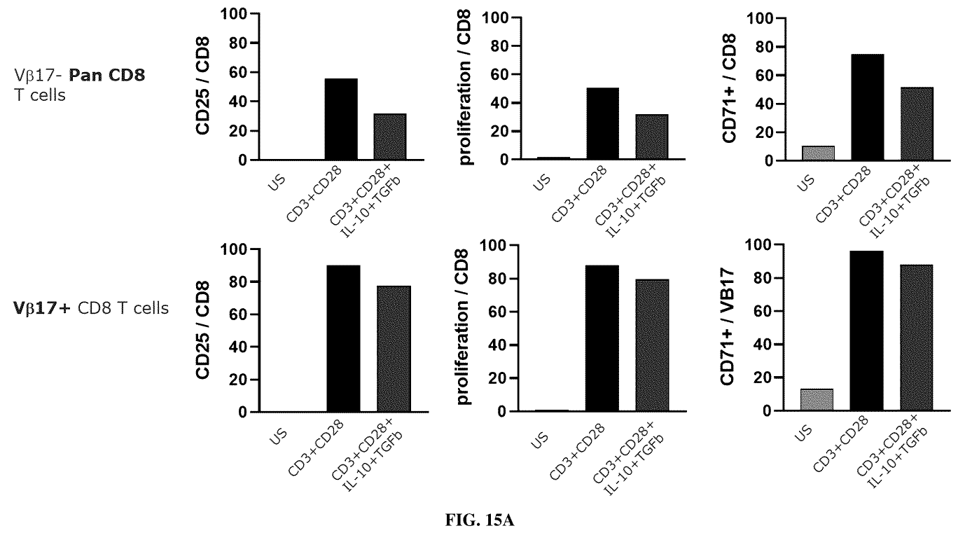

Enriched CD8⁺ T cells, CD8⁺ T cells (depleted of Vβ17⁺ cells) were co-cultured with Kasumi-3 (Target) cells in the presence of Vβ17/CD123 and CD3/CD123 BiAbs respectively at normoxia (18%) and Hypoxia (2%) conditions for 72 hrs (cytotoxicity) 150 hrs (Proliferation). FIG. 14A refers to the frequency (mean±SEM) of specific target cell lysis (cytotoxicity) under normoxia/hypoxia at indicated ET ratios with the indicated BiAbs. n=5 donors from two independent experiments. Bars in FIG. 14B show the frequency of Vβ17⁺ cells among CD8 T cells on day 5 of co-culture under normoxia or hypoxia with indicated BiAb/Control at an ET ratio of 1:1. n=3 donors for a single experiment.

### 7.2.14. Activation and Proliferation of CD8+ are not Regulated by Suppressive Cytokines or M2 Macrophages

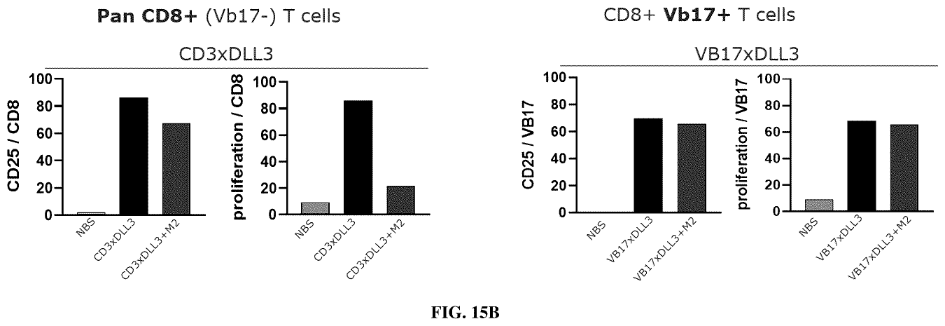

As shown in FIGS. 15A-15B, the activation and proliferation of CD8+ (Vβ17+ and Vβ17− T cells) are not regulated by suppressive cytokines like IL-10 and TGFβ or M2 macrophages.

From the foregoing, it will be appreciated that, although specific embodiments have been described herein for the purpose of illustration, various modifications may be made without deviating from the spirit and scope of what is provided herein. All of the references referred to above are incorporated herein by reference in their entireties.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

1. A pharmaceutical composition comprising: (i) an isolated population of cells comprising Vβ17+CD8+ T cells, (ii) one or more T cell engagers; and (iii) a pharmaceutically acceptable excipient.

2. The pharmaceutical composition of claim 1, wherein the isolated population of cells comprising Vβ17+CD8+ T cells has been activated or enriched via contacting a M1 peptide derived from human influenza A virus (M1₅₈₋₆₆) with a population of cells comprising T cells, optionally wherein the M1 peptide comprises the amino acid sequence of GILGFVFTL (SEQ ID NO:1).

3. (canceled)

4. The pharmaceutical composition of claim 1, wherein the isolated population of cells comprising Vβ17+CD8+ T cells has been activated or enriched via further contacting IL-2 with the population of cells comprising T cells.

5. The pharmaceutical composition of claim 1, wherein the population of cells comprising T cells has been cultured ex vivo in a medium comprising the M1 peptide and/or IL-2, optionally wherein the ex vivo culturing comprises:
(i) culturing the population of cells comprising T cells ex vivo in a medium comprising the M1 peptide and IL-2;
culturing the population of cells comprising T cells ex vivo in a medium comprising the (ii) M1 peptide, and then culturing the population of cells comprising T cells ex vivo in a medium comprising IL-2; or
(iii) culturing the population of cells comprising T cells ex vivo in a medium comprising the M1 peptide, then culturing the population of cells comprising T cells ex vivo in a medium comprising the M1 peptide and IL-2; and then culturing the population of cells comprising T cells ex vivo in a medium comprising IL-2.

6. (canceled)

7. The pharmaceutical composition of claim 5, wherein the population of cells comprising T cells has been cultured ex vivo for at least 1, 2, 3, 4, 5, 6, 7, 8, 9, 10, 11, 12, 13, or 14 days.

8. The pharmaceutical composition of any one of claim 2, wherein the population of cells comprising T cells is a population of whole peripheral blood mononuclear cells (PBMCs), optionally wherein the population of the whole PBMCs is from a healthy donor or an unhealthy donor.

9. (canceled)

10. The pharmaceutical composition of claim 1, wherein the Vβ17+CD8+ T cells have been isolated from the population of cells comprising T cells after contacting the population of cells comprising T cells with the M1 peptide and/or IL-2.

11. The pharmaceutical composition of claim 1, wherein the percent of Vβ17+CD8+ T cells in the isolated population of cells is more than 6%, 10%, 15%, 20%, 25%, 30%, 35%, 40%, 45%, 50%, 55%, 60%, 65%, 70%, 75%, 80%, 85%, or 90%.

12. The pharmaceutical composition of claim 1, wherein at least part of the Vβ17⁺CD8⁺ T cells express a cell surface receptor capable of binding to the M1 peptide.

13. The pharmaceutical composition of claim 1, wherein each of the T cell engagers is a multi-specific antibody, optionally wherein the multi-specific antibody comprises:
1) a first binding domain that binds to an antigen expressed on a Vβ17+CD8+ T cell, wherein the antigen expressed on the Vβ17+CD8+ T cell is T Cell Receptor Beta Variable 19 (TRBV19) or Vβ17; and
2) a second binding domain that binds to an antigen expressed on an unhealthy cell, wherein the antigen expressed on the unhealthy cell is a tumor-associated antigen (TAA), further optionally wherein the TAA is CD123, IL1RAP, PSMA, or B7H3.

14.-15. (canceled)

16. The pharmaceutical composition of claim 13, wherein the unhealthy cell is a cancer cell, a blood cancer cell, or a solid tumor cancer cell.

17.-19. (canceled)

20. The pharmaceutical composition of claim 1, wherein each of the T cell engagers is a TRBV19xTAA or Vβ17xTAA bi-specific antibody.

21. A method for treating a disease or disorder in a subject in need thereof, comprising administering to the subject the pharmaceutical composition of claim 1.

22. The method of claim 21, wherein the disease or disorder is cancer, optionally wherein the cancer is a blood cancer or a solid tumor cancer.

23. (canceled)

24. A method for:
(i) redirecting a T cell to a target cell, comprising contacting the target cell with one or more T cell engagers in the presence of an isolated population of cells comprising Vβ17+CD8+ T cells, wherein the contacting directs the Vβ17+CD8+ T cell to the target cell;
(ii) inhibiting the growth or proliferation of a target cell, comprising contacting the target cell with one or more T cell engagers in the presence of an isolated population of cells comprising Vβ17+CD8+ T cells, wherein the contacting results in the inhibition of the growth or proliferation of the target cell; or
(iii) eliminating a target cell, comprising contacting the target cell with one or more T cell engagers in the presence of an isolated population of cells comprising Vβ17+CD8+ T cells, wherein the contacting results in the elimination of the target cell.

25.-26. (canceled)

27. The method of claim 24, wherein the isolated population of cells comprising Vβ17+CD8+ T cells has been activated or enriched via contacting a M1 peptide derived from human influenza A virus (M1₅₈₋₆₆) with a population of cells comprising T cells, optionally wherein the M1 peptide comprises the amino acid sequence of GILGFVFTL (SEQ ID NO:1).

28. (canceled)

29. The method of claim 24, wherein the isolated population of cells comprising Vβ17+CD8+ T cells has been activated or enriched via further contacting IL-2 with the population of cells comprising T cells.

30. The method of claim 24, wherein the population of cells comprising T cells has been cultured ex vivo in a medium comprising the M1 peptide and/or IL-2, optionally wherein the ex vivo culturing comprises:
(i) culturing the population of cells comprising T cells ex vivo in a medium comprising the M1 peptide and IL-2;
(ii) culturing the population of cells comprising T cells ex vivo in a medium comprising the M1 peptide, and then culturing the population of cells comprising T cells ex vivo in a medium comprising IL-2; or
(iii) culturing the population of cells comprising T cells ex vivo in a medium comprising the M1 peptide, then culturing the population of cells comprising T cells ex vivo in a medium comprising the M1 peptide and IL-2; and then culturing the population of cells comprising T cells ex vivo in a medium comprising IL-2;
wherein the population of cells comprising T cells has been cultured ex vivo for at least 1, 2, 3, 4, 5, 6, 7, 8, 9, 10, 11, 12, 13, or 14 days.

31.-32. (canceled)

33. The method of claim 27, wherein the population of cells comprising T cells is a population of whole peripheral blood mononuclear cells (PBMCs), wherein the population of the whole PBMCs is from a healthy donor or an unhealthy donor.

34.-36. (canceled)

37. The method of claim 24, wherein at least part of the Vβ17+CD8+ T cells express a cell surface receptor capable of binding to the M1 peptide.

38. The method of claim 24, wherein each of the T cell engagers is a multi-specific antibody, optionally wherein the multi-specific antibody comprises:
1) a first binding domain that binds to an antigen expressed on a Vβ17+CD8+ T cell, wherein the antigen expressed on the Vβ17+CD8+ T cell is T Cell Receptor Beta Variable 19 (TRBV19) or Vβ17; and
2) a second binding domain that binds to an antigen expressed on an unhealthy cell, wherein the antigen expressed on the unhealthy cell is a tumor-associated antigen (TAA), further optionally wherein the TAA is CD123, IL1RAP, PSMA, or B7H3;
wherein the unhealthy cell is a cancer cell, a blood cancer cell, or a solid tumor cancer cell.

39.-45. (canceled)


SEQUENCE LISTING (83 sequences)

SEQ ID NO: 1 - protein, 9 aa, synthetic construct
  REGION 1..9: note = M1 peptide
GILGFVFTL

SEQ ID NO: 2 - protein, 12 aa, synthetic construct
  REGION 1..12: note = M1 peptide
KGILGFVFTLTV

SEQ ID NO: 3 - protein, 11 aa, synthetic construct
  REGION 1..11: note = M1 peptide
KGILGFVFTLT

SEQ ID NO: 4 - protein, 10 aa, synthetic construct
  REGION 1..10: note = M1 peptide
KGILGFVFTL

SEQ ID NO: 5 - protein, 11 aa, synthetic construct
  REGION 1..11: note = M1 peptide
GILGFVFTLTV

SEQ ID NO: 6 - protein, 10 aa, synthetic construct
  REGION 1..10: note = M1 peptide
GILGFVFTLT

SEQ ID NO: 7 - protein, 11 aa, synthetic construct
  REGION 1..11: note = VH CDR1
GYSITSGYFWN

SEQ ID NO: 8 - protein, 9 aa, synthetic construct
  REGION 1..9: note = VH CDR2
YISYDGSNN

SEQ ID NO: 9 - protein, 11 aa, synthetic construct
  REGION 1..11: note = VH CDR3
PSPGTGYAVDY

SEQ ID NO: 10 - protein, 16 aa, synthetic construct
  REGION 1..16: note = VL CDR1
RSSQSLVHSNGNTYLH

SEQ ID NO: 11 - protein, 7 aa, synthetic construct
  REGION 1..7: note = VL CDR2
KVSNRFS

SEQ ID NO: 12 - protein, 9 aa, synthetic construct
  REGION 1..9: note = VL CDR3
SQSTHVPFT

SEQ ID NO: 13 - protein, 700 aa, synthetic construct
  REGION 1..700: note = B17B21
DIQMTQSPSSLSASVGDRVTITCRSSQSLVHSNGNTYLHWYQQKPGKAPKFLIYKVSNRFSGVPSRFSGSGSGTDFTLTISSLQPEDFATYYCSQSTHVPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSEGKSSGSGSESKSTEGKSSGSGSESKSTGGSQVQLQESGPGLVKPSETLSLTCTVSGYSITSGYFWNWIRQPPGKGLEWIGYISYDGSNNYNPSLKSRVTISRDTSKNQFSLKLSSVTAADTAVYYCASPSPGTGYAVDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLGK

SEQ ID NO: 14 - protein, 694 aa, synthetic construct
  REGION 1..694: note = I3RB217
EIVLTQSPGTLSLSPGERATLSCRASQSVSSSYLAWYQQKPGQAPRLLIYGASSRATGIPDRFSGSGSGTDFTLTISRLEPEDFAVYYCQQDYGFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSEGKSSGSGSESKSTEGKSSGSGSESKSTGGSEVQLVQSGAEVKKPGESLKISCKGSGYSFTSYWISWVRQMPGKGLEWMGIIDPSDSDTRYSPSFQGQVTISADKSISTAYLQWSSLKASDTAMYYCARGDGSTDLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 699 aa, synthetic construct
  REGION 1..699: note = PSMB365
EIVLTQSPATLSLSPGERATLSCRASQSVSSYLAWYQQKPGQAPRLLIYDASYRATGIPARFSGSGSGTDFTLTISSLEPEDFAVYYCQQRRNWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSEGKSSGSGSESKSTEGKSSGSGSESKSTGGSEVQLLESGGGLVQPGGSLRLSCAASGFTFKSDAMHWVRQAPGKGLEWVSEISGSGGYTNYADSLKSRFTISRDNSKNTLYLQMNSLRAEDTAVYYCARDSYDSSLYVGDYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 120 aa, synthetic construct
  REGION 1..120: note = VH
QVQLQESGPGLVKPSETLSLTCTVSGYSITSGYFWNWIRQPPGKGLEWIGYISYDGSNNYNPSLKSRVTISRDTSKNQFSLKLSSVTAADTAVYYCASPSPGTGYAVDYWGQGTLVTVSS

SEQ ID NO: 17 - protein, 112 aa, synthetic construct
  REGION 1..112: note = VL
DIQMTQSPSSLSASVGDRVTITCRSSQSLVHSNGNTYLHWYQQKPGKAPKFLIYKVSNRFSGVPSRFSGSGSGTDFTLTISSLQPEDFATYYCSQSTHVPFTFGQGTKLEIK

SEQ ID NO: 18 - protein, 447 aa, synthetic construct
  REGION 1..447: note = B17B21 HC
QVQLQESGPGLVKPSETLSLTCTVSGYSITSGYFWNWIRQPPGKGLEWIGYISYDGSNNYNPSLKSRVTISRDTSKNQFSLKLSSVTAADTAVYYCASPSPGTGYAVDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLGK

SEQ ID NO: 19 - protein, 219 aa, synthetic construct
  REGION 1..219: note = B17B21 LC
DIQMTQSPSSLSASVGDRVTITCRSSQSLVHSNGNTYLHWYQQKPGKAPKFLIYKVSNRFSGVPSRFSGSGSGTDFTLTISSLQPEDFATYYCSQSTHVPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 445 aa, synthetic construct
  REGION 1..445: note = I3RB217 HC
EVQLVQSGAEVKKPGESLKISCKGSGYSFTSYWISWVRQMPGKGLEWMGIIDPSDSDTRYSPSFQGQVTISADKSISTAYLQWSSLKASDTAMYYCARGDGSTDLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 215 aa, synthetic construct
  REGION 1..215: note = I3RB217 LC
EIVLTQSPGTLSLSPGERATLSCRASQSVSSSYLAWYQQKPGQAPRLLIYGASSRATGIPDRFSGSGSGTDFTLTISRLEPEDFAVYYCQQDYGFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 10 aa, synthetic construct
  REGION 1..10: note = VH CDR1
GYSFTSYWIS

SEQ ID NO: 23 - protein, 17 aa, synthetic construct
  REGION 1..17: note = VH CDR2
IIDPSDSDTRYSPSFQG

SEQ ID NO: 24 - protein, 9 aa, synthetic construct
  REGION 1..9: note = VH CDR3
GDGSTDLDY

SEQ ID NO: 25 - protein, 12 aa, synthetic construct
  REGION 1..12: note = VL CDR1
RASQSVSSSYLA

SEQ ID NO: 26 - protein, 7 aa, synthetic construct
  REGION 1..7: note = VL CDR2
GASSRAT

SEQ ID NO: 27 - protein, 9 aa, synthetic construct
  REGION 1..9: note = VL CDR3
QQDYGFPWT

SEQ ID NO: 28 - protein, 8 aa, synthetic construct
  REGION 1..8: note = VH CDR1
GYSFTSYW

SEQ ID NO: 29 - protein, 8 aa, synthetic construct
  REGION 1..8: note = VH CDR2
IDPSDSDT

SEQ ID NO: 30 - protein, 11 aa, synthetic construct
  REGION 1..11: note = VH CDR3
ARGDGSTDLDY

SEQ ID NO: 31 - protein, 7 aa, synthetic construct
  REGION 1..7: note = VL CDR1
QSVSSSY

SEQ ID NO: 32 - skipped

SEQ ID NO: 33 - protein, 9 aa, synthetic construct
  REGION 1..9: note = VL CDR3
QQDYGFPWT

SEQ ID NO: 34 - protein, 5 aa, synthetic construct
  REGION 1..5: note = VH CDR1
SYWIS

SEQ ID NO: 35 - protein, 17 aa, synthetic construct
  REGION 1..17: note = VH CDR2
IIDPSDSDTRYSPSFQG

SEQ ID NO: 36 - protein, 9 aa, synthetic construct
  REGION 1..9: note = VH CDR3
GDGSTDLDY

SEQ ID NO: 37 - protein, 11 aa, synthetic construct
  REGION 1..11: note = VL CDR1
RASQSVSSSYL

SEQ ID NO: 38 - protein, 7 aa, synthetic construct
  REGION 1..7: note = VL CDR2
GASSRAT

SEQ ID NO: 39 - protein, 9 aa, synthetic construct
  REGION 1..9: note = VL CDR3
QQDYGFPWT

SEQ ID NO: 40 - protein, 118 aa, synthetic construct
  REGION 1..118: note = I3RB217 VH
EVQLVQSGAEVKKPGESLKISCKGSGYSFTSYWISWVRQMPGKGLEWMGIIDPSDSDTRYSPSFQGQVTISADKSISTAYLQWSSLKASDTAMYYCARGDGSTDLDYWGQGTLVTVSS

SEQ ID NO: 41 - protein, 108 aa, synthetic construct
  REGION 1..108: note = I3RB217 VL
EIVLTQSPGTLSLSPGERATLSCRASQSVSSSYLAWYQQKPGQAPRLLIYGASSRATGIPDRFSGSGSGTDFTLTISRLEPEDFAVYYCQQDYGFPWTFGQGTKVEIK

SEQ ID NO: 42 - protein, 5 aa, synthetic construct
  REGION 1..5: note = VH CDR1
SYWIS

SEQ ID NO: 43 - protein, 17 aa, synthetic construct
  REGION 1..17: note = VH CDR2
IIDPSDSDTRYSPSFQG

SEQ ID NO: 44 - protein, 9 aa, synthetic construct
  REGION 1..9: note = VH CDR3
GDGSTDLDY

SEQ ID NO: 45 - protein, 12 aa, synthetic construct
  REGION 1..12: note = VL CDR1
RASQSVSSSYLA

SEQ ID NO: 46 - protein, 7 aa, synthetic construct
  REGION 1..7: note = VL CDR2
GASSRAT

SEQ ID NO: 47 - protein, 9 aa, synthetic construct
  REGION 1..9: note = VL CDR3
QQDYGFPWT

SEQ ID NO: 48 - protein, 10 aa, synthetic construct
  REGION 1..10: note = VH CDR1
GYSFTSYWIS

SEQ ID NO: 49 - protein, 17 aa, synthetic construct
  REGION 1..17: note = VH CDR2
IIDPSDSDTRYSPSFQG

SEQ ID NO: 50 - protein, 9 aa, synthetic construct
  REGION 1..9: note = VH CDR3
GDGSTDLDY

SEQ ID NO: 51 - protein, 12 aa, synthetic construct
  REGION 1..12: note = VL CDR1
RASQSVSSSYLA

SEQ ID NO: 52 - protein, 7 aa, synthetic construct
  REGION 1..7: note = VL CDR2
GASSRAT

SEQ ID NO: 53 - protein, 9 aa, synthetic construct
  REGION 1..9: note = VL CDR3
QQDYGFPWT

SEQ ID NO: 54 - protein, 10 aa, synthetic construct
  REGION 1..10: note = VH CDR1
GYSFTSYWIS

SEQ ID NO: 55 - protein, 18 aa, synthetic construct
  REGION 1..18: note = VH CDR2
GIIDPSDSDTRYSPSFQG

SEQ ID NO: 56 - protein, 10 aa, synthetic construct
  REGION 1..10: note = VH CDR3
RGDGSTDLDY

SEQ ID NO: 57 - protein, 12 aa, synthetic construct
  REGION 1..12: note = VL CDR1
RASQSVSSSYLA

SEQ ID NO: 58 - protein, 7 aa, synthetic construct
  REGION 1..7: note = VL CDR2
GASSRAT

SEQ ID NO: 59 - protein, 9 aa, synthetic construct
  REGION 1..9: note = VL CDR3
QQDYGFPWT

SEQ ID NO: 60 - protein, 6 aa, synthetic construct
  REGION 1..6: note = VH CDR1
TSYWIS

SEQ ID NO: 61 - protein, 13 aa, synthetic construct
  REGION 1..13: note = VH CDR2
WMGIIDPSDSDTR

SEQ ID NO: 62 - protein, 10 aa, synthetic construct
  REGION 1..10: note = VH CDR3
ARGDGSTDLD

SEQ ID NO: 63 - protein, 8 aa, synthetic construct
  REGION 1..8: note = VL CDR1
SSSYLAWY

SEQ ID NO: 64 - protein, 10 aa, synthetic construct
  REGION 1..10: note = VL CDR2
LLIYGASSRA

SEQ ID NO: 65 - protein, 8 aa, synthetic construct
  REGION 1..8: note = VL CDR3
QQDYGFPW

SEQ ID NO: 66 - protein, 10 aa, synthetic construct
  REGION 1..10: note = VH CDR1
GYSFTSYWIS

SEQ ID NO: 67 - protein, 10 aa, synthetic construct
  REGION 1..10: note = VH CDR2
IIDPSDSDTR

SEQ ID NO: 68 - protein, 9 aa, synthetic construct
  REGION 1..9: note = VH CDR3
GDGSTDLDY

SEQ ID NO: 69 - protein, 12 aa, synthetic construct
  REGION 1..12: note = VL CDR1
RASQSVSSSYLA

SEQ ID NO: 70 - protein, 7 aa, synthetic construct
  REGION 1..7: note = VL CDR2
GASSRAT

SEQ ID NO: 71 - protein, 9 aa, synthetic construct
  REGION 1..9: note = VL CDR3
QQDYGFPWT

SEQ ID NO: 72 - protein, 5 aa, synthetic construct
  REGION 1..5: note = VH CDR1
SDAMH

SEQ ID NO: 73 - protein, 17 aa, synthetic construct
  REGION 1..17: note = VH CDR2
EISGSGGYTNYADSLKS

SEQ ID NO: 74 - protein, 15 aa, synthetic construct
  REGION 1..15: note = VH CDR3
DSYDSSLYVGDYFDY

SEQ ID NO: 75 - protein, 11 aa, synthetic construct
  REGION 1..11: note = VL CDR1
RASQSVSSYLA

SEQ ID NO: 76 - protein, 7 aa, synthetic construct
  REGION 1..7: note = VL CDR2
DASYRAT

SEQ ID NO: 77 - protein, 9 aa, synthetic construct
  REGION 1..9: note = VL CDR3
QQRRNWPLT

SEQ ID NO: 78 - protein, 124 aa, synthetic construct
  REGION 1..124: note = PSMB365 VH
EVQLLESGGGLVQPGGSLRLSCAASGFTFKSDAMHWVRQAPGKGLEWVSEISGSGGYTNYADSLKSRFTISRDNSKNTLYLQMNSLRAEDTAVYYCARDSYDSSLYVGDYFDYWGQGTLVTVSS

SEQ ID NO: 79 - protein, 107 aa, synthetic construct
  REGION 1..107: note = PSMB365 VL
EIVLTQSPATLSLSPGERATLSCRASQSVSSYLAWYQQKPGQAPRLLIYDASYRATGIPARFSGSGSGTDFTLTISSLEPEDFAVYYCQQRRNWPLTFGQGTKVEIK

SEQ ID NO: 80 - protein, 450 aa, synthetic construct
  REGION 1..450: note = PSMB365 HC
EVQLLESGGGLVQPGGSLRLSCAASGFTFKSDAMHWVRQAPGKGLEWVSEISGSGGYTNYADSLKSRFTISRDNSKNTLYLQMNSLRAEDTAVYYCARDSYDSSLYVGDYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCWVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 81 - protein, 214 aa, synthetic construct
  REGION 1..214: note = PSMB365 LC
EIVLTQSPATLSLSPGERATLSCRASQSVSSYLAWYQQKPGQAPRLLIYDASYRATGIPARFSGSGSGTDFTLTISSLEPEDFAVYYCQQRRNWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 698 aa, synthetic construct
  REGION 1..698: note = IAPB63
QSALTQPRSVSGSPGHSVTISCTGTSSDVGDYNYVSWYQQRPGKVPKLLIYDVSKRPSGVPDRFSGSKSGNTASLTISGLQAEDEAIYFCASYAGNYNVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGSEGKSSGSGSESKSTEGKSSGSGSESKSTGGSQVQLVQSGSELKKPGASVKVSCKASGYTFNTYAMNWVRQAPGQGLEWMGWINTNTGNPTYAQGFTGRFVFSLDTSVSTAYLQISSLKAEDTAVYYCARRYFDWLLGAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 83 - protein, 699 aa, synthetic construct
  REGION 1..699: note = B23B49
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSEGKSSGSGSESKSTEGKSSGSGSESKSTGGSQITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK